(19) United States
(12) Patent Application Publication
(10) Pub. No.: US 2026/0321343 A1
(43) Pub. Date: Oct. 15, 2026

(54) DETERMINATION OF B CELL FRACTION IN MIXED SAMPLES

(71) Applicant: Cancer Research Technology Limited, London (GB)

(72) Inventors: Robert Bentham, London, Greater London (GB); Nicholas McGranahan, London, Greater London (GB)

(73) Assignee: Cancer Research Technology Limited, London (GB)

(21) Appl. No.: 19/481,873

(22) PCT Filed: May 10, 2024
(86) PCT No.: PCT/EP2024/062999

§ 371 Date: Nov. 5, 2025

(30) Foreign Application Priority Data
May 12, 2023 (GB) 2307130.1

(51) Int. Cl. G16B 20/10; G16B 30/00

(52) U.S. Cl. CPC G16B 20/10; G16B 30/00

(57) ABSTRACT

Methods for determining the fraction of B lymphocytes of a particular class in a sample comprising genomic material from multiple cell types are described The methods comprise obtaining a read depth profile for the sample comprising read depths derived from sequence data for the sample in a predetermined genomic region, wherein the predetermined genomic region includes at least a region of the IGH locus that undergoes class switch recombination and a region of the IGH locus that does not undergo class switch recombination; obtaining a plurality of read depth ratios (rᵢ) by normalising the read depths in the predetermined genomic region by reference to a baseline read depth derived from a subset of the predetermined genomic region that does not undergo class switch recombination; obtaining one or more summarised read depth ratio values (rB) for respective portions of the region of the IGH locus that are likely to be deleted through class switch recombination; and determining the fraction of B lymphocytes of a particular class in the sample as a function of the one or more summarised read depth ratio values (r_{B}).

### FIELD OF THE DISCLOSURE

The present disclosure relates to methods of estimating the fraction of B lymphocytes in one or more classes, in mixed samples comprising multiple cell types, such as tumour samples or blood samples, based on read depth profiles derived from the samples, and to systems and related products for implementing such methods. Also described are methods of providing a prognosis or a diagnosis for a patient based at least in part on the fraction of B lymphocyte in one or more classes in a sample from the patient.

### BACKGROUND

The immune system is multifaceted, composed of multiple cell types each with both distinct and complementary functions. Immune cells are dynamic, being able to migrate from lymphoid to non-lymphoid tissues to protect the body from pathogens and maintain general health. Therefore, measuring both the quantity and location of immune cell types is vital.

In cancer research, much focus has been on tumour infiltrating leukocytes (TILs). The presence of tumour infiltrating T cells has been linked to the process of immune sculpting and selection pressure for immune evasion mechanisms during tumour evolution, and higher T cell content has shown intrinsic prognostic value and an ability to predict response to immunotherapy such as checkpoint inhibitors. However, the role of other immune cells is not always so clear. For instance, the relationship between tumour infiltrating B cells, outcome and response to treatment have been difficult to ascertain; with B cells in different contexts being shown to either promote or inhibit cancer growth or seemingly just be bystanders with little anti- or pro-tumour activity. Conceivably, the role of infiltrating B cells within a tumour can be delineated by classification into different lineage sub-population. Furthermore, B cells play essential roles in other diseases, such as autoimmune diseases, wherein the amount of B cells of a particular type may be aberrant in number and/or function. However, distinguishing between B cell populations in detail without direct assays of B cell markers with flow cytometry, targeted BCRseq or single cell RNA sequencing is difficult and generally not possible in most genomic data sets that focus on bulk DNA sequencing.

Thus, there exists a need for new methods for determining the abundance of B lymphocytes of a particular class in mixed samples, that alleviate one or more of the drawbacks of existing methods.

### SUMMARY

The present inventors previously developed a method for estimating the total T cell or B cell fraction in a sample (described in WO2023/002046 and Bentham, R. et al., 2021 which are incorporated herein by reference) from whole genome sequencing (WGS) or whole exome sequencing (WES) data, using a signal derived from regions of the TCR or IGH locus that are lost during VDJ recombination to derive a score that directly quantifies the total T or B cell fraction in a mixed sample. In the present work, the inventors postulated that it may be possible to use a similar mathematical framework to quantify different classes of B cells, In particular, the inventors designed a new method to quantify at least the fraction of B cells that are class switched or non-class switched, by quantifying a signal in WGS or WES data that is derived from different regions of the IGH locus than those that are lost during VDJ recombination, in particular from regions that are lost during class switch recombination (CSR). They showed that the proposed method was able to resolve the different classes of B cells, including igM/D, igG, igA, and even B cells of the rare igE class, within various types of samples including both tumour and blood samples. This was extensively validated using a large multi-omic lung cancer patient dataset, and applied to a pan cancer cohort where the inventors demonstrated that metrics derived from the methods of the disclosure were indicative of prognostic.

Accordingly, in a first aspect the present disclosure provides a method for determining the fraction of B lymphocytes of a particular class in a sample comprising genomic material from multiple cell types, the method comprising: obtaining sequence data for the sample, the sequence data comprising a plurality of sequencing reads; obtaining a read depth profile for the sample comprising read depths derived from the sequence data in a predetermined genomic region, wherein the predetermined genomic region includes at least a region of the IGH locus that undergoes class switch recombination and a region of the IGH locus that does not undergo class switch recombination; obtaining a plurality of read depth ratios (rᵢ) by normalising the read depths in the predetermined genomic region by reference to a baseline read depth derived from a subset of the predetermined genomic region that does not undergo class switch recombination; obtaining one or more summarised read depth ratio values (r_{B}) for respective portions of the region of the IGH locus that are likely to be deleted through class switch recombination; and determining the fraction of B lymphocytes of a particular class in the sample as a function of the one or more summarised read depth ratio values (r_{B}); wherein the particular class of B lymphocytes is selected from: igG B cells, igA B cells, igE B cells, igM/D B cells and combinations thereof.

The method may have any one or more of the following optional features.

The method may further comprise providing to a user, for example through a user interface, the determined B lymphocyte fraction and/or any value derived therefrom. A value derived therefrom may comprise a diagnostic or prognostic indication.

As the skilled person understands, the complexity of the operations described herein (due at least to the amount of data that is typically generated by sequencing genomic DNA) are such that they are beyond the reach of a mental activity. Indeed, operations such as aligning reads to a genome reference, determining read depths over significant portions of a genome and computing metrics as described herein from such read depths are clearly beyond the reach of a mental activity. Thus, unless context indicates otherwise (e.g. where sample preparation or acquisition steps are described), all steps of the methods described herein are computer implemented.

The particular class of B lymphocytes may be a class that includes all class switched lymphocytes, i.e. a combination of igG B cells, igA B cells and igE B cells. The region of the IGH locus that undergoes class switch recombination may comprise one or more segments selected from: IGHA2, IGHE, IGHG4, IGHG2, IGHA1, IGHG1, IGHG3, IGHD, and IGHM.

Obtaining a summarised read depth ratio value (r_{B}) for a portion of the region of the IGH locus that is likely to be deleted through class switch recombination may comprise obtaining a summarised read depth ratio value (r_{B}) for a portion of the IGH locus corresponding to a segment selected from: IGHA2, IGHE, IGHG4, IGHG2, IGHA1, IGHG1, IGHG3, IGHD, IGHM, and combinations thereof. For example, obtaining a summarised read depth ratio value (r_{B}) for a portion of the region of the IGH locus that is likely to be deleted through class switch recombination comprises obtaining a summarised read depth ratio value (r_{B}) for a portion of the IGH locus comprising both the IGHD segment and the IGHM segment. Combinations of segments may comprise portions of the region of the IGH locus that is likely to be deleted through class switch recombination that comprises multiple consecutive segments, such as e.g. both the IGHD and the IGHM segment.

A summarised read depth ratio value (r_{B}) for a portion of the IGH locus corresponding to a segment may be the difference or absolute value of the difference between a summarised read depth ratio value for the segment and a summarised read depth ratio value for a neighbouring segment. The neighbouring segment may be a preceding segment. A preceding segment may be a segment that precedes the particular segment in the following order: IGHM, IGHD, IGHG3, IGHG1, IGHA1, IGHG2, IGHG4, IGHE, IGHA2). This may be the reverse order from the order that appears in genomic coordinates. Thus, the neighbouring segment may also be referred to as a subsequent segment, when the order of appearance in genomic coordinates is used.

A baseline read depth may be derived from a subset of the predetermined genomic region that is not expected to be lost in B cells through class switch recombination and/or VDJ recombination. The subsets of the predetermined region may comprise one or both of: a region of the IGH locus that is located before segments that can be deleted through class switching, and a region of the IGH locus that is located after segments that can be deleted through VDJ recombination. A region of the IGH locus that is located before segments that can be deleted through class switching may comprise a region before the IGHA2 segment of the IGH locus, and/or a region of the IGH locus that is located after segments that can be deleted through VDJ recombination comprises a region including the last n V segments of the IGH locus. The subsets of the predetermined region may comprise one or both of: a region with coordinates Hg38: chr14:105566277-105588395 or corresponding coordinates in another human or non-human reference genome; and a region with coordinates Hg38: chr14: 106779812-106879844. The words “last” and “after” in this context may refer to genomic coordinates that are higher than a preceding location (i.e. referring to the conventional order of regions in genomic coordinates). The predetermined genomic region may comprise all or a part of the IGH locus. The predetermined genomic region may comprise a region with genomic coordinates: chr14: 105566277-107288051 in Hg38 or corresponding coordinates in another human or non-human reference genome.

The baseline read depth may be determined from a subset of the IGH locus comprising the hg38 chromosome 14 genomic coordinates: 105566277-105588395 and/or 106779812-106879844, or corresponding coordinates in another human or non-human reference genome (such as e.g. the hg19 chromosome 14 genomic coordinates: 106779812-106879844, and/or 107188051-107288051).

In embodiments where fractions of B cells of a particular class and a total B cell fraction are both determined, the same regions may be used to determine a baseline read depth for calculating the read depth ratios for both the total B cell fraction and the fraction of B cells of a particular class.

The predetermined genomic regions may include one or more exons from the IGH gene locus, and optionally one or more introns from the IGH gene locus. The predetermined genomic region may include a plurality of exons from the IGH gene locus, wherein the plurality of exons comprise at least one exon corresponding to each of the IGHA2, IGHE, IGHG4, IGHG2, IGHA1, IGHG1, IGHG3, IGHD, and IGHM segments. The predetermined genomic region may include least one exon corresponding to each of a V, D and C segment of the IGH gene locus. The plurality of exons may comprise at least one exon corresponding to each of a V, D, J and C segment of the IGH gene locus. The plurality of exons may comprise exons corresponding to multiple V segments of the respective gene locus. The predetermined genomic may include the regions coding for a plurality of V, D, J and/or C segments from the IGH gene locus. The predetermined genomic region may include one or more regions located between V, D, J and/or C segments from the IGH gene locus. The predetermined genomic region may include all exons from the IGH gene locus. The predetermined genomic region may include all of the IGH gene locus (i.e. including both exons and introns).

The predetermined genomic region may include a subset of the exons from the IGH gene locus. The predetermined region may include a subset of the IGH gene locus. For example, the predetermined region may exclude any exon or region that has been determined to be associated with a systematic bias, such as e.g. a bias due to the platform used to collect the read data, a bias associated with the GC content in the exon or region, or a bias associated with the coverage in the exon or region. For example, some exon capture kits have been shown to be associated with biases that lead to coverage in certain exons departing from the expected. Similarly, certain regions may have lower or higher coverage than expected in whole genome sequencing for example due to artefacts in the sequencing or alignment process. Such exons or regions may be identified by comparing read coverages between datasets that are not expected to be subject to the same systematic bias, such as e.g. acquired using different platforms (e.g. different capture kits). For example, the average log R (log read depth normalised to the baseline read depth) for a plurality of candidate exons or regions may be calculated for a first set of samples and a second set of samples (where the two sets of samples are not expected to suffer from the same systematic bias), and the median across each set of samples may be compared for each of the plurality of candidate exons or regions to identify those that differ significantly between the two sets of samples. For example, a difference between median log R above a threshold, such as e.g. 0.5 may be used as a criterion to exclude exons/regions as likely to be subject to bias. Regions with lower or higher coverage than expected may be identified by fitting a model to the mean read depth ratio data across a plurality of samples, such as e.g. a general linear model, and removing any region of a predetermined size (such as e.g. 100 bp, 200 bp, 500 bp, 1000 bp) that is associated with a summarised value (e.g. a mean or median value) that is not within a predetermined distance of the fitted model. Instead or in addition to this, regions with lower or higher coverage than expected may be identified by determining a fraction of lymphocytes for a plurality of samples and performing a genome wide association study to identify single nucleotide polymorphisms that are associated with the determined fraction of lymphocytes, where the presence of an association is indicative of coverage bias in the region of predetermined size comprising the single nucleotide polymorphism. Thus, the method may comprise identifying regions with lower or higher coverage than expected in whole genome sequencing by determining a fraction of lymphocytes for a plurality of samples, performing a genome wide association study to identify single nucleotide polymorphisms that are associated with the determined fraction of lymphocytes, selecting samples including the single nucleotide polymorphism, fitting a model to the mean read depth ratio data across the selected samples, and removing any region of a predetermined size (such as e.g. 100 bp, 200 bp, 500 bp, 1000 bp) that is associated with a summarised value (e.g. a mean or median value) that is not within a predetermined distance of the fitted model.

The method may further comprise obtaining a baseline read depth from a subset of the predetermined genomic region. A baseline read depth may be a summarised read depth value across the subset of the predetermined genomic region that does not undergo class switch recombination. The subset of the region used to obtain the baseline read depth may be a subset of the region that is unlikely to be deleted through VDJ recombination or CSR. The subset of the predetermined genomic region used to obtain the baseline read depth may be a subset of the IGH locus that is unlikely to be deleted through VDJ recombination or class switch recombination. Such as subset may comprise a plurality of regions. For example, the subset may comprise a first region of the IGH locus that is located after segments that can be deleted through VDJ recombination, comprising a region including the last n V segments of the IGH locus, where n can be e.g. 5, 6, 7, 8, 9, 10, 11, 12, 13, 14, 15, specifically 13. For example, the first region may comprise the final ~10k bases of the IGH locus. The subset may comprise a second region, including all or a part of the region from the start of the IGH locus to the start of the first class switching segment IGHA2. The subset of the region of interest that is unlikely to be deleted through VDJ recombination or CSR may comprise the n first V segments of the genomic locus that undergoes VDJ recombination. The subset of the region of interest that is unlikely to be deleted through VDJ recombination or CSR recombination may comprise a region before the first segment that undergoes CSR. Preferably, the subset of the region of interest used to obtain the baseline read depth does not comprise any D or J segment of the genomic locus that undergoes VDJ recombination. Indeed, it is possible according to the methods disclosed herein to use the beginning, the end, or both the beginning and the end of the IGH locus under investigation (instead of both), or other regions close to the IGH locus that can be assumed to have copy number values unaffected by VDJ recombination or CSR. The words “start”, “beginning”, “last” and “end” in this context may refer to the conventional order of regions in genomic coordinates (i.e. the start of a locus has a lower coordinate than the end of a locus).

Without wishing to be bound by theory, it is believed that the use of longer normalisation regions (e.g. using both the beginning and end regions, and/or increasing lengths of the beginning region) advantageously compensates for noise that is typical in read depth data. For example, the beginning region can be extended to include a number of V segments that are unlikely to be frequently lost, or even regions outside of the gene if this data is available (such as e.g. when using whole genome sequencing data). Further, it is believed that the use of the signal at the beginning of the gene is beneficial because the signal associated with VDJ recombination is closer to the end of the gene than to its beginning (due to the J segments being clustered close to the end of the gene, whereas the V segments are more spread out). Additionally, it would also be possible to use a region outside of the VDJ and CSR loci under investigation, such as e.g. before or after the locus. However, increasing distances from the locus of interest increase the likelihood that the signal seen will correspond to a different copy number event in the cancer cells, and hence no longer reflect the tumour copy number at the locus under investigation. The parameter n may be determined using appropriate training data, for example by choosing the value of n that results in the most accurate estimate of lymphocyte fraction across the training data, when compared against an orthogonal metric of lymphocyte fraction. An orthogonal metric of lymphocyte fraction may be obtained for examples based on transcriptomic data and/or histopathology data. The parameter n may be between 8 and 16, between 10 and 14, such as e.g. 10, 11 or 12.

Obtaining a read depth profile for the sample may comprise obtaining read depths along one or more genes segments from the constant region of the IGH locus. The one or more segments may have genomic coordinates selected from:
a) IGHA2, hg38 co-ordinates: chr14:105583731-105588395, hg19 coordinates: chr14:106053226-106054732, or corresponding coordinates in another human or non-human reference genome;
b) IGHE, hg 38 coordinates: chr14:105597691-105601728, hg19 coordinates: chr14:106064224-106068065, or corresponding coordinates in another human or non-human reference genome;
c) IGHG4, hg38 coordinates: chr14:105620506-105626066, hg19 coordinates: chr14:106090687-106092403, or corresponding coordinates in another human or non-human reference genome;
d) IGHG2, hg38 coordinates: chr14:105639559-105644790, hg19 coordinates: chr14:106109389-106111127, or corresponding coordinates in another human or non-human reference genome;
e) IGHA1, hg38 coordinates: chr14:105703995-105708665, hg19 coordinates: chr14:106173457-106175002, or corresponding coordinates in another human or non-human reference genome;
f) IGHG1, hg38 coordinates: chr14:105736343-105743071, hg19 coordinates: chr14:106202680-106209408, or corresponding coordinates in another human or non-human reference genome;
g) IGHG3, hg38 coordinates: chr14:105764503-105771405, hg19 coordinates: chr14:106235439-106237742, or corresponding coordinates in another human or non-human reference genome;
h) IGHD, hg38 coordinates: chr14:105836765-105845677, hg19 coordinates: chr14:106303099-106312010, or corresponding coordinates in another human or non-human reference genome; and
i) IGHM, hg38 coordinates: chr14:105851705-105856218, hg19 coordinates: chr14:106320349-106322323, or corresponding coordinates in another human or non-human reference genome.

The predetermined genomic region may further include a region of the IGH locus that undergoes VDJ recombination. The method may further comprise determining the total B lymphocyte fraction by: obtaining a further summarised read depth ratio value (r_{B}) for a subset of the region of the IGH locus that is likely to be deleted through VDJ recombination; and determining the total fraction of B lymphocytes (f^{tot}) in the sample as a function of the further summarised read depth ratio value (r_{B}). The subset of the region of the IGH locus that is likely to be deleted through VDJ recombination may comprises a region located between the end of the last J segment of the genomic locus that undergoes VDJ recombination, and the start of the first V segment of the genomic locus that undergoes VDJ recombination. The subset of the IGH locus that is likely to be deleted through VDJ recombination may comprise one or more D segments (such as e.g. all D segments) of the genomic locus that undergoes VDJ recombination. The subset of the region of the IGH locus that is likely to be deleted through V(D)J recombination may be Hg38: chr14: 105865679-105939755 or corresponding coordinates in another human or non-human reference genome.

The subset of the region of interest that is likely to be deleted through VDJ recombination may comprise a region located between the end of the last J segment of the genomic locus that undergoes VDJ recombination, and the start of the first V segment of the genomic locus that undergoes VDJ recombination. The words “start”, “beginning”, “last” and “end” in this context may refer to the conventional order of regions in genomic coordinates (i.e. the start of a locus has a lower coordinate than the end of a locus). The subset of the region of interest that is likely to be deleted through VDJ recombination may comprise one or more D segments (such as e.g. all D segments) of the genomic locus that undergoes VDJ recombination. The subset of the region of interest that is likely to be deleted through VDJ recombination preferably does not include any V or C segment. Instead or in addition to this, the subset of the region of interest that is likely to be deleted through VDJ recombination preferably does not include any J segment. The subset of the region of interest that is likely to be deleted through VDJ recombination may comprise, consist of, or be comprised in a gap between the final V gene segment and a first segment that encodes a J segment of the genomic locus that undergoes VDJ recombination. The subset of the region of interest that is likely to be deleted through VDJ recombination may be a region of the IGH locus where VDJ recombination is maximum (i.e. where the summarised read depth ratio value is minimum), also referred to as a “region of maximum VDJ recombination”). The subset of the region of interest that is likely to be deleted through VDJ recombination may be defined as the gap between the final IGH J segment and the first IGH V segment, i.e. region chr14:105865679-105939755 in Hg38. The subset of the region of interest that is likely to be deleted through VDJ recombination may be defined as the gap between J segment IGHJ1P and V segment IGHV6-1. Regions of maximum VDJ recombination may be defined as a region located at least partially around or within the region between the final V gene segment and the first J gene segment. The subset of the region of interest that is likely to be deleted through VDJ recombination may comprise a region corresponding to any V or J segment of the genomic locus that undergoes VDJ recombination. This may be used when the fraction of lymphocytes is a fraction of B cells including any gene segment in IGH gene locus, or a fraction of B cells including any gene segment listed in Table 1 or corresponding gene segments in another species.

The portions of the region of the IGH locus that are likely to be deleted through class switch recombination may be selected from portions of the IGH locus corresponding to a segment selected from: IGHA2, IGHE, IGHG4, IGHG2, IGHA1, IGHG1, IGHG3, IGHD, and IGHM; and determining the fraction of B lymphocytes of a particular class in the sample as a function of the one or more summarised read depth ratio values (r_{B}) may comprise determining, for each of one or more of said segments: the fraction of B lymphocytes (f^{class}) that include the segment as a function of the summarised read depth ratio value (r_{B}) associated with the segment. The summarised read depth ratio value associated with a particular segment may be the difference or absolute value of a difference between a read depth ratio value associated with the segment and a read depth ratio value associated with the preceding segment. A preceding segment may be a segment that precedes the particular segment in the following order: IGHM, IGHD, IGHG3, IGHG1, IGHA1, IGHG2, IGHG4, IGHE, IGHA2). This may be the reverse order from the order that appears in genomic coordinates. The summarised read depth ratio value (r_{B}) associated with a segment may be indicative of the fraction of cells that include a deletion breakpoint before the segment. The particular class of B lymphocytes may be or may comprise the igG B cell class, the one or more summarised read depth ratio values (r_{B}) may comprise summarised read depth ratio values (r_{B}) for respective portions of the region of the IGH locus each comprising a segment selected from IGHG1, IGHG2, IGHG3, and IGHG4, and the fraction of B lymphocytes in the igG B class may be calculated as the sum of: the fraction of B lymphocytes that include a deletion breakpoint before the IGHG1 segment, the fraction of B lymphocytes that include a deletion breakpoint before the IGHG2 segment, the fraction of B lymphocytes that include a deletion breakpoint before the IGHG3 segment, and the fraction of B lymphocytes that include a deletion breakpoint before the IGHG4 segment. The particular class of B lymphocytes may be or may comprise the igA B cell class, the one or more summarised read depth ratio values (r_{B}) may comprise summarised read depth ratio values (r_{B}) for respective portions of the region of the IGH locus each comprising a segment selected from IGHA1 and IGHA2, and the fraction of B lymphocytes in the igA B class may be calculated as the sum of: the fraction of B lymphocytes that include a deletion breakpoint before the IGHA1 segment and the fraction of B lymphocytes that include a deletion breakpoint before the IGHA2 segment. The particular class of B lymphocytes may be or may comprise the igE B cell class, the one or more summarised read depth ratio values (r_{B}) may comprise a summarised read depth ratio values (r_{B}) for a portion of the region of the IGH locus that comprises the IGHE segment, and the fraction of B lymphocytes in the IgE class may be equal to the fraction of B lymphocytes that include a deletion breakpoint before the IGHE segment. The particular class of B lymphocytes may be or may comprise the igM/D class, the one or more summarised read depth ratio values (r_{B}) may comprise summarised read depth ratio values (r_{B}) for respective portions of the region of the IGH locus each comprising a segment selected from IGHG1, IGHG2, IGHG3, IGHG4, IGHA1, IGHA2 and IGHE, the method may comprise determining the total B lymphocyte fraction by obtaining a further summarised read depth ratio value (r_{B}) for a subset of the region of the IGH locus that is likely to be deleted through VDJ recombination, and determining the total fraction of B lymphocytes (f^{tot}) in the sample as a function of the further summarised read depth ratio value (r_{VDJ}), and the fraction of B lymphocytes in the igM/D class may be calculated as the total B lymphocyte fraction minus the sum of: the fraction of B lymphocytes that include a deletion breakpoint before the IGHG1 segment, the fraction of B lymphocytes that include a deletion breakpoint before the IGHG2 segment, the fraction of B lymphocytes that include a deletion breakpoint before the IGHG3 segment, the fraction of B lymphocytes that include a deletion breakpoint before the IGHG4 segment, the fraction of B lymphocytes that include a deletion breakpoint before the IGHA1 segment, the fraction of B lymphocytes that include a deletion breakpoint before the IGHA2 segment, and the fraction of B lymphocytes that include a deletion breakpoint before the IGHE segment.

The method may comprise determining the fraction of B lymphocytes of a plurality of classes, wherein the classes comprise: igG B cells, igA B cells, igE B cells and igM/D B cells. The method may comprise determining the fraction of class switched B lymphocytes as the sum of the igG, igA, and igE B lymphocyte fractions. The igM/D B cells may also be referred to as “non-class switched B lymphocytes”. The method may comprise determining the fraction of class switched B lymphocytes as the sum of the igG, igA, and igE B lymphocyte fractions. Instead or in addition to this, the method may comprise determining the fraction of class switched B lymphocytes as a function of a summarised read depth ratio value (r_{B}) in a region of maximum class switch recombination. The region of maximum class switch recombination may comprise at least part of (such as e.g. all of) segments IGHM and/or IGHD.

Determining the fraction of B lymphocytes of a particular class in the sample as a function of the one or more summarised read depth ratio values (r_{B}) may comprise determining, for each of one or more segments of the IGH gene for which a summarised read depth ratio values (r_{B}) has been obtained, the fraction of B lymphocytes (f^{class}) that include a deletion breakpoint before the segment as a function of the summarised read depth ratio value (r_{B}) associated with the segment. The method may comprise determining the total fraction of B lymphocytes by obtaining a further summarised read depth ratio value (r_{B}) for a subset of the region of the IGH locus that is likely to be deleted through VDJ recombination and determining the total fraction of B lymphocytes (f^{tot}) in the sample as a function of the further summarised read depth ratio value (r_{B}).

The fraction of B lymphocytes (f^{class}) that include a deletion breakpoint before the segment and/or the total fraction of B lymphocytes (f^{tot}) and/or the total fraction of class switched B lymphocytes (f^{class}) may be determined as a function of the respective summarised read depth ratio value, the fraction of abnormal cells (p) in the mixed sample, where abnormal cells are those that are aneuploid in the region of the IGH locus, and the copy number of the abnormal cells in the region of the IGH locus (ψ^{T}).

Determining the fraction of B lymphocytes that include a deletion breakpoint before the segment (f=f^{class}) in the sample and/or determining the total fraction of B lymphocytes (f=f^{tot}) and/or the total fraction of class switched B lymphocytes (f^{class}) may comprise determining the value of:

$$f = ( 1 - p + \frac{p}{2} {Ψ}_{T} ) - ( 1 - p + \frac{p}{2} {Ψ}_{T} ) {2}^{\frac{{τ}_{B}}{γ}} \qquad ( 7 )$$

where γ is a constant; or

$$f = 1 - {2}^{\frac{{r}_{B}}{γ}} \qquad ( 3 )$$

where γ is a constant.

All read depth ratio values may be expressed in log scale, e.g. as log base 2 read depth ratios. Thus, a read depth ratio value of 0 may refer to the read depth being the same as the baseline read depth.

The fraction of abnormal cells (p) in the mixed sample, and the copy number of the abnormal cells in the predetermined region of interest (ψ_{T}) may be determined by obtaining an abnormal cell-specific copy number profile using methods known in the art, such as e.g. the ASCAT method described in Van Loo et al. 2010.

The parameter γ may be adjusted dependent on the analytic platform used to obtain the read depth data. For example, when using Illumina Hiseq, the value of γ may be =1 (i.e. the parameter can be removed from all equations). Without wishing to be bound by theory, it is believed that the parameter γ captures platform-related “compaction” of log R profiles (also referred to as ‘r’, log R is the log ratio of read depths in a region of interest vs a reference region, in the present case) compared with theoretically expected values. For example, r should be equal to zero when comparing regions expected to have the same copy numbers. Thus, this parameter may be set for a particular platform by comparison with expected values in control settings. As the skilled person understands, equation (3) is equivalent to equation (7) if ψ_{T}=2 and/or p=0 (i.e. the sample does not contain abnormal cells, or any cells in the sample that may be considered abnormal by reference to other regions are not aneuploid in the region of interest). Thus, equation (3) may be used when the sample is not expected to contain abnormal cells (e.g. a germline sample). Equation (3) may also be used when p and/or ψ_{T} are unknown, uncertain or unreliable (i.e. when the proportion of abnormal cells and/or their average copy number in the predetermine region of interest is unknown, uncertain or unreliable). For example, equation (3) may be used if the result of equation (7) exceeds 1−p. Without wishing to be bound by theory, the inventors believe that equation (3) will be suitable even if in fact the sample does contain abnormal cells, as long as the lymphocytes represent a relatively small proportion of the cells in the mixed sample.

The method may further comprise fitting a model to the plurality of read depth ratios (rᵢ), and wherein obtaining one or more summarised read depth ratio values for respective portions of the region of the IGH locus that are likely to be deleted through class switch recombination comprises obtaining the one or more summarised read depth ratio values based on the value of the model in the respective portion of the region of the IGH locus that is likely to be deleted through class switch recombination, optionally wherein the model is a general linear model, a piecewise constant linear model with a plurality of breakpoints selected to correspond to locations of a plurality of segments in the constant region of the IGH locus, or a Bayesian model modelling segment usage from the read depth ratios and a prior distribution of usage of each of a plurality of segments in the constant region of the IGH locus. Fitting a model to the plurality of read depth ratios (rᵢ) may comprise fitting a plurality of models to respective portions of the IGH locus. For example, a first model may be fitted to a first region of the IGH locus that undergoes VDJ recombination, and a second model may be fitted to a second region of the IGH locus that undergoes class switch recombination. The model may be a piecewise constant linear model fitted to the plurality of read depth ratios using one or more of the following constraints: breakpoints between constant sections are restricted to locations of the ends of a plurality of segments in the constant region of the IGH locus, the read depth ratio value at the end of the IGHM segment in the IGH locus must be 0, and the read depth ratio after the IGHG3 segment in the IGH locus must be less or equal to the read depth ratio for a subset of the region of the IGH locus that is likely to be deleted through VDJ recombination. For example, the model may be fitted with the constraint that the read depth ratio value for a segment between the end of the IGHM segment and the first J segment (e.g. IGHJ6) in the IGH locus must be 0. Such a segment may have genomic coordinates chr14: 105856218-105863258 (Hg38). For example, the model may be fitted with the constraint that the value of the model at the IGHD and/or IGHM segments (or a single segment comprising both IGHD and IGHM) must be equal or less than the value of the model determined for the subset of the region of the IGH locus that is likely to be deleted through VDJ recombination.

The method may further comprise fitting a model to the plurality of read depth ratios (rᵢ), and wherein obtaining a further summarised read depth ratio (r_{B}) for a subset of the region of the IGH locus that is likely to be deleted through VDJ recombination comprises obtaining the further summarised read depth ratio value based on the value of the model in the subset of the region of the IGH locus that is likely to be deleted through VDJ recombination. The model may be a general linear model, a piecewise constant linear model with a plurality of breakpoints selected to correspond to locations of a plurality of V and J genes in the IGH locus, or a Bayesian model modelling segment usage from the read depth and a prior distribution of usage of each of a plurality of V and J genes in the IGH locus. The model may be a piecewise constant linear model fitted to the plurality of read depth ratios using one or more of the following constraints: breakpoints between constant sections are restricted to locations of the ends of a plurality of V and J segments in the IGH locus, the read depth ratio value before the first V segment must be 0, the read depth ratio value must decrease monotonically between the first V segment and the last V segment, the read depth ratio value must increase monotonically between the first J segment and the last J segment, and the read depth ratio value after the last J segment must be 0. A model fitted to the plurality of read depth ratios may capture the log read depth ratio as a function of the genome position along the predetermined genomic region. The plurality of read depth ratios may also be referred to herein as a “read depth ratio profile”. As the skilled person understands, the plurality of read depth ratios are organised relative to each other along genomic coordinates and as such reference to fitting a model to this plurality of value refers to fitting model to the data series comprising the plurality of read depth ratios as a function of genomic coordinates.

In general, a summarised value may be a median, average, trimmed average or any other known statistical metric of centrality of a plurality of values. A summarised read depth ratio value for a region may be a statistical metric of centrality for the plurality of read depth ratio values within the region. In embodiments where a model (such as e.g. a generalised additive model) is fitted to the read depth ratio profile, a summarised read depth ratio value for a region may be obtained as a statistical metric of centrality for the value of the fitted model within the region. For example, the average of the fitted model across the region may be used. In embodiments where a piecewise constant model is fitted to the read depth ratio profile, a summarised read depth ratio value for a region may be obtained as the value of the fitted model for the segment corresponding to the region or the plurality of segments corresponding to the region, or the value of the model for a segment within the region. For example, using a piecewise constant model constrained as described above, a summarised read depth ratio value for a subset of the region of interest that is likely to be deleted through VDJ recombination may be chosen as the minimum value of the model (which is also equal to the maximum deviation from 0 of the model). Further, using a piecewise constant model constrained as described above, a summarised read depth ratio value for a subset of the region of interest that is likely to be deleted through class switch recombination may be chosen as the difference between the value of the model in that subset (segment) and the value of the model in the preceding segment. In other words, the summarised read depth ratio value for usage of a particular segment may be a difference or absolute value of a difference between a summarised read depth ratio value for the gene segment and a summarised read depth ratio value for the preceding gene segment. The summarised read depth ratio values may be summarised log read depth ratios. The summarised read depth ratios may be obtained from the values of a model fitted to the read depth ratios. Any logs may be base 2 logarithms.

The method may further comprise obtaining a likelihood for the model fitted to the plurality of read depth ratios, wherein a likelihood below a predetermined threshold is indicative of excessive noise in the sample. The method may further comprise obtaining a plurality of candidate models and one or more statistical metrics (such as a likelihood and/or confidence interval) associated with the fitting of the plurality of candidate models, and selecting a candidate model based on the statistical metric(s). For example, a model with highest likelihood with a confidence interval within a predetermined range may be selected. Obtaining a plurality of candidate models may comprise obtaining a predetermined number of models, such as e.g. 10, 30, 50 or 100 models.

A summarised read depth ratio value (r_{B}) for a portion of the region of the IGH locus that is likely to be deleted through class switch recombination may be obtained as the difference or absolute value of a difference between a read depth ratio value associated with the portion of the region of the IGH locus and a read depth ratio value associated with the preceding portion of the region of the IGH locus. The read depth ratio value associated with the portion of the region of the IGH locus may be a summarised value. The summarised value may be derived from a model. A summarised value associated with any particular region (including a subset of the region of the IGH locus that is likely to be deleted through VDJ recombination, or a portion of the region of the IGH locus that is likely to be deleted through class switch recombination) may be a single value that is derived from a plurality of read depth ratios in the particular region. The read depth ratios may be read depth ratios obtained by fitting a model to the plurality of read depth ratios in the predetermined genomic region.

A summarised read depth ratio value (r_{B}) for a subset of the region of the IGH locus that is likely to be deleted through VDJ recombination may be a summarised value associated with the subset of the region of the IGH locus that is likely to be deleted through VDJ recombination.

A summarised read depth ratio value (r_{B}) for a portion of the region of the IGH locus that is likely to be deleted through class switch recombination may be obtained as the difference or absolute value of a difference between a read depth ratio value associated with the portion of the region of the IGH locus and a read depth ratio value associated with the preceding portion of the region of the IGH locus. Alternatively, a summarised read depth ratio value (r_{B}) for a portion of the region of the IGH locus that is likely to be deleted through class switch recombination may be a summarised value associated with a subset of the region of the IGH locus that is likely to be deleted through CSR in all class switched B lymphocytes. This may also be referred to as a region of maximum CSR. This region may include at least part of one or both of the IGHM and IGHD segments. This region may be a region location between the IGHG3 segment and the first J segment in the IGH locus. For example, a region of maximum CSR may be located at chr14:105771405-105863198 in Hg38 or corresponding coordinates in another human or non-human reference genome.

Obtaining a read depth profile may comprise obtaining a plurality of raw read depth values across the predetermined genomic region and smoothing the read depth values. Smoothing the read depth values may comprise replacing the raw read depth values by a corresponding rolling median over a window of a predetermined width. The predetermined width of the window may be selected between approximately 20 bp and approximately 200 bp, or between approximately 50 bp and approximately 150 bp. For example, the read depth profile may comprise values obtained by calculating a rolling median in 50 bp windows along the predetermined region of interest. Suitable sizes of windows can be determined empirically for a particular data set, type of data or context, by comparing the lymphocyte fraction estimates obtained using the present method with various candidate window sizes to orthogonal metrics of lymphocyte fractions.

Obtaining a read depth profile for the sample may comprise obtaining raw read depth values from the sequence data in the predetermined genomic region and correcting said raw read depth values for GC content. Obtaining a read depth profile for the sample may comprise obtaining raw read depth values from the sequence data in the predetermined genomic region and correcting said raw read depth values (or said read depth values corrected for GC content) for copy number alterations in the predetermined region. Correcting raw or GC corrected read depths values for copy number alterations in the predetermined regions may comprise correcting for germline copy number alterations (e.g. in the case of a germline sample). Correcting raw or GC corrected read depths values for copy number alterations in the predetermined regions may comprise correcting for germline copy number alterations and somatic copy number alterations (e.g. for some tumour samples).

The sequence data may be from whole genome sequencing. The sequence data may be from whole genome sequencing with a depth of at least 2×, preferably at least 5×. The sequence data may be from whole exome sequencing, whole genome sequencing, or panel sequencing data. The read depth profile may be obtained from whole exome sequencing data that has a sequencing depth of at least 10×, preferably at least 15×, preferably at least 20×, more preferably at least 30×. The read depth profile may be obtained from whole genome sequencing data that has a sequencing depth of at least at least 2×, at least 5×, or at least 10×. The read depth profile may be obtained from sequencing data that has been filtered to remove exons that have a sequencing depth below a threshold, such as e.g. 10×, preferably 15×. The read depth profile may be obtained from sequencing data that has been filtered to remove regions (e.g. defined using a window of fixed size such as e.g. 1000 bp) that have a sequencing depth below a threshold, such as e.g. 2×. Sequencing data comprising fewer than a predetermined number of exons in the predetermined genomic region of interest with a sequencing depth above a threshold (such as e.g. 10× or 15×) may be discarded. For example, data comprising fewer than 30 exons in the predetermined region of interest (e.g. the IGH locus) with a sequencing depth above a threshold (e.g. 2×) may be discarded. In such cases, new sequence data may be acquired for the sample. The read depth profile may be calculated at single base resolution or for each of a plurality of bins of a predetermined size, such as e.g. 50 bp, 100 bp, or more.

The sample may be a sample from a subject who has or is suspected of having an autoimmune disease. The sample may be a blood sample or a tissue sample. The sample may be a sample from a subject who has been diagnosed as having cancer. The sample may be a blood sample or a tumour sample. The method may further comprise obtaining a sample comprising genomic material from multiple cell types, from a subject, and/or obtaining sequence data from a sample comprising genomic material from multiple cell types, from a subject through one or more in vitro steps.

The method may further comprise repeating the method of any embodiment of the present aspect for one or more further sample(s), wherein the first and further sample(s) have been obtained from the same subject and wherein the first and further samples are tumour samples. The method may further comprise obtaining one or more summarised fraction(s) of B lymphocytes of one or more particular classes based on the fraction of B lymphocytes in these classes for the first and further samples. A summarised fraction of lymphocytes may be obtained the mean fraction across the first and further samples, the minimum fraction across the first and further samples, the maximum fraction across the first and further samples, and/or the fold difference between the minimum fraction across the first and further samples and the maximum fraction across the first and further samples. Where a plurality of samples are available for a subject, one or more of the summarised fraction(s) of B lymphocytes of a particular class may be used instead of the individual lymphocyte fraction for single samples to provide a prognosis as will be described further below. For example, the minimum fraction across the plurality of samples, the minimum and maximum fraction across the plurality of mixed samples, and/or the fold difference between the minimum fraction across the first and further mixed samples and the maximum fraction across the first and further mixed samples may be used to provide a prognosis for the subject. Preferably, the prognosis is provided using the minimum fraction across the plurality of samples.

The method may further comprise providing to a user, for example through a user interface, the determined one or more B lymphocyte fractions and/or any value derived therefrom.

The methods of the present disclosure find use in multiple clinical contexts where different diagnosis and/or prognosis may be associated with different levels of B lymphocytes of one or more particular classes in samples from subjects.

Thus, according to a second aspect, there is provided a method of providing a prognosis for a subject that has been diagnosed as having cancer, the method comprising determining the fraction of B lymphocytes of a particular class in one or more tumour or blood samples from the subject using the method of any embodiment of the first aspect. The method may comprise determining the fraction of igM/D B lymphocytes in a blood sample from the subject, and determining whether the subject belongs to a first group associated with a first range of values of the fraction of igM/D B lymphocytes or a second group associated with a second, lower range of values of the fraction of igM/D B lymphocytes, wherein the first group is associated with a better prognosis than the second group.

According to a third aspect, there is provided a method of diagnosing a subject as having an auto-immune disease characterised by an increase in the fractions of B lymphocytes of one or more particular classes, the method comprising determining the fraction of B lymphocytes of the one or more particular classes in a sample from the subject using the method of any embodiment of the first aspect.

Also described is a method of providing a prognosis for a subject that has been diagnosed as having cancer, the method comprising determining a fraction of B lymphocytes in one or more predetermined classes in one or more tumour samples from the subject using the method of any embodiment of the first aspect. Also described is a method of monitoring a subject that has been diagnosed as having cancer, the method comprising determining a fraction of B lymphocytes in one or more predetermined classes in one or more tumour samples from the subject acquired at a first time point and in one or more tumour samples from the subject acquired at a further time point, using the method of any embodiment of the first aspect. For example, the first time point may be before treatment and the further time point may be after treatment. Alternatively, the first and further time points may both be after treatment.

Also described is a method of determining whether a subject has an autoimmune disease, the method comprising determining a fraction of B lymphocytes in one or more predetermined classes in one or more samples from the subject using the method of any embodiment of the first aspect.

According to a further aspect, the present disclosure provides a system comprising: at least one processor; and at least one non-transitory computer readable medium containing instructions that, when executed by the at least one processor, cause the at least one processor to perform the method of any one or more embodiments of any one or more of the preceding aspects, or any one or more steps of any method described herein.

The system may further comprise, in operable connection with the processor, one or more of: a user interface, wherein the instructions further cause the processor to provide, to the user interface for outputting to a user, at least the estimated value of a B lymphocyte fraction or a value derived therefrom; one or more sequence read depth data acquisition device (such as e.g. a sequencing device); one or more data stores, such as e.g. a sequence read depth data store.

According to a further aspect, there is provided a non-transitory computer readable medium or media comprising instructions that, when executed by at least one processor, cause the at least one processor to perform the method of any one or more embodiments of any one or more of the preceding aspects, or any one or more steps of any method described herein.

According to a further aspect, there is provided a computer program comprising code which, when the code is executed on a computer, causes the computer to perform the method of any one or more embodiments of any one or more of the preceding aspects, or any one or more steps of any method described herein.

The present invention includes the combination of the aspects and preferred features described except where such a combination is clearly impermissible or is stated to be expressly avoided. These and further aspects and embodiments of the invention are described in further detail below and with reference to the accompanying examples and figures.

### BRIEF DESCRIPTION OF THE FIGURES

FIG. 1 is a flowchart illustrating schematically a method of determining a B lymphocyte fraction in a mixed sample.

FIG. 2 shows an embodiment of a system for determining a B lymphocyte fraction in a mixed sample.

FIG. 3 illustrates schematically the biological concept behind the determination of the lymphocyte fraction in a mixed sample as described herein, using the VDJ recombination signal.

FIG. 4 shows schematically how the signal illustrated in FIG. 3 can be converted into a lymphocyte (here illustrated as T cell) fraction estimate.

FIG. 5 shows A. Theoretical values for naïve IGH B cell fraction scores for a range of local copy number and tumour purity values. Points and lines are coloured by the actual B cell fraction with the straight horizontal lines indicating where the points should be. B. Distribution of local copy number of the TCRA gene across the TRACERx100 cohort. C. Distribution of tumour purity across TRACERx100 cohort. D. Exact vs naïve score for B cell fraction by copy number for the TRACERx100 cohort, as can be seen in cases where copy number is 1 the naïve score is an overestimate and in other cases where it is above 2 it is an underestimate.

FIG. 6 illustrates an implementation of the method of FIG. 4 for WGS data. A. A read depth is obtained along the IGH locus. B. A read depth ratio is calculated which is the ratio of the read depth at a location divided by a summarised read depth (illustrated as median read depth) over normalising regions (here illustrated as regions at the start and end of the locus). C. A model is fitted to the read depth ratio. Two alternatives are shown: a generalised additive model (GAM model) that uses a bin-based approach for GC correction and quality control, and a segment model that uses the known location of V and J segments. The segment model has the following constraints: 1. Model log read depth ratio as a linear model consisting of constant segments aligning to positions of V(D)J segments. 2. Model has value of 0 before first V segment. 3. V segments are monotonically decreasing in the order they appear in the genome e.g. TRVA2<TRVA1. 4. J segments are monotonically increasing in the order they appear in the genome. 5. Model has value of 0 after last segment.

FIG. 7 shows an overview of a tool incorporating methods described herein and example of use thereof. A. Diagrammatic overview of a tool for determining the T cell and B cell fraction from WGS data. B. Diagram showing possible class switching deletion events following VDJ recombination at the IGH locus resulting in B cells with different output antibodies. C. Example output from methods described herein, showing the IGH locus for TRACERx sample CRUK0004 R2, showing predicted sample B cell fraction of 0.25 and IGHV segment usage (right panel) as well class switching percentages. D. Example output from methods described herein including correction for copy number alterations, showing the IGH locus for TRACERx sample CRUK0004 region 2, showing predicted sample B cell fraction of 0.25 and IGHV segment usage (right panel) as well class switching percentages.

FIG. 8 shows the results of an analysis of downsampled WGS data using the methods described herein. A. Scatter plot of downsampled IGH B cell fraction at 5× average coverage compared to non-downsampled values. B. Top panel: Track of B cell fraction of TRACERx100 WGS samples nested downsampled at different depths. Bottom panel: correlation of B cell fraction at 60× with samples downsampled to 50, 40, 30, 20, 10, 5, 2, 1, 0.5, 0.1×.

FIG. 9 shows the results of validation of the methods described herein by comparison with orthogonal information derived from RNA sequencing. A. Scatter plots showing IGH B cell fractions of the different class switched compartments as calculated from TRACERx WGS data versus the B cell Danaher score from matched RNAseq samples. B. Scatter plot of IGH B cell fraction versus sample matched Danaher score for B cells from the TRACERx100 cohort, divided by the different possible class switched segment. C. Heatmap of Spearman correlation of B cell class switching fraction predictions to RNA seq gene expression signatures for different cell types from the Danaher, Davoli, TIMER, Cibersort, xCell and EPIC methods in the TRACERx100 data set. D-F. Correlation of proportion of RNA reads mapping to IGH from the TRUST algorithm with different class switched proportions to the proportion of class switched B cells as measured from DNA with the methods described herein in TRACERx100. G-I. Correlation of the number of unique IGHV clones as measured by MiXCR from RNAseq data to sample matched B cell fraction as measured by WGS for either igM/D, igG or igA B cells.

FIG. 10 shows the results of investigation of gene expression analysis of samples with different B cell class proportions identified using the methods described herein A. Output from LimmaVoom dividing the TRACERx RNAseq samples into high and low groups based on the median value of predicted IGHG1, IGHA1 of non-class switched igM/D B cell fractions, red points show those genes within the Travagilini lung B cell gene signature, with P values derived from a GSEA analysis of all cell type signature gene sets defined by MSigDB. B. Results from LimmaVoom dividing TRACERx RNAseq samples by high and low igM/D fraction, highlighting the significant cell type signature gene set for fetal lung ciliated epithelial cells. C. Comparison of the t values from the limmaVoom analysis for IGHG1 vs IGHA1 (top left panel), IGHG1 vs igM/D (top left and bottom panels) with only those genes within the Travagilini lung B cell gene set selected in the bottom panel.

FIG. 11 shows the number of tumour samples in each cancer type histology in the 100KGP data analysed in an example of use of the methods described herein.

FIG. 12 shows the results of analysis of the 100KGP data of FIG. 11 using methods described herein. A-B show the landscape of IGH class switching in the 100KGP pan cancer cohort of FIG. 11, for A. tumour samples, and B. matched germline blood samples. C. Pan cancer distributions for blood and tumour immune fractions; D. Pearson correlations between matched blood and tumour immune fractions for different cancer histologies.

FIG. 13 shows the prognostic value of different IGH B cell classes A. 5 year survival Kaplan-Meier plots of entire pan cancer 100KGP cohort using blood or tumour IGH B cell fraction. High and low groups selected using the median value across the entire cohort; B. Results from Cox proportional hazard models accounting for the effect of age, sex, ancestry and pre-treatment chemotherapy. Left panels: Hazard ratios for different B cell fractions. Right panels: Heatmap of hazard ratios from CoxPH model fitted to different cancer histologies. Hazard ratios and P values per CoxPH model are shown.

FIG. 14 shows the results of an investigation into cancer related disruption of blood immune fraction. A. Scatter plots of date-matched blood count data versus IGH B cell fraction for lymphocyte, neutrophil count and NLR as well as albumin count. B. Blood IGH B cell fraction by sex in 100KGP normal and pan cancer cohort.

FIG. 15 shows an overview of validation analyses performed using embodiments of the disclosure including copy number correction. The heatmap shows calculated fractions from TRACERx100 WGS and TCRA T cell fraction from T Cell ExTRECT from TRACERx100 WES data versus the T and B cell related Danaher scores from matched RNAseq samples.

FIG. 16 shows further validation of the methods of the disclosure including copy number correction. The heatmap shows calculated fractions versus RNAseq signatures for a range of cell types coloured by Spearman Correlation Coefficient.

FIG. 17 shows the result of voom (limma package) dividing TRACERx RNAseq samples into high and low groups based on the median value of predicted non-class switched igM/D B cell fractions and class switched igA and igG B cell fractions. Red points show those genes within the Travagilini lung B cell gene signature, with P values derived from a GSEA analysis (top panel) of all cell type signature gene sets defined by MSigDB.

FIG. 18 shows RNAseq validation for IGH in TRACERx and scRNA data. A-C. Correlation of proportion of RNA reads mapping to IGH from the TRUST algorithm with different class switched proportions to the proportion of class switched B cells as measured from DNA with the present method in TRACERx100. D-F. Correlation of the number of unique IGHV clones as measured by MiXCR from RNAseq data to sample matched B cell fraction as measured by WGS for either IgM/D, IgG or IgA B cells. G. Class switched status of B cell subsets in scRNA Wu et al. data. H. Number of reads by class switch status of different B cell subsets.

FIG. 19 shows the results of nested downsampling of TRACERx data. A. Correlation of circulating B cell fraction at 30× with samples downsampled to different coverage levels using nested downsampling. Top panels: B cell fraction calculated without IGH haplotype correction, Bottom panels: B cell fraction calculated with IGH haplotype correction. B. Correlation of infiltrating B cell fraction at 30× with samples downsampled to different coverage levels using nested downsampling. Top panels: B cell fraction calculated without IGH haplotype correction, Middle panels: B cell fraction calculated with germline IGH haplotype correction. Bottom panels: B cell fraction calculated with germline and somatic IGH haplotype correction.

FIG. 20 shows the performance of the present method on matched high and low coverage data. Scatter plots for IGH B cell fraction calculated with (B.) or without (A.) haplotype correction on the high coverage PCAWG data versus lowpass TCGA. C. IGH B cell fraction calculation on the high coverage 1000 genome data (median coverage 34×) versus the matched low coverage data (1.25×), points are coloured by calculated TCRA T cell fraction from the high coverage data (left panel) and the low coverage data (right panel). D. Scatter plots for IGH class switching fractions for high versus low WGS data with samples with <0.5 IGH B cell fraction in the low coverage cohort removed.

FIG. 21 shows a pan cancer overview of IGH and T/B cell ratio. A. Overview snake plot of age-adjusted TCRA T cell fraction, Circulating and Infiltrating IGH B cell fraction and circulating T/B cell ratio. B. Landscape of IGH class switching in the 100KGP pan cancer cohort. Upper panel: tumour samples, lower panel: matched germline blood panels.

FIG. 22 shows a comparison of circulating and infiltrating fractions. A. Boxplots showing different fractions calculated using the present tool in circulating blood and infiltrating tumour samples, pie charts show the percentage of cases when infiltrating fractions is higher or lower than circulating. B. Pearson correlation of infiltrating and circulating fractions within the 100KGP cohort.

FIG. 23 shows the results of the interpretation of blood TCRA T cell fraction and its disruption in cancer. A. TCRA T cell fraction, IGH B cell fraction and T/B cell ratio versus date-matched blood count data within the 100KGP cohort showing correlations with lymphocyte count, neutrophil count and NLR values. B. Boxplots of distribution of calculated fraction in age deciles split by normal and pan cancer cohort. C. Density plot showing differences in blood TCRA T cell fraction between males and females in either normal or pan-cancer 100KGP cohort. D. Left panels: fold change of female versus males for calculated fraction for both circulating and tumour infiltrating fractions, with p values obtained from a bootstrapping method. Right panels: ratio of calculated circulating fraction from cancer patients with propensity matched for age and sex 100KGP participants within the normal cohort.

FIG. 24 shows the results of investigation into additional determinants of fractions calculated by the present method. A. Calculated fractions versus date-matched blood count data for albumin, C reactive protein, ferritin, platelets and white blood count. B. Fold change with 95% confidence intervals for (left panel) circulating blood fractions in cancer participants with propensity matched normal participants, and (right panel) females versus males.

FIG. 25 shows the result of investigation into disruption of T/B cells in cancer. Density plots shows calculated fractions from a subset of normal cohort with a record of cancer incidence post WGS sequencing from hospital episode statistic, compared to a propensity matched for age and sex cohort of the same size.

FIG. 26 shows the result of analysis of the prognostic ability of cell fractions. The results show that blood TCRA T cell fraction is more prognostic of survival than tumour T cell fraction. A. 5 year survival Kaplan-Meier plots of the entire pan cancer 100KGP cohort using blood or tumour TCRA T cell fraction and IGH B cell fraction. High and low groups selected using the median value across the entire cohort. B. Results from Cox proportional hazard models accounting for the effect of age, sex, ancestry and pre-treatment chemotherapy and stage for participants within the 100KGP cancer cohort with complete clinical annotation.

FIG. 27 shows the results of investigations into the inheritance patterns of IGH B cell fraction enrichment. A. Histogram showing the distribution of circulating IGH B cell fraction in the 100KGP normal or pan cancer cohort, separated by those samples with physiologically normal fractions (<0.1), enriched (>0.1 and <0.2) and high fractions (>0.2). B. Density plots of circulating IGH B cell fraction for the child within a mother-father-child trio, grouped by the status of the parents calculated IGH B cell fraction into physiologically normal, enriched or high groups. C. Left panels: Normalised number of reads in 1 kb bins within the IGH loci for a mother-father-child trio within the 100KGP. 1 kb bins are coloured by the called copy number status as described in the methods disclosed herein, genomic regions showing clear inheritance patterns are highlighted. Right panels: Output of the present method on non-corrected IGH coverage values. D. Normalised number of reads in 1 kb bins for TRACERx patient CRUK0062, germline blood sample and tumour region 4. E. Output of the present method on uncorrected coverage values of CRUK0062 tumour region 4. F. Cartoon overview of method to call germline IGH haplotype copy number alterations within the IGH loci and potential somatic CNA occurring in cancer cells. G. Histogram showing the distribution of circulating IGH B cell fraction in the 100KGP cohort using corrected coverage values for germline IGH haplotype variation called by the present method. H. Output of the present method on corrected IGH coverage values on the mother-father-child trio in C. and CRUK0062 tumour region 4. I. Output of the method of circulating IGH B cell fraction on uncorrected IGH coverage values for a participant that has B cell chronic lymphocytic leukaemia.

FIG. 28 shows the result of IGH B cell fraction validation using the LCL 1000 genomes cohort. A. Histogram distribution of IGH B cell fraction in the 1000 genome cohort. B. Scatter plot of IGHV Shannon diversity vs IGH B cell fraction. C. Distribution of the predicted percentage of B cells that have undergone allelic exclusion in the 1000 genomes cohort. D. Scatter plot of the IGHV Shannon diversity index as a function of the predicted percentage of B cells with allelic exclusion. E. Output calculated using the present tool for IGH B cell fraction of three samples within the 1000 genome LCL cohort. F. Volcano plots of limma voom analysis of the Geuvadis 1000 genome RNAseq data with samples separated into high and low by the median of the igM/D, igG or igA B cell fractions.

### DETAILED DESCRIPTION

The specific embodiments described herein are offered by way of example, not by way of limitation. Various modifications and variations of the described methods, devices, systems and uses of the technology will be apparent to those skilled in the art without departing from the scope and spirit of the technology as described.

Any sub-titles herein are included for convenience only, and are not to be construed as limiting the disclosure in any way.

The methods of any embodiments described herein may be provided as computer programs or as computer program products or non-transitory computer readable media carrying a computer program (i.e. a set of instructions) which is configured, when run on a computer, to perform the method(s) described above.

Unless context dictates otherwise, the descriptions and definitions of the features set out below are not limited to any particular aspect or embodiment of the invention and apply equally to all aspects and embodiments which are described. Throughout the specification and claims, the following terms take the meanings explicitly associated herein, unless the context clearly dictates otherwise. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. Other aspects and embodiments of the invention provide the aspects and embodiments described herein with the term “comprising” replaced by the term “consisting of” or “consisting essentially of”, unless the context dictates otherwise. The features disclosed in the present description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%. Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

A “sample” as used herein may be a cell or tissue sample (e.g. a biopsy), a biological fluid, an extract (e.g. a DNA extract obtained from the subject), from which genomic material can be obtained for genomic analysis, such as genomic sequencing (whole genome sequencing, whole exome sequencing, targeted (also referred to as “panel” sequencing) or copy number array profiling. The sample may be a cell, tissue or biological fluid sample obtained from a subject (e.g. a biopsy). Such samples may be referred to as “subject samples”. In particular, the sample may be a blood sample, or a tumour sample, or a sample derived therefrom. The sample may be one which has been freshly obtained from a subject or may be one which has been processed and/or stored prior to genomic analysis (e.g. frozen, fixed or subjected to one or more purification, enrichment or extractions steps). In particular, the sample may be a cell or tissue culture sample. As such, a sample as described herein may refer to any type of sample comprising cells or genomic material derived therefrom, whether from a biological sample obtained from a subject, or from a sample obtained from e.g. a cell line. The sample is preferably from a jawed vertebrate (such as e.g. a jawed vertebrate cell sample or a sample from a jawed vertebrate subject), suitably from a mammalian (such as e.g. a mammalian cell sample or a sample from a mammalian subject, including in particular a model animal such as mouse, rat, etc.), preferably from a human (such as e.g. a human cell sample or a sample from a human subject). In embodiments, the sample is a sample obtained from a subject, such as a human subject. Further, the sample may be transported ad/or stored, and collection may take place at a location remote from the genomic sequence data acquisition (e.g. sequencing) location, and/or the computer-implemented method steps may take place at a location remote from the sample collection location and/or remote from the genomic data acquisition (e.g. sequencing) location (e.g. the computer-implemented method steps may be performed by means of a networked computer, such as by means of a “cloud” provider).

A sample is typically a “mixed sample”, which refers to a sample that is assumed to comprise multiple cell types or genetic material derived from multiple cell types. Samples obtained from subjects, such as e.g. tumour samples, are typically mixed samples (unless they are subject to one or more purification and/or separation steps). Preferably, a mixed sample is one that comprises B lymphocytes, or is assumed to (expected to) to comprise B lymphocytes. Suitably, the sample comprises B lymphocytes and at least one other cell type. For example, the sample may be a tumour sample. A “tumour sample” refers to a sample derived from or obtained from a tumour. Such samples may comprise tumour cells, immune cells (such as e.g. lymphocytes), and other normal (non-tumour) cells. In the context of a tumour sample, the term “purity” refers to the proportion of cells in the sample that are tumour cells (also sometimes referred to as “cancer cell fraction” or “tumour fraction”), or to the equivalent proportion of cells in the case of a sample comprising genetic material derived from cells. In the context of samples comprising genetic material, a tumour fraction may be estimated using sequence analysis processes that attempt to deconvolute tumour and germline genomes such as e.g. ASCAT (Van Loo et al., 2010), etc. In the context of tumour samples, the lymphocytes in the sample may be referred to as “tumour infiltrating lymphocytes” (TIL). A tumour sample may be a primary tumour sample, tumour-associated lymph node sample, or a sample from a metastatic site from the subject. A sample comprising tumour cells or genetic material derived from tumour cells may be a bodily fluid sample. Thus, the genetic material derived from tumour cells may be circulating tumour DNA or tumour DNA in exosomes. Instead or in addition to this, the sample may comprise circulating tumour cells. A sample may be a sample of cells, tissue or bodily fluid that has been processed to extract genetic material. Methods for extracting genetic material from biological samples are known in the art.

The term “lymphocyte fraction” refers to the proportion of DNA containing cells within a mixed sample that are lymphocytes, more precisely B lymphocytes. The term “B lymphocyte fraction” refers to the proportion of DNA containing cells within a sample that are B lymphocytes. Within the context of the present disclosure, a B lymphocyte fraction is estimated based on a signal derived from a genomic region that undergoes VDJ recombination in the IGH locus, in the particular lymphocyte population that is being quantified. Thus, the lymphocyte fraction may refer more particularly to the proportion of DNA containing cells within a sample that are lymphocytes characterised by having a predetermined genomic region in the IGH gene that has undergone VDJ recombination. Using the IGH locus may allow the quantification of B cells (regardless of whether they have the IGL or IGK light chains. Further, separate lymphocyte fractions (e.g. for each of a plurality of B cell classes) may be quantified for multiple subpopulations of lymphocytes. These may be added to obtain a lymphocyte fraction that is representative of multiple or all subpopulations of B lymphocytes.

The IGH gene refers to the immunoglobulin heavy locus with Gene ID 3492 (HGNC symbol: IGH) in Homo sapiens, or any homologous region in another jawed vertebrate. In Homo sapiens, this gene is located on Chromosome 14, at location NC_000014.9 (105586437 . . . 106879844, complement) (GRCh38.p13) or NC_000014.8 (106032614 . . . 107288051, complement) (GRCh37.p13). Examples of homologous loci include the mouse Igh locus (gene ID: 11507, located on Chromosome 12, assembly GRCm39—NC_000078.7 (113222388 . . . 115973574, complement), assembly GRCm38.p6—NC_000078.6 (113258768 . . . 116009954, complement)).

The term “sequence data” refers to information that is indicative of the presence and preferably also the amount of genomic material in a sample that has a particular sequence. Such information may be obtained using sequencing technologies, such as e.g. next generation sequencing (NGS, such as e.g. whole exome sequencing (WES), whole genome sequencing (WGS), or sequencing of captured genomic loci (targeted or panel sequencing)), or using array technologies, such as e.g. copy number variation arrays, or other molecular counting assays. When NGS technologies are used, the sequence data may comprise a count of the number of sequencing reads that have a particular sequence. Sequence data may be mapped to a reference sequence, for example a reference genome, using methods known in the art (such as e.g. Bowtie (Langmead et al., 2009)). Thus, counts of sequencing reads or equivalent non-digital signals may be associated with a particular genomic location (where the “genomic location” refers to a location in the reference genome to which the sequence data was mapped). The term “read depth” refers to a signal that is indicative of the amount of genomic material in a sample that maps to a particular genomic location. Such a signal may be obtained using sequencing technologies, such as e.g. next generation sequencing (NGS, such as e.g. WES, WGS, or sequencing of captured genomic loci), or using array technologies, such as e.g. copy number variation arrays. When NGS technologies are used, a read depth may be a read depth within the common sense of the word, i.e. a count of the number of sequencing reads mapping to a genomic location. When array technologies are used, a read depth may be an intensity value associated with a particular genomic location, which can be compared to a control to provide an indication of the amount of genomic material that maps to the particular location. The term “read depth profile” refers to a collection of read depth values relating to a plurality of genomic locations. For example, a read depth at a particular genomic location i may refer to the read depth at the base at position i in a reference genome, and a read depth profile may refer to the read depth for a plurality of positions i within one or more regions of interest.

As used herein “treatment” refers to reducing, alleviating or eliminating one or more symptoms of the disease which is being treated, relative to the symptoms prior to treatment. “Prevention” (or prophylaxis) refers to delaying or preventing the onset of the symptoms of the disease. Prevention may be absolute (such that no disease occurs) or may be effective only in some individuals or for a limited amount of time.

As used herein, the terms “computer system” includes the hardware, software and data storage devices for embodying a system or carrying out a method according to the above described embodiments. For example, a computer system may comprise a processing unit (such as a central processing unit (CPU) and/or graphical processing unit (GPU)), input means, output means and data storage, which may be embodied as one or more connected computing devices. Preferably the computer system has a display or comprises a computing device that has a display to provide a visual output display (for example in the design of the business process). The data storage may comprise RAM, disk drives or other computer readable media. The computer system may include a plurality of computing devices connected by a network and able to communicate with each other over that network. It is explicitly envisaged that computer system may consist of or comprise a cloud computer.

As used herein, the term “computer readable media” includes, without limitation, any non-transitory medium or media which can be read and accessed directly by a computer or computer system. The media can include, but are not limited to, magnetic storage media such as floppy discs, hard disc storage media and magnetic tape; optical storage media such as optical discs or CD-ROMs; electrical storage media such as memory, including RAM, ROM and flash memory; and hybrids and combinations of the above such as magnetic/optical storage media.

### Determining the Class-Specific B Lymphocyte Fraction in a Sample

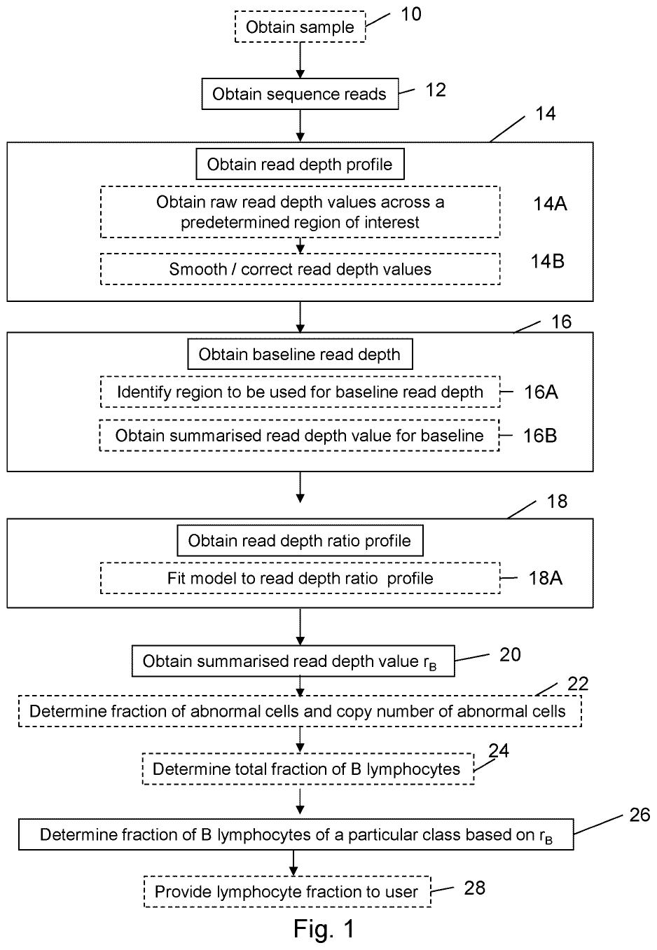

The present disclosure provides method for determining class-specific B lymphocyte fractions in a sample comprising cells or genetic material from cells from different cell types and/or different classes of B cells, using read depth data from the sample comprising read depth data for at least a predetermined genomic region comprising parts of the IGH locus. An illustrative method will be described by reference to FIG. 1. At optional step 10, a sample comprising genomic material from multiple cell types may be obtained from a subject. At step 12, sequence data comprising a plurality of sequencing reads is obtained from the sample, for example by sequencing the genomic material in the sample using one of whole exome sequencing, whole genome sequencing, or panel sequencing. In embodiments, the sequence data is obtained from a data store or user interface, and has been previously acquired from the sample. At step 14, a read depth profile for the sample is derived from the sequence data, comprising the read depths along a predetermined genomic region, wherein the predetermined genomic region includes at least a region of the IGH locus that undergoes class switch recombination and a region of the IGH locus that does not undergo class switch recombination. The predetermined genomic region may further include a region that undergoes VDJ recombination. Step 14 may comprise step 14A of obtaining a plurality of raw read depth values across the predetermined genomic region and step 14B of smoothing and/or correcting (e.g. GC correction, somatic and/or germline copy number alteration correction) the read depth values.

The read depth or read depth ratios may be processed to correct for biases associated with the read depth data acquisition platform used. For example, the read depth/read depth ratios may be GC corrected. GC correction of read depth data may be performed using a linear model capturing the influence of GC content on read depth at every position, where the residuals of the model represent the normalised read depth values. The influence of GC content on read depth may be represented by a term that reflects GC content at the exon or region level (GCₑₓₒₙ/GC_{region}) where a region may be defined as a window of a predetermined size such as e.g. 1000 bp (in which case GC_{region} may be referred to as “GC_{1000bp}”) and a term that reflects the GC content at the level of the entire predetermined region of interest (GC_{macro}, obtained from a model, for example a linear model, fitted to smoothed data such as for example using 1000 bp windows along the predetermined region of interest). For example, a linear model of the form Im(basepair~GCₑₓₒₙ+GC²ₑₓₒₙ+GC_{macro}+GC²_{macro}) or equivalently Im(basepair~ GC_{region}+GC²_{region}+GC_{macro}+GC²_{macro}) (such as in particular Im(basepair~ GC_{1000bp}+GC²_{1000bp}+GC_{macro}+GC²_{macro})) may be used, where “basepair” is the read depth at every position. The residuals of such a model may be used as the corrected coverage values.

The corrected (e.g. GC corrected) read depth values may be used to determine the baseline read depth derived from a subset of the region of interest and the summarised read depth ratio value (r_{VDJ}) for the subset of the region of interest that is likely to be deleted through VDJ recombination. For example, the median value (or other summarised metric such as e.g. a statistical measure of centrality) of the corrected values at the respective subset of the region of interest may be used. Where the subset of the region of interest comprises a plurality of genomic locations (such as e.g. the beginning and end of the region likely to undergo VDJ recombination), one of the plurality of median values may be used, such as e.g. the highest one. A read depth profile may be obtained from sequencing data, preferably from whole exome sequencing, whole genome sequencing (including low pass WGS, also referred to as shallow genome sequencing), or panel sequencing data. When the read depth profile is obtained from panel sequencing data, the panel sequencing data comprises data for the predetermined genomic region. For example, the data may have been obtained using capture probes that target a plurality of genomic regions including the predetermined genomic region.

Obtaining a read depth profile for the sample may comprise obtaining raw read depth values from the sequence data in the predetermined genomic region and correcting said raw read depth values (or said read depth values corrected for GC content) for copy number alterations in the predetermined region. Correcting raw or GC corrected read depths values for copy number alterations in the predetermined regions may comprise correcting for germline copy number alterations (e.g. in the case of a germline sample). Correcting raw or GC corrected read depths values for copy number alterations in the predetermined regions may comprise correcting for germline copy number alterations and somatic copy number alterations (e.g. for some tumour samples).

In embodiments where the sample is a germline sample, obtaining a read depth profile for the sample may comprise obtaining read depth values from the sequence data in the predetermined genomic region corrected for germline copy number alterations in the predetermined region by: dividing the raw read depth values in the predetermined region, optionally the IGH locus, by the median read depth in the predetermined region, smoothing the resulting read depth values using a rolling average over windows of a predetermined size (e.g. 1000 bp) along the predetermined genomic region, rounding the resulting read depth values to the nearest 0.5 value over windows of a predetermined size along the predetermined genomic region, and setting the raw read depths to zero in regions where the rounded read depth values indicate a loss of the region on both alleles, and dividing the raw read depth values by 0.5 in regions where the rounded read depth values indicate the loss of the region in one allele, by 1.5 in regions where the rounded read depth values indicate the gain of the region in one allele, and by 2 in regions where the rounded read depth values indicate a duplication of the region.

Such a method may be particularly useful in the context of germline samples with low predicted B cell fraction (e.g. predicted B cell fraction below 10%), such as e.g. blood samples. For samples with higher predicted B cell fraction (e.g. lymphoblastic cell line samples), obtaining a read depth profile for the sample can comprises obtaining read depth values from the sequence data in the predetermined genomic region corrected for germline copy number alterations in the predetermined region by: dividing the raw read depth values in the predetermined region, optionally the IGH locus, by the median read depth in a region of the predetermined region that is not expected to be affected by V(D)J recombination, optionally the last 20 kb of the IH locus; obtaining a smoothed and rounded read depth value over windows of a predetermined size along the predetermined genomic region; identifying locations where there is a drop and then rise in coverage when moving towards the end of the predetermined region, said drop then increase corresponding to breakpoints of a region with copy number alteration; and setting the raw read depths to zero in regions where the rounded read depth values indicate a loss of the region on both alleles, and dividing the raw read depth values by 0.5 in regions where the rounded read depth values indicate the loss of the region in one allele, by 1.5 in regions where the rounded read depth values indicate the gain of the region in one allele, and by 2 in regions where the rounded read depth values indicate a duplication of the region.

In embodiments where the sample is a tumour sample, obtaining a read depth profile for the sample can comprise obtaining read depth values from the sequence data in the predetermined genomic region corrected for somatic copy number alterations in the predetermined region by: identifying one or more regions with a somatic copy number alteration in the IGH locus using the read depth profile for the sample and a read depth profile from a matched germline sample, and dividing the read depths within the one or more regions identified as having a somatic copy number alteration by the ratio of the median read depth value within the region to the median read depth value across the IGH locus,

Identifying somatic copy number alterations in the IGH locus using the read depth profile for the sample and a read depth profile from a matched germline sample can comprise: obtaining a GC corrected read depth profile for the sample and a GC corrected read depth profile from the matched germline sample; normalising each of said read depth profiles by dividing read depth values by the respective median value across the IGH locus; obtaining a log R profile as the log 2 ratio of the normalised read depth profiles for the tumour and germline samples; and identifying segments in said log R profile, optionally by obtaining median values for each of a plurality of windows of predetermined size (e.g. 1000 bp) and performing recursive partitioning to identify segments (e.g. by fitting a recursive partitioning and regression tree), wherein a region identified as having a somatic copy number alteration is a region associated with a segment of a length above a predetermined threshold (e.g. 100 kb) that has a log R different from 1.

In embodiments, obtaining read depth values from the sequence data in the predetermined genomic region corrected for somatic copy number alterations in the predetermined region is performed when the maximum log R deviation from 1 across the segments identified is above a predetermined threshold. The predetermined threshold may be e.g. 0.1. This ensures that somatic copy number correction is only performed when there is evidence of significant copy number alteration. The steps of identifying segments in the log R profile may be performed over the IGH locus excluding read depths in one or more predetermined regions that are associated with class switching and/or one or more predetermined regions that are associated with V(D)J recombination. The method may further comprise identifying a segment that overlaps an excluded region and that has a log R value not equal to 1, and repeating the step of identifying segments in a log R profile for the region corresponding to the segment including the excluded region overlapped by the segment, thereby identifying one or more subsegments. The method may further comprise selecting a breakpoint between two subsegments as the breakpoint associated with the segment in the excluded region when the breakpoint is associated with a log R deviation from 1 that differs from the log R deviation of the segment by less than a predetermined threshold.

In some such embodiments, obtaining a read depth profile for the sample can comprise: obtaining read depth values from the sequence data in the predetermined genomic region corrected for somatic copy number alterations in the predetermined region; and obtaining read depth values from the sequence data in the predetermined genomic region further corrected for germline copy number alterations in the predetermined region. Obtaining read depth values from the sequence data in the predetermined genomic region further corrected for germline copy number alterations in the predetermined region can be performed by identifying regions associated with a germline copy number alteration using a read depth profile from a matched germline sample; and dividing the read depths values corrected for somatic copy number alterations on regions associated with a germline copy number alteration by the ratio of the median read depth within the region in the tumour sample and the median read depth across the IGH locus in the tumour sample. In some such embodiments, a fraction of B lymphocytes in the sample is obtained using the read depths values corrected for germline copy number alterations using the method of claim 20, and using the read depth values corrected for germline and somatic copy number alterations, and the read depth values associated with the lowest fraction of B lymphocytes in the sample are used for determining the fraction of B lymphocytes of a particular class in the sample.

Identifying regions associated with a germline copy number alteration using a read depth profile from a matched germline sample can be performed as explained above, i.e. by dividing the raw read depth values from the germline sample in the predetermined region, optionally the IGH locus, by the median read depth in the predetermined region in the germline sample, smoothing the resulting read depth values using a rolling average over windows of a predetermined size along the predetermined genomic region, and rounding the resulting read depth values to the nearest 0.5 value over windows of a predetermined size along the predetermined genomic region. This rounded read depth profile can then be used to identify regions where the rounded read depth values indicate the loss of the region in one allele in the germline sample, regions where the rounded read depth values indicate the gain of the region in one allele in the germline sample, and regions where the rounded read depth values indicate a duplication of the region in the germline sample.

At optional step 16, a baseline read depth is obtained from the region of the IGH locus that does not undergo class switch recombination. This may comprise optional step 16A of identifying a region of the IGH locus that does not undergo class switch recombination that can be used to obtain the baseline read depth. The region that identified at step 16A may comprise a plurality of individual genomic segments. The one or more genomic segments may be chosen as regions of the IGH locus of regions proximal to the IGH locus. Preferably, the segments are regions of the IGH locus that do not undergo class switch recombination or VDJ recombination. For example, a region of the IGH locus that does not undergo class switch recombination may comprise a region of the IGH locus that is located before segments that can be deleted through class switching, and/or a region of the IGH locus that is located after segments that can be deleted through VDJ recombination. A region that is unlikely to be deleted through VDJ recombination (or CSR) may comprise the last n V segments of the IGH locus. The parameter n may be determined using appropriate training data, for example by choosing a parameter n that results in the most accurate estimate of total B lymphocyte fraction across the training data, where accuracy of the estimate is determined by comparing the estimate obtained using a method as described herein to an orthogonal metric of B lymphocyte fraction. An orthogonal metric of B lymphocyte fraction may be obtained for examples based on transcriptomic data and/or histopathology data. A region that is unlikely to be deleted through VDJ recombination or CSR may comprise a region before the first segment that is deleted through CSR, such as e.g. a region before the class switching segment IGHA2, for example a genomic segment from the start of the IGH locus to the start of the first class switching segment IGHA2. Step 16 may further comprise step 16B of obtaining a summarised read depth value, such as e.g. a median read depth, across the region identified at step 16A.

At step 18, a plurality of read depth ratios (rᵢ) are obtained by normalising the read depths along the region of the IGH locus that undergoes class switch recombination, and optionally the region that undergoes VDJ recombination, by reference to the baseline read depth obtained at step 16. For example, each read depth value (including a read depth value for each base or group of bases, when the read depth values are calculated for bins along genomic coordinates) may be divided by the baseline read depth value obtained at step 16. Further, the read depth ratios may be transformed using a log transform (such as e.g. a log base 2). Step 18 may comprise optional step 18A of fitting a model to the read depth ratio profile (comprising the plurality of read depth ratios (rᵢ)), or a plurality of individual models for different genomic regions, to obtain a smoothed read depth profile. The model(s) may each be selected from: a general linear model, a piecewise constant linear model with a plurality of breakpoints selected to correspond to locations of a plurality of segments in the modelled region of the IGH locus, or a Bayesian model modelling segment usage from the read depth ratios and a prior distribution of usage of each of a plurality of segments in the modelled region of the IGH locus. Separate models may be fitted for: (a) a region of the IGH locus that undergoes VDJ recombination, and (b) a region of the IGH locus that undergoes class switch recombination. Each model may be fitted using one or more constraints. When a plurality of models are fitted, the models may have different constraints. A model fitted for a region of the IGH locus that undergoes class switch recombination may be a piecewise linear model fitted with constraints including: breakpoints between constant sections are restricted to locations of the ends of a plurality of segments in the constant region of the IGH locus, the read depth ratio value at the end of the IGHM segment in the IGH locus must be 0, and the read depth ratio after the IGHG3 segment in the IGH locus must be less or equal to the read depth ratio for a subset of the region of the IGH locus that is likely to be deleted through VDJ recombination. A model fitted for a region of the IGH locus that undergoes VDJ recombination may be a piecewise linear model fitted with constraints including: breakpoints between constant sections are restricted to locations of the ends of a plurality of V and J segments in the IGH locus, the read depth ratio value before the first V segment must be 0, the read depth ratio value must decrease monotonically between the first V segment and the last V segment, the read depth ratio value must increase monotonically between the first J segment and the last J segment, and the read depth ratio value after the last J segment must be 0.

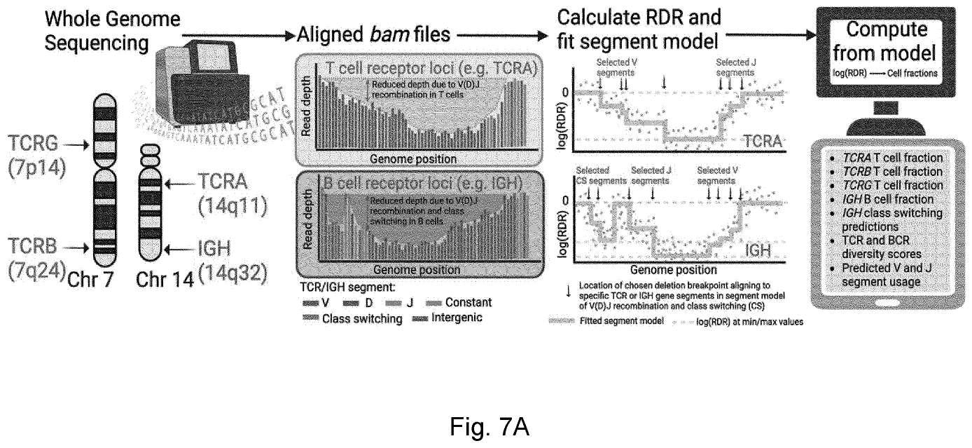

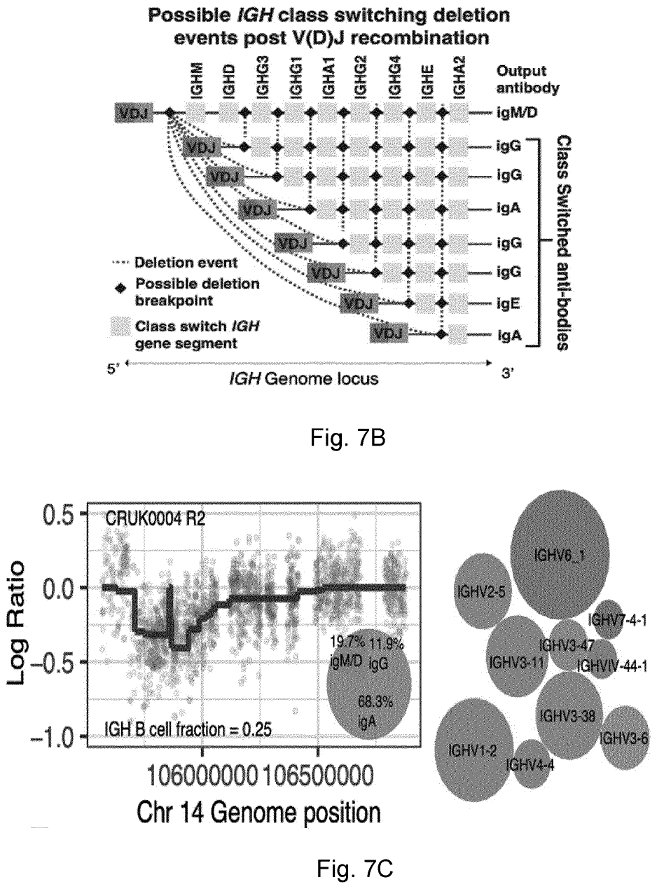

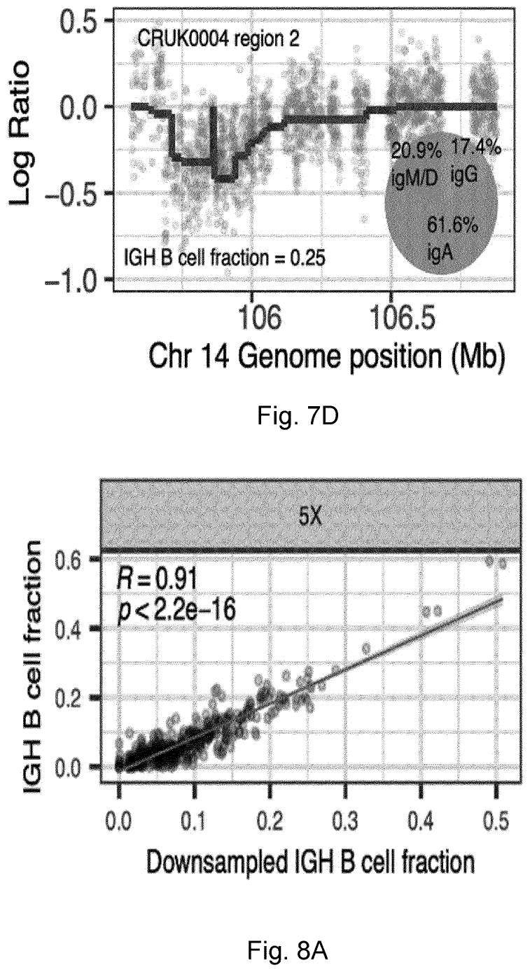

At step 20, one or more summarised read depth ratio value(s) (r_{B}) is/are obtained for the region of the IGH locus that undergoes class switch recombination (optionally based on the value of the model fitted at step 18A, in the region of the IGH locus that undergoes class switch recombination). For example, a summarised read depth ratio value (r_{B}) for a portion of the IGH locus comprising both the IGHD segment and the IGHM segment may be obtained. Such a region may be referred to as a region of maximum class switch recombination. Such a region may be expected to be deleted in all class switched B cells. Therefore, such a summarised read depth may be used to determine the fraction of class switched B cells. Such a summarised read depth value may be obtained as the value of the model fitted at step 18A (in the case of a piecewise linear model that comprises a segment corresponding to the IGHD and IGHM segments) or a summarised value of the model fitted at step 18A (e.g. a median or average value of the model, for example in the case of a generalised linear model, over a region corresponding to the IGHD and IGHM segments). Instead or in addition to this, one or more summarised read depth ratio value(s) (r_{B}) is/are obtained for respective segments in the region of the IGH locus that undergoes class switch recombination, wherein the summarised read depth ratio for a particular segment is the absolute value of the difference between the read depth value for the particular segment of the region that undergoes CSR and the read depth value for the preceding segment (where segments order is as indicated on FIG. 7B, i.e. IGHM, IGHD, IGHG3, IGHG1, IGHA1, IGHG2, IGHG4, IGHE, IGHA2). The read depth value for a segment may be the value of a model fitted at step 18A (when the model is a piecewise linear model with breakpoints corresponding to said segments), or a summarised value derived from the model fitted at step 18A over a region corresponding to the respective segments (e.g. the median or average value of the model over a segment). The summarised read depth ratio for a segment calculated as the absolute value of the difference between the read depth value for the particular segment of the region that undergoes CSR and the read depth value for the preceding segment can be used to calculate the fraction of B lymphocytes that have a deletion breakpoint between the two segments (i.e. that have a deletion of the particular segment). As illustrated on FIG. 7B, the fraction of cells that have a deletion breakpoint before e.g. segment IGHE is indicative of the fraction of cells that have segments IGHM to IGHG4 deleted, preserving only segments IGHE and IGHA2. These cells are igE cells. Similar principles apply to all segments, i.e. the difference between the read depth ratio for a segment (e.g. IGHE) and a preceding segment (e.g. IGHG4) is indicative of the fraction of cells that have a deletion from the IGHE segment to the end of the CSR portion of the IGH locus (i.e. segments IGHG4 to IGHM.)

At optional step 22, the fraction of abnormal cells (p) in the mixed sample, where abnormal cells are those that are aneuploid in the region of the IGH locus, and the copy number of the abnormal cells in the region of the IGH locus (ψ_{T}) are determined by obtaining an abnormal cell-specific copy number profile using methods known in the art.

At optional step 24, the total fraction of B lymphocytes in the sample is determined. Step 24 may comprise step 24A of obtaining a further summarised read depth ratio value for a subset of the region of the IGH locus that is likely to be deleted through VDJ recombination (optionally based on the value of the model fitted at step 18A, in the region of the IGH locus that undergoes VDJ recombination). Step 24 may comprise step 24B of determining the total fraction of B lymphocytes (f^{tot}) in the sample as a function of the further summarised read depth ratio value (r_{B}), in a similar way as described below for step 26, such as e.g. using equation (7) or equation (3).

At step 26, the fraction of B lymphocytes of a particular class in the sample is determined as a function of the summarised read depth ratio value (r_{B}) obtained at step 20. This may comprise determining the fraction of B lymphocytes of a particular class in the sample as a function of the summarised read depth ratio value (r_{B}), the fraction of abnormal cells (p) in the mixed sample and the copy number of the abnormal cells in the region of the IGH locus (ψ_{T}), for example using equation (7) described below. Alternatively, step 26 may comprise determining the fraction of B lymphocytes in a particular class as a function of the summarised read depth ratio value (r_{B}), for example using equation (3). This may be particularly useful in embodiments where the sample is not expected to comprise a significant proportion of cells that are not expected to be diploid in the region of the IGH locus. The particular class of B lymphocytes is selected from: igG B cells, igA B cells, igE B cells, igM/D B cells and combinations thereof, such as e.g. class-switched B cells (comprising all B cells in any of classes IgG B cells, igA B cells, and igE B cells), and/or all non-class switched B cells (comprising B cells in class igM/D B cells).

In embodiments, the one or more summarised read depth ratio values (r_{B}) obtained at step 18 comprise summarised read depth ratio values (r_{B}) for respective portions of the region of the IGH locus each comprising a segment selected from IGHG1, IGHG2, IGHG3, and IGHG4. In some such embodiments, a fraction of B lymphocytes in the igG B class is calculated at step 26 as the sum of: a fraction of B lymphocytes that include a deletion breakpoint before the IGHG1 segment (obtained using a summarised read depth value for the IGHG1 segment), a fraction of B lymphocytes that include a deletion breakpoint before the IGHG2 segment (obtained using a summarised read depth value for the IGHG2 segment), a fraction of B lymphocytes that include a deletion breakpoint before the IGHG3 segment (obtained using a summarised read depth value for the IGHG3 segment), and a fraction of B lymphocytes that include a deletion breakpoint before the IGHG4 segment (obtained using a summarised read depth value for the IGHG4 segment). In embodiments, the one or more summarised read depth ratio values (r_{B}) obtained at step 18 comprise summarised read depth ratio values (r_{B}) for respective portions of the region of the IGH locus each comprising a segment selected from IGHA1 and IGHA2. In some such embodiments, a fraction of B lymphocytes in the igA B class is calculated at step 26 as the sum of: a fraction of B lymphocytes that include a deletion breakpoint before the IGHA1 segment and a fraction of B lymphocytes that include a deletion breakpoint before the IGHA2 segment. In embodiments, the one or more summarised read depth ratio values (r_{B}) obtained at step 18 comprise a summarised read depth ratio values (r_{B}) for a portion of the region of the IGH locus that comprises a deletion breakpoint before the IGHE segment. In some such embodiments, a fraction of B lymphocytes in the IgE class is calculated at step 26 as the fraction of B lymphocytes that include the a deletion breakpoint before the IGHE segment. In embodiments, the one or more summarised read depth ratio values (r_{B}) obtained at step 18 comprise summarised read depth ratio values (r_{B}) for respective portions of the region of the IGH locus each comprising a segment selected from IGHG1, IGHG2, IGHG3, IGHG4, IGHA1, IGHA2 and IGHE. In some such embodiments, a fraction of B lymphocytes in the igM/D class is calculated at step 26 as the total B lymphocyte fraction calculated at step 24 minus the sum of: the fraction of B lymphocytes that include a deletion breakpoint before the IGHG1 segment, the fraction of B lymphocytes that include a deletion breakpoint before the IGHG2 segment, the fraction of B lymphocytes that include a deletion breakpoint before the IGHG3 segment, the fraction of B lymphocytes that include a deletion breakpoint before the IGHG4 segment, the fraction of B lymphocytes that include a deletion breakpoint before the IGHA1 segment, the fraction of B lymphocytes that include a deletion breakpoint before the IGHA2 segment, and the fraction of B lymphocytes that include a deletion breakpoint before the IGHE segment.

At optional step 28, the determined lymphocyte fraction and/or any value derived therefrom may be provided to a user, for example through a user interface. The value derived therefrom may include prognostic and/or diagnostic information, as described further below.

### Applications

The above methods find applications in a variety of clinical contexts. For example, the present inventors have shown that the proportion of igM/D B lymphocytes in a sample from a subject with cancer (particularly a blood sample) provides an indication of the prognosis for the subject.

Therefore, also described herein are methods of providing a prognosis for a subject that has been diagnosed as having a cancer, the method comprising determining the fraction of B lymphocytes in a particular class (specifically the igM/D class) in one or more samples from the subject. The method may further comprise classifying the sample(s) as naïve B cell high or naïve B cell low depending on the igM/D B lymphocyte fraction in the sample(s). For examples, samples with a igM/D B lymphocyte fraction above a threshold (for example, about 0.1, i.e. about 10%) may be classified as naïve B cell high, whereas samples with a igM/D B lymphocyte fraction at or below the threshold may be classified as naïve B cell low. The first group may have a good prognosis group and the second group may have a poor prognosis. The poor prognosis group may be associated with reduced relapse free survival and/or reduced overall survival, compared to the good prognosis group. A threshold on the igM/D B lymphocyte fraction may be determined using an appropriate training cohort, for example by assessing the ability of various thresholds to classify patients between groups that are associated with significantly different prognosis.

The present methods also find uses in any clinical context where the presence/absence or abundance of specific types of B cells in a sample is indicative of a diagnosis or prognosis. For example, some autoimmune diseases are associated with an increased proportion of class switched (non-naïve) B cells in tissues affected. Thus, also described herein are methods of diagnosing a subject as having a disease, disorder or condition associated with abnormal B lymphocyte counts in a particular class (e.g. abnormal non-class switched, also referred to as “non-naïve” B cells), the method comprising determining the lymphocyte fraction in one or more samples from the subject.

Naïve B cells may also be referred to as “non-class switched” B cells. They may include igG B cells, igA B cells, and igE B cells. Non-naïve B cells may also be referred to as “class-switched” B cells. They may include igM/D B cells.

The presence of class switched B cells in a tissue may be indicative of a loss of B cell tolerance and production of auto-antibodies. Thus, the fraction of class-switched B cells (or conversely, non-class switched B cells) may be used to diagnose and/or monitor auto-immune diseases. For example, in the case of lupus nephritis, kidney biopsies are frequently collected for diagnosis and could be further analysed using the methods described herein. This may be informative of the status of the disease and may also guide treatment of the subject.

In particular, also described herein are methods of diagnosing a subject as having an autoimmune disease or disorder, the method comprising determining the fraction of B lymphocytes of one or more particular classes in a sample from the subject using a method as described herein, wherein the one or more classes are class-switched B cell classes, and comparing the determined fraction(s) to a predetermined threshold (or respective predetermined thresholds, when multiple fractions are used), wherein the subject is diagnosed as having the autoimmune disease if one or more of said determined fractions are above said threshold(s). Also described herein are methods of diagnosing a subject as having an autoimmune disease or disorder, the method comprising determining the fraction of B lymphocytes of one or more particular classes in a sample from the subject using a method as described herein, wherein the one or more classes are non-class-switched B cell classes, and comparing the determined fraction(s) to a predetermined threshold (or respective predetermined thresholds, when multiple fractions are used), wherein the subject is diagnosed as having the autoimmune disease if one or more of said determined fractions are below said threshold(s). The predetermined threshold(s) may be defined using a reference sample or set of samples, such as e.g. samples from a cohort of individuals that are known to have the autoimmune disease and/or samples from a cohort of individuals that are known not to have the autoimmune disease (e.g. healthy subjects). The methods may further comprise treating the subject for the autoimmune disorder by administering a treatment for the autoimmune disorder. Thus, also described herein are methods of treating a subject for an autoimmune disorder, the methods comprising: diagnosing the subject as having the autoimmune disorder using a method as described herein, and administering a treatment specific to the autoimmune disorder that has been diagnosed.

Also described herein are methods of monitoring a subject that has been diagnosed as having or being likely to have an autoimmune disease or disorder, the method comprising determining the fraction of B lymphocytes of one or more particular classes in a sample from the subject using a method as described herein, wherein the one or more classes are class-switched B cell classes, and comparing the determined fraction(s) to a predetermined value (or respective predetermined value, when multiple fractions are used). The predetermined value(s) may be values that have been obtained from analysing a sample of the same subject at a previous time point. The predetermined value(s) may be defined using a reference sample or set of samples, such as e.g. samples from a cohort of individuals that are known to have the autoimmune disease and/or samples from a cohort of individuals that are known not to have the autoimmune disease (e.g. healthy subjects). The method may comprise determining the stage or status of the disease by comparing the determined fraction(s) with the predetermined value(s). The method may comprise determining that the subject's condition has worsened when one or more (or all) of the determined fractions are above the corresponding predetermined values. The method may comprise determining that the subject's condition has improved or remained stable when one or more (or all) of the determined fractions are below or not significantly above the corresponding predetermined values.

Also described herein are methods of monitoring a subject that has been diagnosed as having or being likely to have an autoimmune disease or disorder, the method comprising determining the fraction of B lymphocytes of one or more particular classes in a sample from the subject using a method as described herein, wherein the one or more classes are non-class-switched B cell classes, and comparing the determined fraction(s) to a predetermined value (or respective predetermined value, when multiple fractions are used). The predetermined value(s) may be values that have been obtained from analysing a sample of the same subject at a previous time point. The predetermined value(s) may be defined using a reference sample or set of samples, such as e.g. samples from a cohort of individuals that are known to have the autoimmune disease and/or samples from a cohort of individuals that are known not to have the autoimmune disease (e.g. healthy subjects). The method may comprise determining the stage or status of the disease by comparing the determined fraction(s) with the predetermined value(s). The method may comprise determining that the subject's condition has worsened when one or more (or all) of the determined fractions are below the corresponding predetermined values. The method may comprise determining that the subject's condition has improved or remained stable when one or more (or all) of the determined fractions are above or not significantly below the corresponding predetermined values. The methods may further comprise treating the subject for the autoimmune disorder by administering a treatment for the autoimmune disorder. Thus, also described herein are methods of treating a subject for an autoimmune disorder, the methods comprising: monitoring the subject using a method as described herein, and administering a treatment specific to the autoimmune disorder that has been diagnosed, wherein the treatment administered depends on whether the subject's condition is determined to have improved or remained stable, or has worsened. For example, the subject may be a subject who is undergoing a course of treatment with a particular drug, and the method may comprise treating the subject with the same drug (e.g. carrying on the course of treatment) when the subject's condition is determined to have improved or remained stable. Alternatively, the method may comprise treating the subject with a different drug when the subject's condition is determined to have worsened.

An autoimmune disease may be selected from lupus nephritis, neonatal heart block, primary Sjögren syndrome, CREST syndrome, scleroderma, systemic lupus erythematosus (SLE), inflammatory myopathy, mixed connective tissue disease, systemic sclerosis, drug-induced lupus erythematosus, primary biliary cirrhosis, autoimmune-mediated heart disease, autoimmune-mediated cardiomyopathy, Raynaud's syndrome, psoriasis, multiple sclerosis, chronic fatigue syndrome/myalgic encephalomyelitis, celiac (or coeliac) disease, dermatitis herpetiformis, Miller Fisher syndrome, acute motor axonal neuropathy (AMAN), multifocal motor neuropathy with conduction block (MMN), autoimmune hepatitis, gastric cancer, rheumatoid arthritis, antiphospholipid syndrome, granulomatosis with polyangiitis, microscopic polyangiitis, eosinophilic granulomatosis with polyangiitis, systemic vasculitides, chronic autoimmune hepatitis, dermatomyositis, scleromyositis, myasthenia gravis, Lambert-Eaton myasthenic syndrome, small intestinal bacterial overgrowth, Hashimoto's thyroiditis, Graves' disease, paraneoplastic cerebellar degeneration, limbic encephalitis, encephalomyelitis, subacute sensory neuronopathy, choreathetosis, paraneoplastic cerebellar degeneration, opsoclonus myoclonus syndrome, stiff person syndrome, diabetes mellitus type 1, Isaac's Syndrome (autoimmune neuromyotonia), optic neuropathy, chorea, Sydenham's chorea, paediatric autoimmune neuropsychiatric disease associated with Streptococcus (PANDAS), anti-NMDA receptor encephalitis, neuromyelitis optica (Devic's syndrome), Pemphigus vulgaris, Bullous pemphigoid, Goodpasture syndrome, Pernicious anemia, Membranous nephropathy, acquired neuromyotonia, cerebellar ataxia, progressive encephalomyelitis with rigidity and myoclonus, demyelinating inflammatory disorders, neuromyelitis optica, acute disseminated encephalomyelitis (ADEM), autoimmune thyroiditis, Morvan's syndrome, arthrogryposis (i.e. fixed joint contractures), autism, schizophrenia, epilepsy, dementia or psychosis.

The autoimmune disease may cause increased levels of circulating autoantibodies of a particular class. For example, a patient with an autoimmune disease may have increased levels of circulating igM and/or igG antibodies due to the production of igM and/or igG autoantibodies. Thus, the method may comprise determining the fraction of igG B cells (or class-switched B cells including igG B cells_ in a sample from the patient, such as e.g. a blood sample. For example, individuals with systemic lupus erythematosus and rheumatoid arthritis are characterized by high levels of circulating igM and igG antibodies. The autoimmune disease may be an autoimmune disease or condition resulting from the presence of autoantibodies during development. In some embodiments, the autoimmune disease may affect a developing foetus. In some embodiments, the autoimmune disease affecting the developing foetus may be caused by maternal autoantibodies, produced by the mother. Thus, the method may comprise determining the fraction of igG B cells (or class-switched B cells including igG B cells) in a sample from a pregnant mother, such as e.g. a blood sample, amniotic fluid sample, chorionic sample, etc.

The methods of the disclosure may also be used to monitor, diagnose, and/or prognose transplant rejection, in patients that receive an organ transplant. The organ transplant may be an allogenic organ transplant. The transplanted organ may or may not be HLA matched to the recipient. The organ transplant may cause the production of anti-HLA antibodies. The anti-HLA antibodies may be igG antibodies. The antibodies produced may be alloantibodies, i.e., produced in response to host antigens. Alternatively, the antibodies may be autoantibodies, i.e., pre-existing antibodies with specificity for host antigens. The antibody level may indicate the risk of transplant rejection in a recipient patient, for example, increased level of igG antibodies may indicate an increased risk of rejection. Thus, the method may comprise determining the fraction of igG B cells (or class-switched B cells including igG B cells) in a sample from a subject who has received an organ transplant, such as e.g. a blood sample, a sample from the grafted organ or adjacent tissue. An elevated level of such cells in the sample, compared to a control (such as e.g. a control sample or cohort, or a level previously determined for the same subject) may indicate a high risk of transplant rejection. The organ transplant may be a solid organ transplant selected from: kidney, lung, liver, heart, and pancreas. The organ transplant may be a bone marrow, stem cell, or T cell transplantation, or a blood transfusion. The organ transplant may be a tissue transplant, selected from: skin, bone, tendon, ligament, heart valve, blood vessels, and cornea. The method may further comprise treating a subject who has been identified as being at high risk of transplant rejection, for example by administering an immunosuppressant.

### Systems

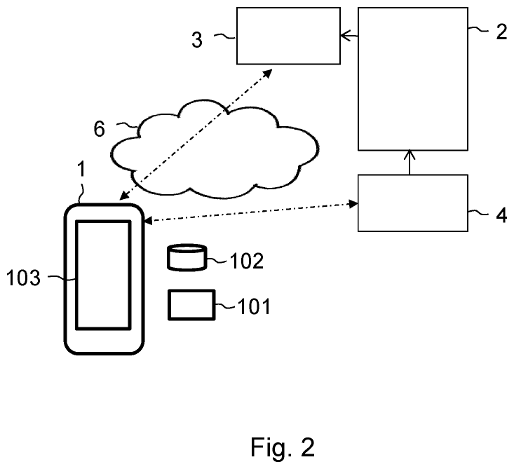

FIG. 2 shows an embodiment of a system for determining the B lymphocyte fraction in a sample and/or for providing a prognosis or treatment recommendation based at least in part on the lymphocyte fraction, according to the present disclosure. The system comprises a computing device 1, which comprises a processor 101 and computer readable memory 102. In the embodiment shown, the computing device 1 also comprises a user interface 103, which is illustrated as a screen but may include any other means of conveying information to a user such as e.g. through audible or visual signals. The computing device 1 is communicably connected, such as e.g. through a network 6, to read depth data acquisition means 3, such as a sequencing machine, and/or to one or more databases 2 storing read depth data. The one or more databases may additionally store other types of information that may be used by the computing device 1, such as e.g. reference sequences, parameters, etc. The computing device may be a smartphone, tablet, personal computer or other computing device. The computing device is configured to implement a method for determining the lymphocyte fraction in a mixed sample, as described herein. In alternative embodiments, the computing device 1 is configured to communicate with a remote computing device (not shown), which is itself configured to implement a method of determining the B lymphocyte fraction in a sample, as described herein. In such cases, the remote computing device may also be configured to send the result of the method of determining the B lymphocyte fraction to the computing device. Communication between the computing device 1 and the remote computing device may be through a wired or wireless connection, and may occur over a local or public network such as e.g. over the public internet or over WiFi. The read depth data acquisition means may be in wired connection with the computing device 1, or may be able to communicate through a wireless connection, such as e.g. through WiFi, as illustrated. The connection between the computing device 1 and the read depth data acquisition means 3 may be direct or indirect (such as e.g. through a remote computer). The read depth data acquisition means 3 are configured to acquire read depth data from nucleic acid samples, for example genomic DNA samples extracted from cells and/or tissue samples. In some embodiments, the sample may have been subject to one or more preprocessing steps such as DNA purification, fragmentation, library preparation, target sequence capture (such as e.g. exon capture and/or panel sequence capture). Any sample preparation process that is suitable for use in the determination of a genomic copy number profile (whether whole genome or sequence specific) may be used within the context of the present disclosure. The read depth data acquisition means is preferably a next generation sequencer. The sequence data acquisition means 3 may be in direct or indirect connection with one or more databases 2, on which sequence data (raw or partially processed) may be stored.

The following is presented by way of example and is not to be construed as a limitation to the scope of the claims.

### EXAMPLES

### Example 1: a Method for Calculation of B Cell Fraction from WES or WGS Data

This example describes a method for determining the (total) B cell fraction in a mixed sample using a signal from the IGH locus on human chromosome 14, from WES or WGS data.

B cell diversity is a product of VDJ recombination, where the different gene segments within the B cell receptor gene recombine. The result of this is the excision of a large number of unselected gene segments from the IGH gene as TRECs. This process leads to a copy number difference between B cells and other cells, with the B cells effectively undergoing a deletion event within the IGH gene.

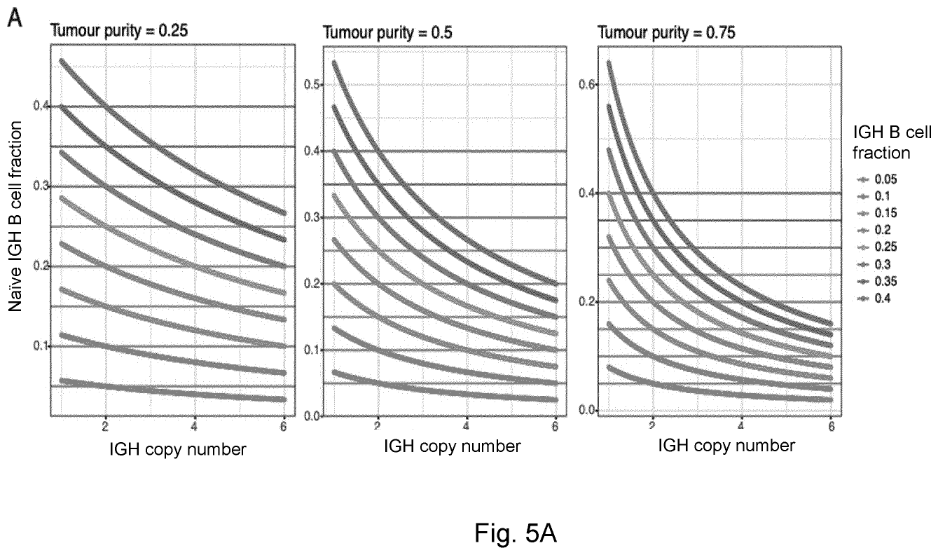

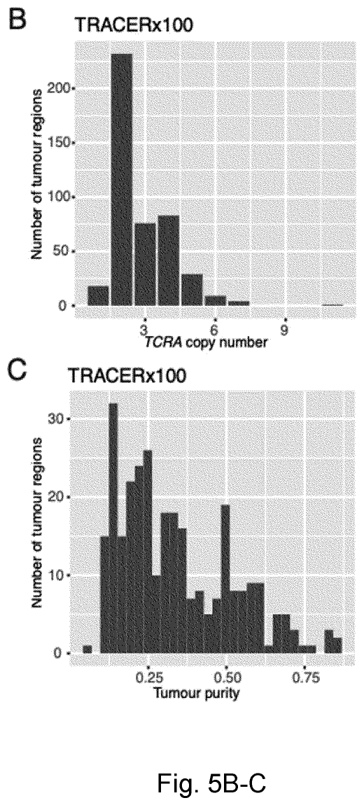

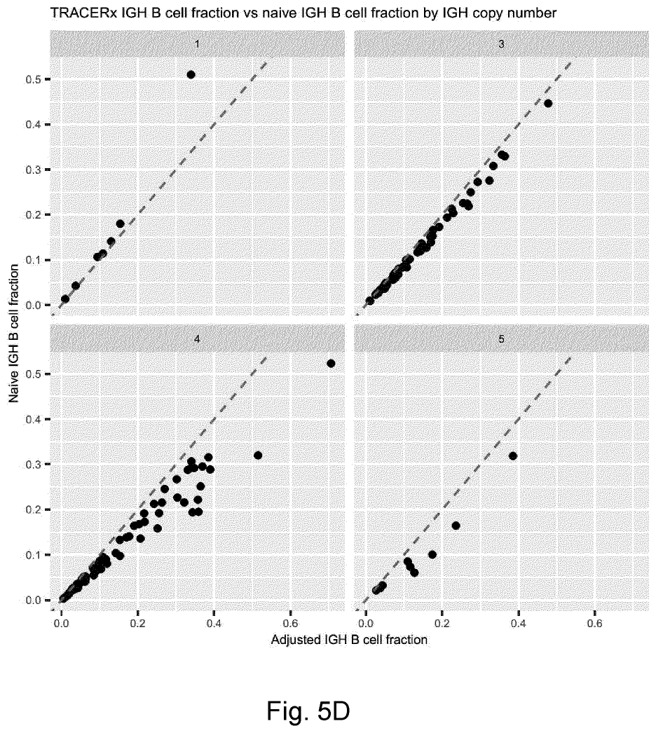

To explicitly exploit this signal to calculate a B cell fraction within individual samples, the inventors developed a method that relies on an analysis of the read depth ratio within the IGH gene to directly measure B cell fraction in WES or WGS samples. First, the read depth at each position within the IGH gene is calculated, using the genomic segments at the beginning and end of the IGH locus that are unaffected by VDJ recombination or class-switch recombination (CSR) as a control, and a modified log read depth ratio is calculated between the original read depth of the coverage values and the median of the values within the control genomic segments. Finally, provided both the fraction of tumour cells within the sequenced sample (the tumour purity) and the local somatic copy number around IGH is known, an exact estimation of the B cell fraction can be calculated based on the size of the deviation of the log (base 2) read depth ratio in regions subject to VDJ recombination (for the total B cell fraction) and regions subject to CSR (for the class-specific B cell fraction). If knowledge of the tumour purity and local somatic copy number is unavailable (or the sample is not expected to comprise cells with aberrant copy numbers, i.e. all cells are expected to have normal ploidy in the region of the IGH locus), a naïve estimation can also be made which the inventors demonstrate to have a similar predictive value (as explained in detail in the methods section and as illustrated on FIG. 5).

Notably, unlike RNA-seq scores, the IGH score represents a direct measurement of the proportion of B cells (or B cells within a particular class). This is referred to from here as the “B cell fraction” (or “total B cell fraction” when referring to all B cells indistinctive of class, calculated from regions of the IGH gene undergoing VDJ recombination, and “class-specific B cell fraction”, “class-switched B cell fraction”, “non-class-switched B cell fraction” (also referred to as “naïve B cell fraction” or “igM/D B cell fraction”), “igG B cell fraction”, “igA B cell fraction”, or “igE B cell fraction” when referring to fractions of B cells in one or more specific B cell classes).

A full description of the method along with the optimisation of its parameters such as the choice of segments for normalisation and quality control is presented in the Methods below.

In particular, the methods below describe the general concept behind the methods described herein, its implementation in the context of WES data, and its extension to WGS data. Unlike whole exome sequencing (WES), WGS contains coverage across the entire genome. In the context of estimating copy number alterations, this provides a number of important advances, 1) uniform coverage allows for the accurate identification of the location of copy number breakpoints and 2) there are fewer biases such as those due to either GC content or reference allele bias. The methods described herein when applied to WGS may enable to estimate T or B cell fraction to a greater accuracy at a lower depth. This may be particularly beneficial as many WGS data sets lack accompanying immune data. Therefore, the methods described herein can provide a comprehensive immune profile of a sample of any WGS sample without the need for any additional data. Further, current WES data was found to be particularly noisy at the IGH locus, and the use of WGS data was therefore found to be particularly beneficial in the context of determining B cell fractions. A segment-based model that takes particular advantage of the additional resolution in WGS data was developed, with the ability to quantify V(D)J gene segment usage in the TCR and IGH loci.

By adapting this segment model, the inventors were able to design a new method to quantify class switched B cell fractions within the total B cell fraction, by analysing regions of the IGH locus that undergo CSR.

### Definition of Local Copy Number Values Around the IGH Locus in Different Cells within a Tumour Sample

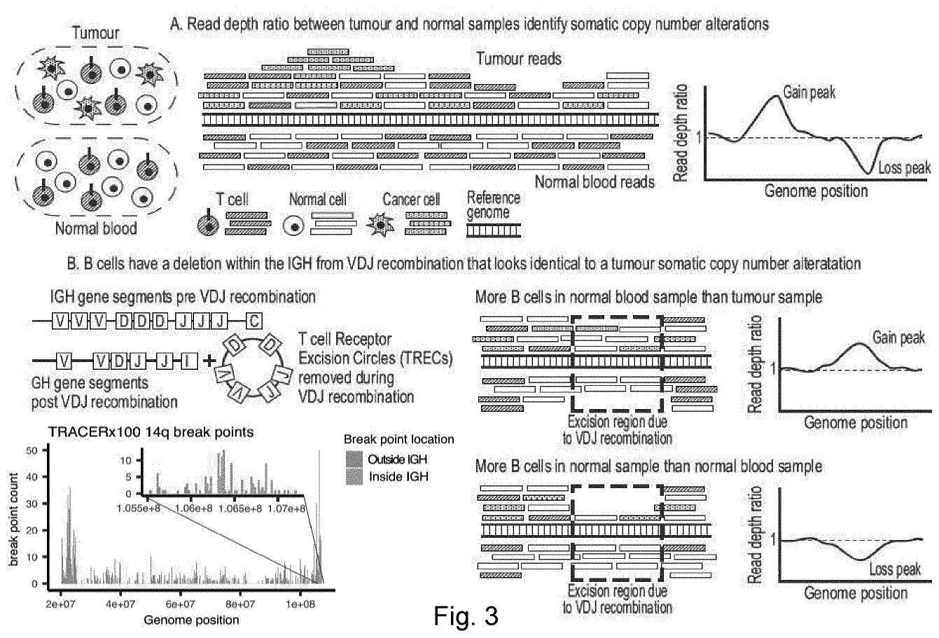

Within B cells and other stromal cells which are known to be diploid there are two copies of the IGH locus and the copy number around the IGH locus can be said to equal 2. Assuming that within B cells there is a region within the IGH locus that is always lost during VDJ recombination, this leads to a copy number of 0 in this small genomic segment (as illustrated on FIG. 3). In tumour cells there can be both copy number gains and losses across chromosome 14 (where the IGH locus is located) leading to different possible copy number states of the IGH locus that may differ from 2. The present method assumes that there are no break points within the IGH locus originating from somatic alterations in cancer cells (although the region as a whole may not be diploid in tumour cells).

As explained in more detail in the two subsections below, the mean copy number at the start of the IGH locus (chr14:106032614 (Hg19), corresponding to chr14:105566277 (Hg38)) is inferred from all cancer cells in the sample using ASCAT (Van Loo et al., 2010), and is referred to as the local tumour copy number at the IGH locus. This is used as a term in the SCNA-aware IGH B cell fraction provided by equation (7) below. Additionally, when tumour somatic copy number alteration information is not available, a SCNA-naive IGH B cell fraction (equation (3)) can be used, and is referred to as the naive IGH B cell fraction.

### Calculation of IGH B Cell Fractions

The ASCAT approach (allele-specific copy number analysis of tumours) described in Van Loo et al. (2010) provides a method for estimating allele-specific copy numbers from DNA extracted from samples that include a mixture of tumour cells and non-aberrant cells. The formula below is the equation used by ASCAT to estimate tumour ploidy at a genomic location i, in combination with a second equation for the B-allele frequency at the location (see Van Loo et al.):

$${r}_{ι} = γ {log}_{2} ( \frac{2 ( 1 - p ) + p ( {n}_{Ai} + {n}_{Bi} )}{{Ψ}_{S}} ) \qquad ( 1 )$$

where rᵢ is the log R (a measure of total signal intensity, quantifying the total copy number at a genomic locus) at a location i, γ is a constant dependent on the technology used (for example, a value of γ=1 may be used for Illumina Hiseq), p is the tumour purity (fraction of tumour cells amongst the population of cells in the sample), n_{Ai} and n_{Bi} are the allele specific copy number values, the factor 2 represents the diploid copy number assumed for normal tissue, and ψₛ is the average ploidy of the tumour sample. When using ASCAT with sequencing data, rᵢ is the log (base 2) of the read depth ratio at genomic location i, where the ratio is between the coverage for a tumour sample and a germline sample at the same genomic location.

In the present work, the aim was to identify B cell fractions in a sample amongst a population of cells in a sample, and in particular the total B cell fraction and/or the fraction of B cells of a particular class, including igG, igA, igE, and naive non-class switched B cells igM/D, using sequence data from the IGH locus. Further, the sample may contain aberrant cells (e.g. tumour cells) which may have unknown ploidy at the genomic locus I (in this case the IGH locus). By analogy with the mixed population investigated in Van Loo et al., the rᵢ value would represent the ratio of coverage between a sample comprising B cells amongst other (non-B) cells, and a sample without B cells. For example, tumour biopsy samples and matched blood samples are frequently available for tumour patients. In practice, contrary to the tumour situation where a non-tumour sample can be used to quantify rᵢ, no sample from a patient is known to definitely lack any B cells. Indeed, both a tumour sample and a matched blood sample are likely to contain B cells. Thus, a different formula is needed in this case for estimating the log R from a single sample. Briefly, this is calculated by using the median coverage of genomic regions at normalisation regions, chosen at the extreme beginning and end of the IGH locus, as a normal background rate that is assumed to have copy number values unaffected by any VDJ or class switch recombination (Hg38: chr14:105566277-105588395 and chr14: 106779812-106879844, corresponding to Hg19: chr14: 106779812-106879844 and Hg19: chr14: 107188051-107288051). Then, by dividing the coverage across the IGH genomic region by this median value the inventors create an estimate for rᵢ across the entire IGH locus (Hg19: chr14: 106032614-107288051, corresponding to Hg38: 105566277-106879844). Thus, the present case can be expressed as:

$${r}_{i} = γ {log}_{2} ( \frac{{Ψ}_{Si} ( 1 - f ) + f ( {n}_{T} )}{{Ψ}_{S}} ) \qquad ( 2 )$$

where f is the B cell fraction of the sample (which can be either the total B cell fraction (f^{tot}), when looking at the a locus in the IGH gene that is lost in substantially all B cells by VDJ recombination, or the fraction of B cells that comprise a particular class switch segment (f^{class}) when looking at a locus in the IGH gene that is lost in substantially all B cells in that particular class by CSR; the notation f encompasses both f^{class} and f^{tot} unless context indicates otherwise), n_{T} is the copy number of the B cell at the respective location in the IGH locus, γ is a constant dependent on the technology used (for example, a value of γ=1 may be used for Illumina Hiseq), rᵢ is the log R calculated from a single sample as explained below, ψₛᵢ is the copy number for the sample excluding B cells at the IGH locus (expected to be constant across the IGH locus), and ψₛ is the copy number at this locus for the entire sample without taking into account any changes due to VDJ or class switch recombination. Assuming that we are looking at the maximum point of VDJ or class switch recombination (where r_{B} takes values referred to as r_{VDJ} and r_{CSR}, respectively), it can be assumed that n_{T}≈0 (i.e. the copy number of the B cells or the particular B cell class under investigation is 0). Thus, at this location, equation (2) simplifies to equation (2b), wherein f may be f^{tot} or f^{class} and r_{B} may be r_{VDJ} or r_{CSR}, respectively:

$${r}_{B} = γ {log}_{2} ( \frac{{Ψ}_{Si} ( 1 - f ) )}{{Ψ}_{S}} ) \qquad ( 2 b )$$

$$or$$

$$f = ( {Ψ}_{S i} - {Ψ}_{S} {2}^{\frac{{r}_{B}}{γ}} ) / {Ψ}_{S i} \qquad ( 2 c )$$

At this point, two different situations may be present:
(a) it can be assumed that Ψ_{S}≈Ψ_{Si}—for example in normal (i.e. non-tumour) samples, where the average ploidy will always be 2 (e.g. in the case of a blood sample), or where the average ploidy may not be 2 but the fraction of B cells present is small (such as e.g. at most 10%, or at most 5%, e.g. cancer samples with low B cell content, or igE class B cells which only make up a small proportion of the total B cell fraction);
(b) the above assumption does not hold, for example in tumour samples where the B cell content is not small.

Case (a) lends itself to a straightforward simplification of equation 2, into equation (3), wherein f may be f^{tot} or f^{class} and r_{B} may be r_{VDJ} or r_{CSR}, respectively:

$$f = 1 - {2}^{\frac{{r}_{B}}{γ}} \qquad ( 3 )$$

This is referred to as a “naive IGH B cell fraction” equation.

For case (b), the following equations for Ψ_{S} and Ψ_{Si} can be used:

$${Ψ}_{Si} = 2 ( 1 - {p}^{′} ) + {p}^{′} {Ψ}_{T} \qquad ( 4 )$$

$${Ψ}_{S} = 2 ( 1 - p ) + p {Ψ}_{T} \qquad ( 5 )$$

$${p}^{′} = \frac{p}{1 - f} \qquad ( 6 )$$

where ψ_{T} is the local tumour copy number at the IGH locus (calculated as the mean copy number at the start of the IGH locus in these examples, i.e. Hg19: chr14: 106032614—although any region of the IGH locus that does not undergo VDJ or class switch recombination could be used such as e.g. the normalisation regions mentioned above, inferred from all cancer cells in the sample using ASCAT), and p′ is the adjusted tumour purity taking into account the absence of any B cell DNA at the location of maximum VDJ recombination (when f is f^{tot}) or the location of maximum CSR recombination (when f is f^{class} and the class under investigation is all class-switched B cells) or the f calculated for a population including a particular segment (when f is f^{class} and the class under investigation is a particular class of B cells). In these examples, ψ_{T} is calculated as the mean copy number from all cancer cells (i.e. across all subclones, where respective subclones may have different copy numbers at the locus) at start of the IGH locus because this region is not expected to show signal from VDJ recombination. Thus, the end of the gene (i.e. any part of the IGH gene that is not expected to show VDJ recombination or CSR) could be used instead of the beginning in any embodiment, without affecting the results, as this also is not expected to show any signal from VDJ or class switch recombination. Without wishing to be bound by theory, it is believed that the use of the signal at the beginning of the gene is beneficial because the signal associated with VDJ recombination is closer to the end of the gene than to its beginning (due to the J segments being clustered close to the end of the gene, whereas the V segments are more spread out), as is the signal associated with CSR recombination (which is located in the constant region following the VDJ segments). Additionally, it would also be possible in any embodiment to use the mean copy number from all cancer cells at a region outside of the locus under investigation (if such data is available, for example when whole genome sequencing is used rather than whole exome sequencing), such as e.g. before or after the locus. However, increasing distances from the locus of interest increase the likelihood that the signal seen will correspond to a different copy number event in the cancer cells, and hence no longer reflect the tumour copy number at the locus under investigation.

Substituting equations (4), (5) and (6) into equation (2) produces equation (7):

$$f = ( 1 - p + \frac{p}{2} {Ψ}_{T} ) - ( 1 - p + \frac{p}{2} {Ψ}_{T} ) {2}^{\frac{{r}_{B}}{γ}} \qquad ( 7 )$$

Equation (7) represents a “more complete” version of equation (3) (i.e. a solution of equation (2) that does not make all of the assumptions made to arrive at equation (3). Thus, by applying the same assumptions starting from equation (7), it is possible to recover equation (3). In particular, in cases where these are no cancer cells (no cells with a ploidy that differs from the normal 2; p=0), equation (7) immediately simplifies to equation (3). Similarly, where the local copy number around IGH in any tumour cells present is the same as in normal cells (ψ_{T}=2) this formula also simplifies to that in equation (3). Thus, Equation (3) can be used to calculate the total IGH B cell fraction (when the maximum point of VDJ recombination in the IGH locus is used) or the fraction of a particular B cell class (when the read depth ratio in a particular region of the IGH locus corresponding to a segment lost in the class is used) in samples from normal tissue or as a naive estimate where the tumour purity or copy number status is unknown.

In other words, the non-cancer component (1−p) of a sample can be rewritten by dividing it into a B cell and non-B cell subset:

(1−p)=(1−p)f′+(1−p)(1−f′)

where f′ represents the fraction of non-tumour cells which are B cells and is related to the total B cell fraction of the sample that are B cells (f) as follows:

$${f}^{′} = \frac{f}{( 1 - p )} .$$

Letting the total copy number of the tumour at genomic loci i equal to ψ_{T} ignoring allele specific copy number, and the copy number of the B cell compartment equal to n_{T} and the copy number of the non-B cell normal cell compartment to be equal to 2 at all genomic loci leads to the following equation:

$${r}_{i} = γ {log}_{2} ( \frac{{n}_{T} ( 1 - p ) {f}^{′} + 2 ( 1 - p ) ( 1 - {f}^{′} ) + p {Ψ}_{T}}{{Ψ}_{S}} )$$

Now consider the genomic location at the VDJ recombination/CSR event at the IGH locus in B cells, assuming at this location that n_{T}→0, additionally at this location r_{B} is directly estimated from the read depth ratio as described herein. ψ_{S} is calculated as the average copy number of the sample without taking into account VDJ recombination/CSR: ψ_{S}=2(1−p)+pψ_{T}. Therefore, at the genomic loci of VDJ recombination/CSR we have the following equation:

$${r}_{B} = γ {log}_{2} ( \frac{2 ( 1 - p ) ( 1 - {f}^{′} ) + p {Ψ}_{T}}{2 ( 1 - p ) + p {Ψ}_{T}} ) .$$

Substituting the equation for f′ into this equation and rearranging the equation for the B cell fraction within a sample can be found to be provided by equation (7) above. Note that if the value of the log ratio r_{B} is >0 then the resulting fraction will be negative. In these cases, it may be assumed that there are no B cells present in the sample and only sample noise is being measured. Thus, f may be set to 0. Conversely if the tumour purity p is known (and/or it can be assumed that there is no error in the calculation of the tumour purity), then any values of f higher than 1−p are not plausible (since the tumour purity+B cell fraction of a sample cannot be greater than 1) and f can be rounded down to this value if a higher value is obtained.

To calculate the B cell fraction from equation (3) (for a naïve estimate of B cell content) or equation (7) (if tumour copy number and purity are known), the log read depth ratio rᵢ needs to be estimated from the raw coverage data (i.e. from a single sample, rather than from matched samples comprising and not comprising, respectively, a population of cells to be quantified).

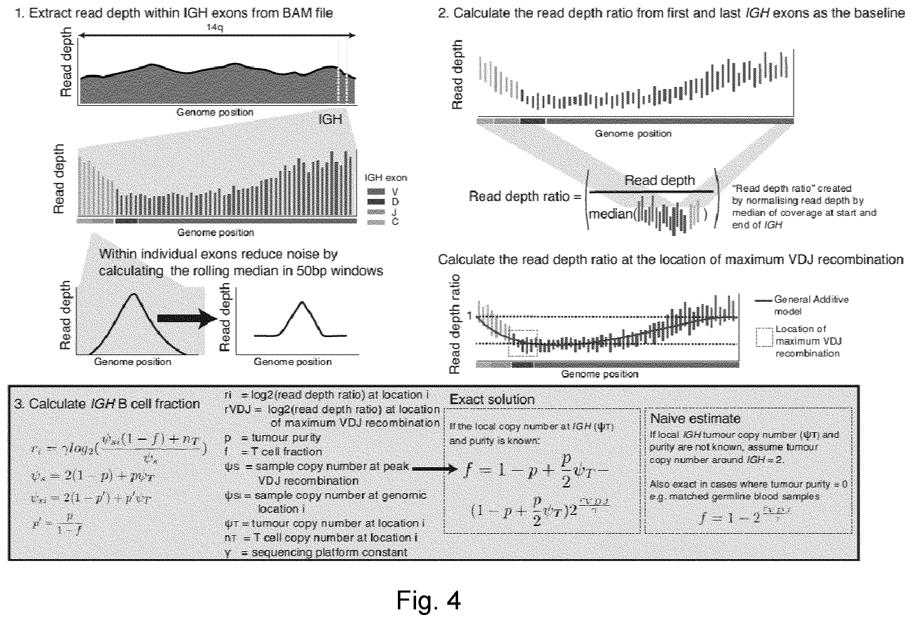

In short, this log R is calculated by using the median coverage of genomic regions at the extreme beginning and end of the IGH locus as a normal background rate that is assumed to have copy number values unaffected by any VDJ or class switch recombination. As explained above, it would also be possible in any embodiments to use the beginning or end of the VDJ locus under investigation (instead of both), or other (preferably nearby) regions that can be assumed to have copy number values unaffected by VDJ or class switch recombination. For example, other regions within the constant region of the IGH locus may be used, providing they do not overlap with the gene segments that undergo class switch recombination. Without wishing to be bound by theory, it is believed that the use of longer normalisation regions (e.g. using both the beginning and end regions, and/or increasing lengths of the beginning region) advantageously compensates for noise that is typical in read depth data. For example, the beginning region can be extended to include a number of V segments that are unlikely to be frequently lost, or even regions outside of the gene if this data is available (such as e.g. when using whole genome sequencing data). In the present case, the following regions were used: chr14: 106779812-106879844 and chr14: 107188051-107288051 in hg19, and chr14:105566277-105588395 and chr14: 106779812-106879844 in hg38. Then, dividing the coverage across the IGH genomic region by this median value results in an estimate for rᵢ across the entire IGH locus (in this case, chr14: 106032614-107288051 in hg19, corresponding to chr14: 105566277-106879844 in hg38). The idea behind this is illustrated in FIG. 4.

A model is then fitted to this data. Different models were developed for WES and WGS data, as explained below. The average value of the model at the location of maximum VDJ recombination (r_{VDJ}—when looking in the regions of the IGH locus that undergo VDJ recombination) or the average change in the value of the model between a CSR segment and preceding segment (r_{CSR}—when looking in the regions of the IGH locus that undergo CSR) is taken as the value for r_{B}. Equation (7) or equation (3) is then used to derive a fraction f, which is the total B cell fraction (when r_{B} is the value of the model at the location of maximum VDJ recombination), or the fraction of B cells that make use of a particular class switch segment (when r_{B} is the change in the value of the model between the class switch segment being considered and the preceding segment).

As explained above, for many tumour samples, equation (7) can be used, requiring knowledge of both tumour purity and the tumour copy number at the IGH locus. In the special case when the copy number at IGH is exactly 2, as is true in all blood derived samples and many tumours, equation (3) can be used to calculate the B cell fraction, and is exact. Additionally, if the tumour purity and tumour copy number at IGH are unknown, equation (3) can function as a naïve estimate for B cell fraction (although depending on whether the above mentioned assumptions hold true or not, this naïve estimate may not be exact).

Calculation of Log Read Depth Ratio from a Single Sample in WES

In more detail, from an aligned BAM file to either hg19 (based on GRCh37) or hg38 (GRCh28) (depending on the data set, e.g. the TCGA data was obtained pre-aligned to hg38, whereas the TRACERx and other data was aligned in house to hg19) the coverage at an individual base level is extracted using the samtools (version 1.3.1) depth function (with parameters -q 20 -Q 20). Once this is done, only bases within known exons as defined by the SureSelect human all exons probes (version 5) used within TRACERx, within each exon the reads are then normalised with a rolling median in windows of 50 base pairs (i.e. the median value within each 50 bp window is taken to be the new value). Note that the size of window is not fixed and in embodiments windows of fewer than or more than 50 bp can be used, such as e.g. between 20 and 200 bp, or between 50 and 150 bp. Suitable sizes of windows can be determined empirically for a particular data set, type of data or context, by comparing the lymphocyte fraction estimates obtained using the present method with various candidate window sizes to orthogonal metrics of lymphocyte fractions such as the Danaher score.

The median baseline coverage value is calculated in regions of IGH assumed not to be affected by VDJ recombination, taking regions at the beginning and end of the gene. All coverage values are then divided by this value, with the log then being taken to calculate a ‘single sample’ log ratio.

To complete the calculation of r_{B} a general additive model is fitted to the data with the average value of the model at the location of maximum VDJ recombination (when looking in the regions of the IGH locus that undergo VDJ recombination) or CSR (when looking in the regions of the IGH locus that undergo CSR) being taken as the value for r_{B}. A generalized additive model is a generalized linear model in which the linear predictor is given by a sum of smooth functions of the covariates plus a conventional parametric component of the linear predictor. The model effectively fits a smoothed line to the data, and any approach to do this may be used in the context of the disclosure (e.g. using any types of generalised linear model or any other smoothing approach such as e.g. fitting a piecewise constant model). In this case, the model was fitted using the “geom_smooth” function in R, which uses the “gam” function from the mgcv package (mgcv::gam) with formula y~s(x, bs=“cs”) (see https://rdrr.io/cran/mgcv/man/gam.html for details). This implementation represents the smooth functions of the generalised additive model using penalised regression splines. A diagram of this process is shown on FIG. 4. The location of maximum VDJ recombination was defined as the gap between the final IGH J segment and the first IGH V segment, i.e. region chr14:105865679-105939755 in Hg38—as explained below. Other definitions are possible and small changes in the size of these regions (in particular the region of maximum VDJ recombination) are not expected to make much difference. In general, a regions of maximum VDJ recombination may be defined for a VDJ locus by defining a region located at least partially around or within the region between the final J gene segment and the first V gene segment, such as a region corresponding to the gap between the final J gene segment and the first V gene segment, optionally rounded for ease of manipulation, such as e.g. to the nearest thousand bp or the nearest 10k bp.

For the identification of classes of B cells, each class fraction was obtained using fractions calculated from segment usage for one or more CSR segments, using a segment-based model as described further below. The locations of segments used here are provided in Table 2 below. However, it is possible to calculate at least the fraction of class switched B cells from WES data, using a fitted GAM model (or other model that can be fitted to WES data). In particular, a general additive model can be fitted to the data with the average value of the model at the location of maximum CSR being taken as the value for r_{B}, as explained above. It is preferable for separate models to be fitted to the VDJ region and the CSR region, in order to ensure that the fitted values correctly capture the present of two separate deletion events. The location of maximum CSR may be defined as a region between the IGHG3 segment and the first J segment, i.e. region chr14:[105771405-105863198] in Hg38—as explained below. Other definitions are possible and small changes in the size of these regions (in particular the region of maximum CSR) are not expected to make much difference. In general, a regions of maximum CSR may be defined as a region located at least partially around or within the region between the first J gene segment and the last class switching segment (IGHG3), such as a region corresponding to the gap between the IGHG3 segment and the first J gene segment, optionally rounded for ease of manipulation, such as e.g. to the nearest thousand bp or the nearest 10k bp.

Calculation of Log Read Depth Ratio from a Single Sample in WGS

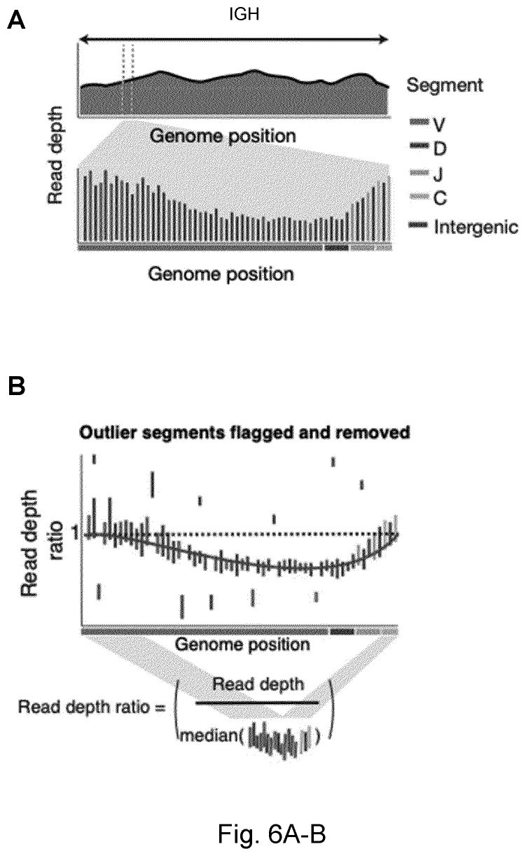

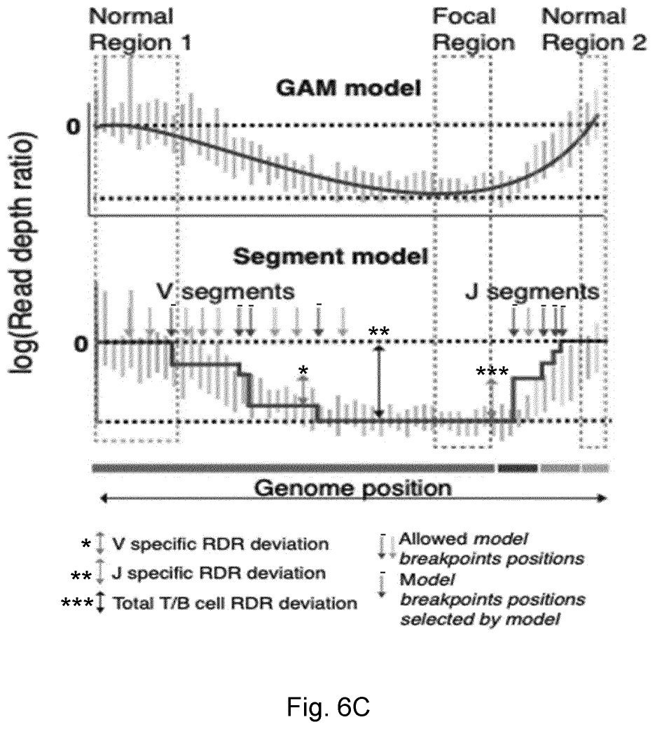

The method described above can be tailored to WGS by applying the GC correction and quality control to 1000 bp windows instead of exon segments from WES capture kits and applying additional quality control to identify any 100 bp genomic window that had evidence of being an outlier in terms of having a much higher or lower read depth than nearby regions (see methods for full details). An overview of this adapted method (referred to as “adapted GAM”) is given in FIGS. 6A-B, FIG. 6C top panel.

Alternatively, a segment-based model can be used as will now be explained (illustrated on FIG. 6C, bottom panel). The method described above for WES data uses a GAM model to calculate r_{B} and hence the B cell fraction within a sample. The GAM model simplifies modelling the process of recombination as a smoothed process. This is useful when using WES data due to the non-uniformity of coverage with large segments of the genome within the IGH locus not being sequenced by any exome capture kit. This comes at a cost of increased vulnerability to noise within the data. Further, this is not constrained to match the known biological reality of the process of V(D)J/CS recombination. The benefits outweigh the costs in the case of WES data when the coverage within the locus under investigation is not uniform. However, using WGS data (or any other data not suffering from this bias due to the particular regions captured) it is possible to use a model that is based directly on the biological process of V(D)J/CS recombination. This was implemented by modelling the read depth ratio as a series of constant piecewise segments, with the location of breakpoints representing possible beginnings of deletion sites following V(D)J/CS recombination, which are known from the location of the V and J gene segments and IG segments, respectively, and thus can be provided as constraints to the model. Thus, the inventors created a model where the segments are pre-chosen and align with the locations of the V and J segments (for determining the total B cell fraction), or the locations of the IG segments (IGHM, IGHD, IGHG3, IGHG1, IGHA1, IGHG2, IGHG4, IGHE and IGHA2, as will be explained in more detail in Example 2). Additionally, the model can be constrained by the knowledge from V(D)J recombination that the read depth ratio should start from 0 and from there must be monotonically decreasing until the point of maximum V(D)J recombination. For example, in IGH V(D)J recombination only some BCR chains will have selected the first IGHV-1 segment and all other V segments will be deleted, however all BCR chains will have a deletion following the final V segment. Likewise for J segments these must be monotonically increasing until reaching 0.

To fit this model, each possible segment with known break points corresponding to the V and J genes (see Table 1) is transformed into vectors of 1s and 0s, which are equal to 1 within their region and 0 outside. These vectors are then fit to the normalised read ratio data using a constrained linear model (using functions from the R package restriktor v0.3), with inequality constraints chosen as follows for each of the n V segments and m J segments: V₁<0; V₂<V₁; . . . ; V<Vₙ₋₁; J₁>V₁; J₂>J₁; . . . ; Jₘ>Jₘ₋₁; Jₘ<0. Using these fitted values the total B cell fraction can be calculated from the maximum deviation of the model e.g. the value from the last V segment as well as individual fractions from individual segments. A similar model with adapted constraints is fitted to determine the class specific B cell fractions as will be explained in more detail in Example 2.

### Bayesian Model

As an alternative to the generalised additive model and the piecewise constant model (segment model) described above, a Bayesian model may be used to model the read depth ratio profile. For example, the model may be a Bayesian modelling segment usage from the read depth and a prior distribution of usage of each of a plurality of segments (e.g. V and J genes or CSR segments) in the region of interest. A prior distribution of usage of each of a plurality of segments (V and J genes or CSR segments) in the region of interest may be a uniform distribution. Such a distribution may define an equal likelihood for each segment to be used. A prior distribution of usage of each of a plurality of segments in the region of interest may be a distribution using prior knowledge of segment usage in a reference population. For example, B cell receptor sequencing data obtained from a reference population could be used to determine the distribution of V and J gene usage and/or class switch segments in the reference population. The reference population may be a human population. The reference population may be a human population with one or more specific characteristics selected from age, sex and HLA type. For example, the reference population may be selected to have characteristics that match a sample that is analysed. This approach may be particular suited to WGS data. A Bayesian model may be implemented using a gradient-based Markov chain Monte Carlo (MCMC) method to calculate the probability distributions for the segment usage of each segment (e.g. V and J gene or CSR segment) in the region of interest.

### Naïve BCRA T Cell Fraction Vs Exact Estimate of B Cell Fraction

FIG. 5A shows the (theoretical) difference between the estimated naïve value for B cell against different real B cell fraction values for a range of local copy number values and tumour purities based on theoretical values as derived from equations (2), (3) and (7). At low B cell fractions and local copy number values close to 2 this estimate is very accurate. FIGS. 5B-C shows using real data from the TRACERx100 cohort (more information on this data is provided in Example 2) that the distribution of local TCRA copy number has a mode of 2, while tumour purity values are typically low. The same was true for the IGH locus (data not shown). For cases where the local IGH copy number is not 2, FIG. 5D shows the correlation between the naïve estimate and exact calculated B cell fraction.

### Selection of Segments Used for Estimation of the Log Ratio (r_{B})

The calculation of r_{B} requires a choice of (i) a segment of maximum VDJ/segments of class switch recombination, and (ii) one or more segments to be used to calculate the “normal baseline” to which the coverage across the IGH genomic region is compared (by calculating a ratio, as explained above).

For the focal segment representing the position of maximum VDJ recombination, a gap between the final V gene segment and first segment that encodes part of the constant region was used (chr14: 105865679-105939755 in Hg38). As explained above, any region between the final V segment and the first J segment of the VDJ locus of interest may be used.

For the segments representing the position of CSR in each of a plurality of B cell classes, segments in the constant portion of the IGH gene corresponding to one or more of the IGHA2, IGHE, IGHG4, IGHG2, IGHA1, IGHG1, IGHG3, IGHD and IGHM regions were used. Exemplary coordinates for these are shown in Table 2. These coordinates were obtained from the genome locations of these segments in the Ensembl database access via biomart (www.ensembl.org/info/data/biomart/index.html). For example, for the determination of the fraction of IgG B cells, the method described above is used to determine segment usage for each of the following segments: IGHG1, IGHG2, IGHG3, and IGHG4, and the fraction of B cells in this class is calculated as the sum of the fractions of cells obtained for each of these segments. The fractions of cells obtained for a segment is calculated using the model above as the absolute value of the difference between the estimate from the model for the particular segment and the preceding segment. This is described in detail further below. The same normalisation regions are used as for determining the total B cell fraction.

Due to sequencing noise (particularly when using WES data) it is desirable to have as wide a region as possible in the calculation of the normal baseline using V gene segments unlikely to be commonly lost in VDJ recombination. However, the more V gene segments chosen the more likely that B cell clonotypes exist where these gene segments have been excised by TRECs (such that the coverage in the region would then be influenced by VDJ recombination).

For the calculation of both the total B cell fraction and the class-specific B cell fractions, the following normalisation regions were used:
1. Before potential class switching IGH deletion events (from the start of the IGH locus to the end of the first class switching segment IGHA2):
   Hg38: chr14:105566277-105588395
   Hg19: chr14: 106779812-106879844
2. In the IGH constant region after any V(D)J recombination deletion events (final ~10k bases of the IGH locus (in particular, 10000 bases in Hg19 and 10032 bases in Hg38), including 13 V segments):
   Hg38: chr14: 106779812-106879844
   Hg19: chr14: 107188051-107288051
      These were chosen as regions within the IGH locus where there is no expected loss event from either class switching or VDJ recombination. In WGS data, all of these regions had appropriate base coverage. For WES data or data where noise and/or base coverage may be uneven, normalisation regions may be chosen from candidate regions where there is no expected loss event for example by comparing scores obtained with different normalisation regions to orthogonal metrics of B cell fraction such as e.g., RNA derived metrics. For example, a process as described in WO 2023/002046 may be used (such as e.g. a scheme as described on pages 56-57 and/or 14-15 of WO 2023/002046).

### GC Normalisation—WES Data

GC content is known to bias sequence coverage values. Thus, the methods described herein can comprise normalise the coverage values by GC content before calculation of the B cell fraction. To do this GC content is calculated at two scales, the local exon level where GC content for each IGH exon (GCₑₓₒₙ) is calculated and a larger macro scale (GC_{macro}). For this larger scale the GC content is calculated in 1000 bp windows across the entire IGH locus. A general additive model is then fitted to this data to create a smoothed value for the macro scale GC content across the IGH locus.

To normalise for GC content the coverage values of every base-pair within all IGH genes are put into the following linear model:

$$lm ( basepair ∼ G {C}_{e x o n} + G {C}_{e x o n}^{2} + G {C}_{m a c r o} + G {C}_{m}^{2} )$$

The residuals of this model are then taken as the new GC normalised coverage values. Unless stated otherwise all IGH B cell fractions used in these examples have been GC corrected.

Following GC correction the baseline of scores are re-adjusted by taking the median value at the beginning and end of the IGH locus (Hg19: chr14: 106779812-106879844 and 107188051-107288051) as well as the median value of the fitted generalised additive model (GAM) at the location of maximum VDJ recombination/or a particular CSR segment, depending on whether total B cell fraction or fraction of B cells in a particular class are being calculated. As it is theoretically impossible for the B cell fraction to be negative and by necessity for this to be so the log read depth ratio at chr14: 105865679-105939755 in Hg38 must be ≤0, the maximum of the two median values is taken as the new baseline and set to 0.

### GC Normalisation—WGS Data

The GC correction method described above was adapted to work on WGS instead of exons as described by a capture kit. The GC content was calculated at two scales, within 1000 bp windows (GC_{1000bp}) and a smoothed version as calculated using a GAM model across all 1000 bp segments (GC_{macro}), as explained above. The coverage values were then normalised for GC content by fitting and taking the residuals from the following linear model:

$$lm ( basepair ∼ G {C}_{1000 bp} + G {C}_{1000 bp}^{2} + G {C}_{m a c r o} + G {C}_{macro}^{2} )$$

This is identical to the one described in Example 1 except that the local GC content was calculated at the level of 1000 bp windows (GC_{1000bp}) rather than at the level of individual exons (GCₑₓₒₙ).

### Additional Quality Control—WGS Data

As a final step a robust quality control process identified any 100 bp segments within any part of the IGH locus, in particular within any of the V(D) genes or any of the class switch segments, that were outliers in terms of coverage either 1) across the entire cohort or 2) associated with a subset of the cohort and linked to known germline genomic variants. For the first step the mean GC corrected ratio of all the V(D)J genes were calculated for the set of all the GEL Lung cohort. This revealed segments with either extremely low coverage compared to the regions surrounding them or extremely high coverage. By fitting a GAM model to the mean GC corrected ratio, those above and below a certain threshold from the fitted line (+/−0.25) could be excluded. This worked well for TCRA but for other genes such as TCRB there were large segments clustered together that interfered with the fit of the GAM model. For these some clear outlier regions were initially removed manually, e.g. all regions with mean GC ratio above 0.25 for TCRB.

Following the removal of segments identified in this way, additional segments with biases were identified following a GWAS analysis using the PLINK software (Renteria et al., 2013). In particular, a GWAS analysis was conducted to identify any SNPs associated with the B cell fraction. Regions of genes with bias in coverage in certain exons were identified and flagged for removal. The scores were recalculated and the GWAS process was repeated to check for removal of the bias. The removed segments had either under or over coverage relative to their surrounding region associated with a particular germline genotype and caused certain variants within the genes to be strongly associated with B cell fraction purely due to this artifact. In these cases, the specific samples with the associated genotype were selected and the average GC corrected ratio calculated. Additional outlier segments were then identified and flagged for removal by the same procedure as described above and the PLINK/GWAS analysis was rerun to assure that there were no further artifact segments.

GWAS Analysis with PLINK

PLINK was run on a cohort of unrelated participants with WGS germline samples from blood and controlled with covariates for the first 20 genetic PCs, sex, age and disease type. The input of PLINK were the variants that had been pre-processed such that they had a MAF>0.001 in the entire cohort, passed QC filters including missingness and sufficient depth and underwent variant normalisation such as to make all multi-allelic variants bi-allelic as well as ensuring all variants were left aligned and parsimonious. In addition to these pre-set filters and QC before running PLINK we performed LD pruning in 500 kb windows with a R2 threshold of 0.2 as well as ensuring all variants had a MAF>0.001 in our cohort and a genotype missingness no greater than 0.2. Finally before running PLINK a Hardy-Weinberg test was performed with a threshold of 0.000001.

### Calculation of Confidence Intervals—GAM Model

95% confidence intervals for the IGH B cell fraction was calculated taking two factors into account, 1) the noise in the final baseline adjustment post GC corrections, 2) the uncertainty in fitting the GAM model used in the final calculation of the IGH B cell fraction.

To account for the noise in the baseline adjustment, the 95% confidence interval for the adjustment value was calculated as 1.96 times the standard deviation of the coverage values in the region used for normalisation. For the fitting of the GAM model, the R package ‘gratia’ (v0.5.1) was used to calculate the simultaneous confidence intervals (using the confint function). Finally these two sources of uncertainty were combined to generate the 95% confidence intervals.

Note that the segment model described below produces a ‘best fit’ result and there is no calculation of a confidence interval for this model.

### IGH B Cell Fraction Bias Due to Over-Estimated Tumour IGH Copy Number

Over-estimated values for IGH tumour copy number will lead to an inflated value for the IGH B cell fraction. This can occur due to a poor quality solution choice from copy number segmentation algorithm such as ASCAT. One indication that this is the case is if the calculated IGH B cell fraction exceeds the value of 1−tumour purity. In these cases the given copy number solution is deemed unreliable and instead the naive estimate for B cell fraction that assumes that the IGH tumour copy number is 2 is used as an alternative solution.

### Segment Usage

Similarly, the method was also applied to study segment usage, using the above method but using for the value of r_{B} the estimate from the segment model for the particular segment corresponding to the particular gene under investigation (in particular, the absolute value of the difference between the estimate from the segment model for the particular segment and the preceding segment—i.e. the drop or increase, depending on whether a V or J segment is considered, at the start of the segment under investigation, as illustrated on FIG. 6C). Thus, the normalisation regions described above are used together with the segments equivalent to the “Maximum V(D)J recombination region” for each gene (provided as gene hgnc symbol-start_hg38-end_hg38) in Table 1 below. In this example, the segment model was used when studying segment usage, with the estimate from the model for the corresponding segment representing the value of r_{B}. However, the same regions provided in Table 1 can be used in combination with a GAM, preferably when coverage allows for studying of these regions, by estimating a summarised value for each of a plurality of segments to be investigated (e.g. median value) and determining the difference between consecutive segments as the r_{B} for the second of a pair of consecutive segments.

### Calculation of Class Switched B Cell Fractions

The IGH locus contains 9 genes downstream of the V(D)J region, in the C region, μ—IgM, δ—IgD, γ₃—IgG3, γ₁—IgG1, α₁—IgA1, γ₂—IgG2, γ₄—IgG4, ε—IgE, α₂—IgA2 (FIG. 7B). Recombination and excision of a DNA segment containing 1-8 of these genes generates the 3 B cell classes, IgA, IgE, and IgG, while IgM- and IgD-class naïve B cells contain all 9 exons. To identify the proportion of the total B cell fraction of a particular class, an adapted constrained segment model is used. the model is constrained such that:
1. breakpoints for deletion events are only possible at the end of the μ, δ, γ₃, γ₁, α₁, γ₂, γ₄, ε, and α₂ exons within the constant region (i.e. breakpoints for deletion events are only possible at the end of the IGHA2, IGHE, IGHG4, IGHG2, IGHA1, IGHG1 or IGHG3 gene segment locus). The genomic locations used for predicting class switch segments for each of the IGH contact region exons are given in Table 2.
2. The end of the IGHM locus represents the end of any class switching deletion event, and the log read depth ratio is assumed to return to 0. This constraint is applied by requiring that a segment from the end of the IGHM segment to the first J segment (IGHJ6) is set to 0. The coordinates of this segment are 105856218-105863258 (Hg38).
3. The number of class switched B cells must be less or equal to the total number of B cells, so the log read depth ratio after the IGHG3 locus must be less or equal to the log read depth ratio at the point of maximum V(D)J recombination. In other words, the value of the model at the IGHD and IGHM segments must be equal or less than the value of the model determined at the location of maximum VDJ recombination.

Fitting the constrained linear model with the class switching and V(D)J restraints leads to predicted class switching segment usage. Thus, changes in the log read depth ratio (r_{B}) in each these segments are then used to calculate the fraction of a particular class.

TABLE 1
Locations of regions (hg38) used for single segment calculation
of r_{B} in the VDJ region in the IGH locus (i.e. IGH segments).

IGHA2-105583731-105588395; IGHE-105597691-105601728; IGHG4-105620506-
105626066; IGHG2-105639559-105644790; IGHGP-105664633-105669843; IGHA1-
105703995-105708665; IGHEP1-105721794-105722527; IGHG1-105736343-
105743071; IGHG3-105764503-105771405; IGHD-105836765-105845677; IGHM-
105851705-105856218; IGHJ6-105863198-105863258; IGHJ3P-105863416-
105863465; IGHJ5-105863814-105863862; IGHJ4-105864215-105864260; IGHJ3-
105864587-105864635; IGHJ2P-105864793-105864852; IGHJ2-105865199-
105865250; IGHJ1-105865407-105865458; IGHD7-27-105865551-105865561;
IGHJ1P-105865624-105865678; IGHD1-26-105881034-105881053; IGHD6-25-
105881539-105881556; IGHD5-24-105883903-105883922; IGHD4-23-105884870-
105884888; IGHD3-22-105886031-105886061; IGHD2-21-105888551-105888578;
IGHD1-20-105891191-105891207; IGHD6-19-105891699-105891719; IGHD5-18-
105893542-105893561; IGHD4-17-105894508-105894523; IGHD3-16-105895634-
105895670; IGHD2-15-105897957-105897987; IGHD1-14-105900638-105900654;
IGHD6-13-105901142-105901162; IGHD5-12-105902649-105902671; IGHD4-11-
105903616-105903631; IGHD3-10-105904497-105904527; IGHD3-9-105904681-
105904711; IGHD2-8-105907211-105907241; IGHD1-7-105909907-105909923;
IGHD6-6-105910410-105910427; IGHD5-5-105912257-105912276; IGHD4-4-
105913222-105913237; IGHD3-3-105914359-105914389; IGHD2-2-105916826-
105916856; IGHD1-1-105919502-105919518; IGHV6-1-105939756-105940253;
IGHVII-1-1-105945210-105945389; IGHV1-2-105986582-105987083; IGHVIII-2-1-
106001534-106001824; IGHV1-3-106005095-106005574; IGHV4-4-106011922-
106012420; IGHV7-4-1-106025145-106025630; IGHV2-5-106037902-106038365;
IGHVIII-5-1-106039666-106039764; IGHVIII-5-2-106045600-106045860; IGHV3-6-
106055549-106055998; IGHV3-7-106062151-106062683; IGHV3-64D-106088122-
106088573; IGHV5-10-1-106107972-106108464; IGHV3-11-106116635-106117204;
IGHVIII-11-1-106120207-106120473; IGHV1-12-106122420-106122709; IGHV3-13-
106129540-106130072; IGHVIII-13-1-106142287-106142577; IGHV1-14-106145891-
106146131; IGHV3-15-106153624-106154163; IGHVII-15-1-106163583-106163802;
IGHV3-16-106165205-106165730; IGHVIII-16-1-106170749-106171052; IGHV1-17-
106174342-106174760; IGHV1-18-106184901-106185394; IGHV3-19-106196700-
106196990; IGHV3-20-106210936-106211453; IGHV3-21-106235064-106235594;
IGHV3-22-106257762-106258223; IGHVII-22-1-106263362-106263626; IGHVIII-22-2-
106264688-106264715; IGHV3-23-106268606-106269140; IGHV1-24-106276548-
106277043; IGHV3-25-106289029-106289479; IGHVIII-25-1-106293568-106293808;
IGHV2-26-106301396-106301862; IGHVIII-26-1-106309417-106309666; IGHVII-26-2-
106314651-106314975; IGHV7-27-106317823-106318236; IGHV4-28-106324254-
106324760; IGHVII-28-1-106329432-106329684; IGHV3-32-106331106-106331563;
IGHV3-30-106335082-106335613; IGHVII-30-1-106342680-106342980; IGHV3-30-2-
106344385-106344833; IGHV4-31-106349283-106349792; IGHVII-30-21-106354448-
106354704; IGHV3-29-106356145-106356591; IGHV3-33-106359793-106360324;
IGHVII-33-1-106367385-106367664; IGHV3-33-2-106369107-106369546; IGHV4-34-
106373663-106374145; IGHV7-34-1-106377300-106377733; IGHV3-35-106389392-
106389858; IGHV3-36-106392774-106393231; IGHV3-37-106396676-106397114;
IGHV3-38-106410493-106411021; IGHVIII-38-1-106418018-106418299; IGHV4-39-
106421711-106422218; IGHV7-40-106425359-106425654; IGHVII-40-1-106440950-
106441007; IGHV3-41-106443133-106443583; IGHV3-42-106463256-106463691;
IGHV3-43-106470264-106470800; IGHVII-43-1-106472791-106473000; IGHVIII-44-
106478006-106478393; IGHVIV-44-1-106489345-106489756; IGHVII-44-2-
106494134-106494383; IGHV1-45-106506996-106507491; IGHV1-46-106511117-
106511856; IGHVII-46-1-106515818-106515854; IGHV3-47-106518582-106519027;
IGHVIII-47-1-106531141-106531471; IGHV3-48-106537810-106538344; IGHV3-49-
106556936-106557477; IGHVII-49-1-106564375-106564598; IGHV3-50-106566117-
106566555; IGHV5-51-106578744-106579236; IGHV8-51-1-106583501-106583807;
IGHVII-51-2-106584718-106584976; IGHV3-52-106586376-106586826; IGHV3-53-
106592676-106593347; IGHVII-53-1-106599670-106599924; IGHV3-54-106601346-
106601792; IGHV4-55-106606101-106606551; IGHV7-56-106609762-106610196;
IGHV3-57-106618894-106619173; IGHV1-58-106622357-106622855; IGHV4-59-
106627249-106627825; IGHV3-60-106631197-106631653; IGHVII-60-1-106637718-
106637973; IGHV4-61-106639119-106639657; IGHV3-62-106643142-106643585;
IGHVII-62-1-106650551-106650785; IGHV3-63-106652237-106652681; IGHV3-64-
106657725-106658258; IGHV3-65-106666092-106666532; IGHVII-65-1-106671772-
106672073; IGHV3-66-106675017-106675544; IGHV1-67-106680603-106681042;
IGHVII-67-1-106686517-106686562; IGHVIII-67-2-106687113-106687200; IGHVIII-
67-3-106692657-106692888; IGHVIII-67-4-106695102-106695397; IGHV1-68-
106703846-106704286; IGHV1-69-106714684-106715181; IGHV2-70D-106723574-
106724093; IGHV3-69-1-106728163-106728615; IGHV1-69-2-106737110-
106737547; IGHV1-69D-106762092-106762588; IGHV2-70-106770577-106771020;
IGHV3-71-106775157-106775618; IGHV3-72-106790694-106791233; IGHV3-73-
106802694-106803233; IGHV3-74-106810442-106811131; IGHVII-74-1-106821174-
106821414; IGHV3-75-106823687-106824147; IGHV3-76-106827869-106828163;
IGHVIII-76-1-106831767-106832052; IGHV5-78-106851123-106851417; IGHVII-78-1-
106865627-106865895; IGHV3-79-106867660-106868092; IGHV4-80-106872783-
106873186; IGHV7-81-106874583-106875071; IGHVIII-82-106879563-106879812.

TABLE 2
Locations of IGH class switching segments on either hg38 or
hg19 co-ordinates, used for calculating r_{B} in the CSR region.

| Gene symbol | Hg38 start | Hg38 end | Hg19 start | Hg19 end |
|---|---|---|---|---|
| IGHA2 | 105583731 | 105588395 | 106053226 | 106054732 |
| IGHE | 105597691 | 105601728 | 106064224 | 106068065 |
| IGHG4 | 105620506 | 105626066 | 106090687 | 106092403 |
| IGHG2 | 105639559 | 105644790 | 106109389 | 106111127 |
| IGHA1 | 105703995 | 105708665 | 106173457 | 106175002 |
| IGHG1 | 105736343 | 105743071 | 106202680 | 106209408 |
| IGHG3 | 105764503 | 105771405 | 106235439 | 106237742 |
| IGHD | 105836765 | 105845677 | 106303099 | 106312010 |
| IGHM | 105851705 | 105856218 | 106320349 | 106322323 |

Fitting the constrained linear model with the class switching and V(D)J restraints leads to predicted class switching segment usage. Specific class switched B cells can then be calculated in the following ways:
igG B cells=IGHG1+IGHG2+IGHG3+IGHG4
igA B cells=IGHA1+IGHA2
igE B cells=IGHE
Non-class switched B cells (igM/D B cells)=Total B cell fraction−(igA+igG+igE)
   where IGHG1 refers to the segment usage for the IGHG1 segment (difference in absolute value of r_{B} between the IGHG1 segment and the IGHG3 segment), IGHG2 refers to the segment usage for the IGHG2 segment (difference in absolute value of r_{B} between the IGHG2 segment and the IGHA1 segment), IGHG3 refers to the segment usage for the IGHG3 segment (difference in absolute value of r_{B} between the IGHG3 segment and the IGHD segment), IGHG4 refers to the segment usage for the IGHG4 segment (difference in absolute value of r_{B} between the IGHG4 segment and the IGHG2 segment), IGHA1 refers to the segment usage for the IGHA1 segment (difference in absolute value of r_{B} between the IGHA1 segment and the IGHG1 segment), IGHA2 refers to the segment usage for the IGHA2 segment (difference in absolute value of r_{B} between the IGHA2 segment and the IGHE segment), IGHE refers to the segment usage for the IGHE segment (difference in absolute value of r_{B} between the IGHE segment and the IGHG4 segment), and Total B cell fraction refers to the B cell fraction calculated using the location of maximum VDJ recombination in the IGH locus as explained above.

In other words, the fraction of igE B cells is calculated as the fraction of cells that have a deletion breakpoint between the IGHE segment and the IGHG4 segment. The fraction of igA B cells is calculated as the sum of: (i) the fraction of cells that have a deletion breakpoint between the IGHA1 segment and the IGHG1 segment, and (ii) the fraction of cells that have a deletion breakpoint between the IGHA2 segment and the IGHE segment. The fraction of igG B cells is calculated as the sum of: (i) the fraction of cells that have a deletion breakpoint between the IGHG1 segment and the IGHG3 segment, (ii) the fraction of cells that have a deletion breakpoint between the IGHG3 segment and the IGHD segment, (iii) the fraction of cells that have a deletion breakpoint between the IGHG2 segment and the IGHA1 segment, (iv) the fraction of cells that have a deletion breakpoint between the IGHG4 segment and the IGHG2 segment. Each such fraction is calculated using a value of r_{B} that is the absolute value of the difference between the summarised read depth ratio for the respective segments.

In summary, for the calculation of both total B cell fraction (f^{tot}) and class specific B cell fraction (f^{class}) the following process may be used:
1. The read depth is calculated along the IGH locus from either WES or WGS data (preferably, WGS data is used as it has better coverage in the class switching regions—currently widely available WES data may have insufficient coverage to calculate individual segment usage in the CSR region, although a total class switched B cell proportion can be calculated and specific WES platforms may have better coverage of this region);
2. Outlier segments/regions are optionally removed;
3. A read depth ratio profile is calculated by normalising the read depth by the median read depth in normalisation regions proximal to the IGH gene (or within the IGH gene) that are not expected to undergo either CSR or VDJ recombination;
4. A model is fitted to the log of the profile calculated in step 3—this can be done using a GAM or segment-based model (also referred to as “piecewise constant model”)—when using a GAM model, two separate models are preferably fitted to the VDJ and CSR regions; when a segment based model is used, this is fitted using the following constraints:
   a. In the VDJ region: the segments align with the locations of the V and J segments, the read depth ratio should start from 0 and from there must be monotonically decreasing until the point of maximum V(D)J recombination following the final V segment, and the read depth must be monotonically increasing until reaching 0 in the J segments;
   b. In the CSR region: the segments align with the locations of exons μ, δ, γ3, γ1, α1, γ2, γ4, ε, and α2 exons within the constant region, the log read depth ratio must return to 0 at the end of the IGHM locus, and the log read depth ratio after the IGHG3 locus must be less or equal to the log read depth ratio at the point of maximum V(D)J recombination.
5. A total B cell fraction is calculated using either equation (3) or equation (7), where r_{B} is the log read depth obtained at step 4 for a region/segment that is designated as the region of maximum VDJ recombination.
6. Segment usage for each of segments IGHA2, IGHE, IGHG4, IGHG2, IGHA1, IGHG1 or IGHG3 (or corresponding summarised values from a smooth model) are calculated using either equation (3) or equation (7), where r_{B} is the absolute difference in log read depth obtained at step 4 for the segment considered compared to the preceding segment (when using a segment model) and/or a total class switched B cell fraction is calculated using either equation (3) or equation (7), where r_{B} is the log read depth obtained at step 4 for a region/segment that is designated as the region of maximum CSR recombination.
7. Class specific B cell fractions are obtained based on the segment usage values obtained at step 6, and the total B cell fraction obtained at step 5.

### Example 2: Inferring T and B Cell Fraction from Whole Genome Sequencing (WGS) Data Reveals Immune Dysregulation in Circulating Blood and Cancer

Circulating immune cells have long been used as a marker for general inflammation. In the context of cancer, the neutrophil-to-lymphocyte ratio (NLR), has been shown to be prognostic in many disease settings. Much research focus, however, has been on the importance of tumour-infiltrating rather than circulating immune cells, and disparate methods of measuring both simultaneously has prohibited a combined analysis.

The present inventors demonstrated in Bentham et al. 2021 how the T cell fraction of a tissue or blood sample can be measured from DNA sequencing data using a signal from V(D)J recombination within the T cell receptor alpha (TCRA) locus from whole exome sequencing samples (WES). In WO 2023/002046, the inventors further demonstrated that the approach can be applied to whole genome sequencing data (WGS), enabling T cell fraction from the TCRA, TCRB and TCRG loci, as well as B cell fraction from the IGH locus to be measured. In the present work, the present inventors show that the approach can be modified to measure B cell fractions with class switching predictions. In particular, they introduced a measure of B cell fraction from the IGH locus on chromosome 14, along with class switching prediction to predict the number of igA, igG or igE antibody producing B cells (see FIG. 7B).

They use the multi-omic TRACERx dataset to extensively validate the methods (including all of TCR cell fractions, B cell fraction, class switching predictions, repertoire diversity and segment usage—as illustrated on FIG. 7A), demonstrating that these scores are accurate reflections of the immune microenvironment, maintain accuracy on down-sampled samples to a depth of 5× and have utility in the investigation of BCR repertoire diversity.

They further apply the methods to the 100,000 genome project (100KGP) WGS pan-cancer cohort of ~15,000 tumours with matched germline blood samples across 20 cancer types with no existing orthogonal immune data, demonstrating their clinical value.

### Results

An example output of the methods for the IGH locus is shown on FIG. 7C, along with a visual representation of estimated clonotype diversity of the IGHV segment usage, as well as the percentage of predicted class-switched B cells.

### Validation Using Matched RNAseq TRACERx Data

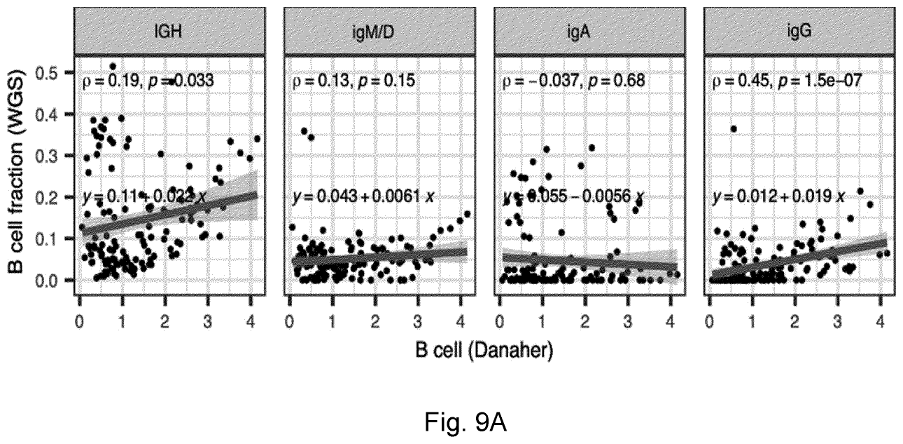

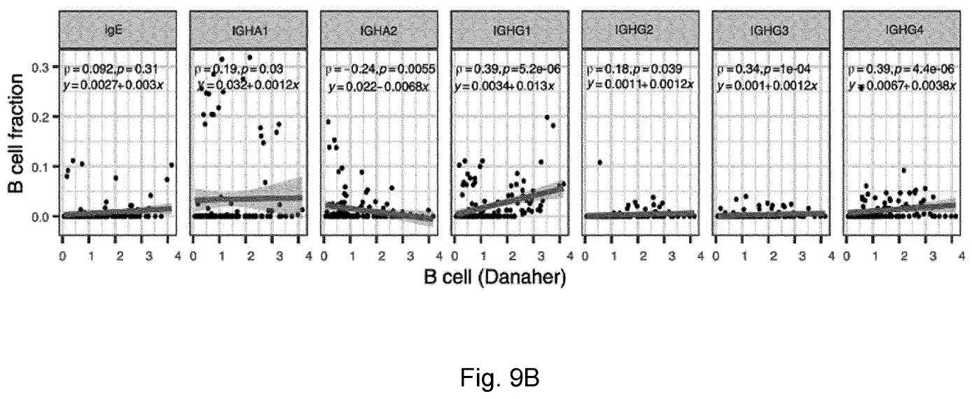

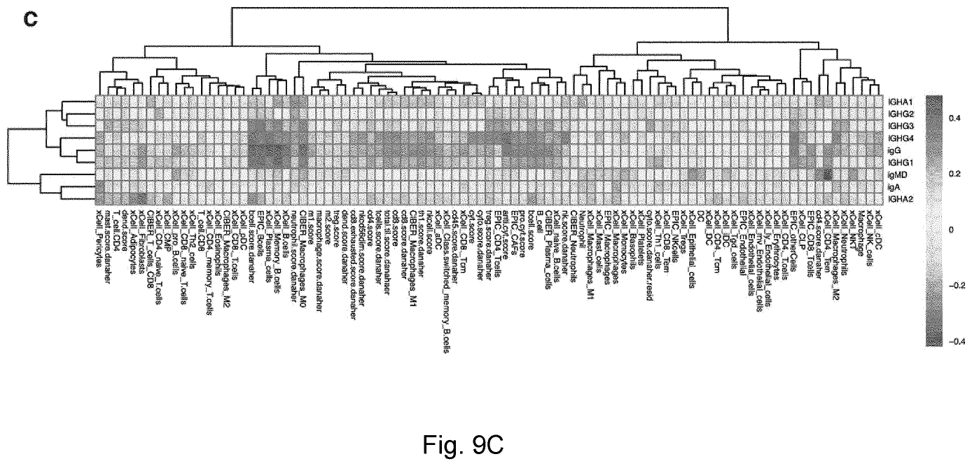

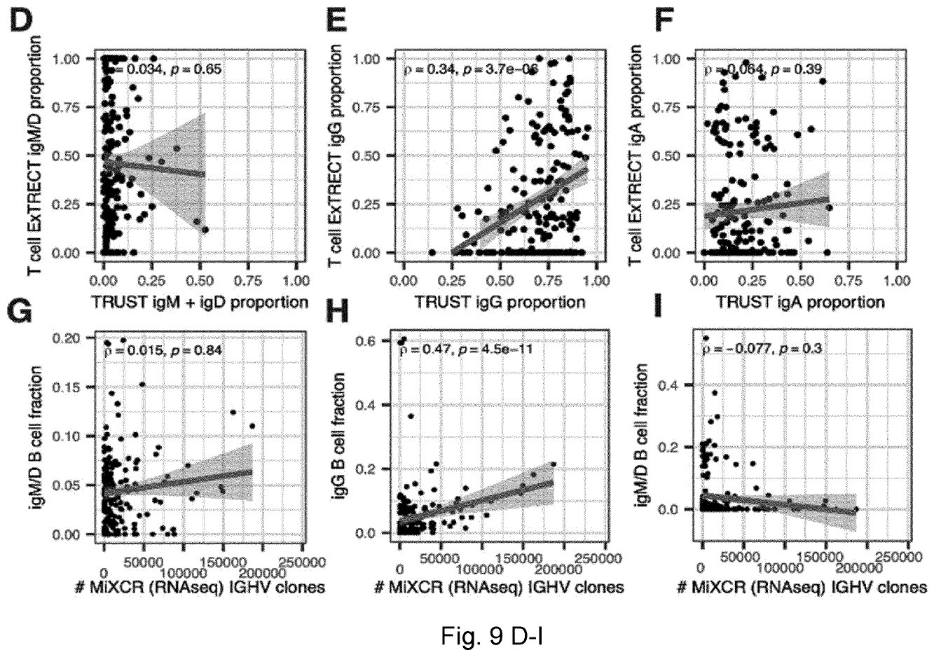

To validate the B cell fraction scores the inventors used the TRACERx100 RNAseq data set. They found a significant but weak correlation with the B cell Danaher score and total IGH B cell fraction (ρ=0.19, P=0.037, FIG. 9A). This association surprisingly was found to be solely from the igG B cell fraction and not the other class or non-class switched B cells (igG: ρ=0.45, P=1.1e-07; igA: ρ=0.037, P=0.68, igM/D: ρ=0.13, P=0.16, FIG. 9A), at an individual predicted class switched segment level significant positive correlation was found with all individual igG segments and IGHA1 but not IGHA2 or igE (igE: ρ=0.092, P=0.31, IGHA1: ρ=0.19, P=0.03, IGHA2: ρ=−0.24, P=0.0055, IGHG1: ρ=0.39, P=5.2e-6, IGHG2: ρ=0.18, P=0.039, IGHG3: ρ=0.34, P=1e-4, IGHG4: ρ=0.39, P=4.4e-6, FIG. 9B), suggesting that the Danaher B cell signature is coming almost entirely from igG B cells.

To identify RNA signatures correlated to the class switching fractions the inventors correlated these fractions with different RNA signatures produced by Danaher (Danaher et al. 2018), TIMER (Li et al., 2017), CIBERSORT (Newman et al. 2015), xCell (Newman et al. 2015) and Davoli (Davoli et al. 2017) (FIG. 9C). They found that fractions related to IGHA1, IGHG2, IGHG3, IGHG4, igG and IGHG1 were all correlated strongly to multiple B cell related signatures. igM/D in contrast was most strongly negatively correlated to the xCell signature for CD4 effector memory T cells, while IGHA2 was negatively correlated to many B cell scores and most strongly negatively to the xCell signature for fibroblasts. This data indicates that some class switched B cells are very transcriptionally silent and do not contribute much to RNAseq, and/or that existing B cell gene signatures are being trained on igG B cell expression patterns. The correlation to other signatures indicates that different classes of B cells are associated with different immune microenvironment states. Therefore, the quantification of class specific proportions may provide crucial information about the tumour microenvironment, that could help guide treatment and prognosis.

As an orthogonal method the inventors also correlated igA, igG and igM/D proportions to those calculated using the TRUST (Hu et al. 2019) method from the RNAseq data (FIG. 9D-F) as well as the number of unique IGHV clones identified from MiXCR run on the RNAseq data (FIG. 9G-I). In this they only identified a significant correlation with igG for both the TRUST igG calls (FIG. 9E: ρ=0.34, P=3.7e-06) and the number of IGHV clones identified from MiXCR (FIG. 9H: ρ=0.47, P=4.5e-11). Overall these data show a discordance between B cell predictions from DNA compared to RNA for all but igG B cells. Suggesting that igM/D B cells in lung tissues may predominantly be in a quiescent and therefore transcriptionally silent state. This further indicates that the methods described herein may be usable to identify the proportion of quiescent B cells in each of one or more specific classes, by comparing fractions obtained using the methods described herein to corresponding metrics derived from expression data (e.g. RNAseq metrics). For examples, samples where DNA-derived score(s) (obtained using methods as described herein) is high but the corresponding RNA-derived score(s) is/are low may be indicative of the sample being enriched in quiescent B cells.

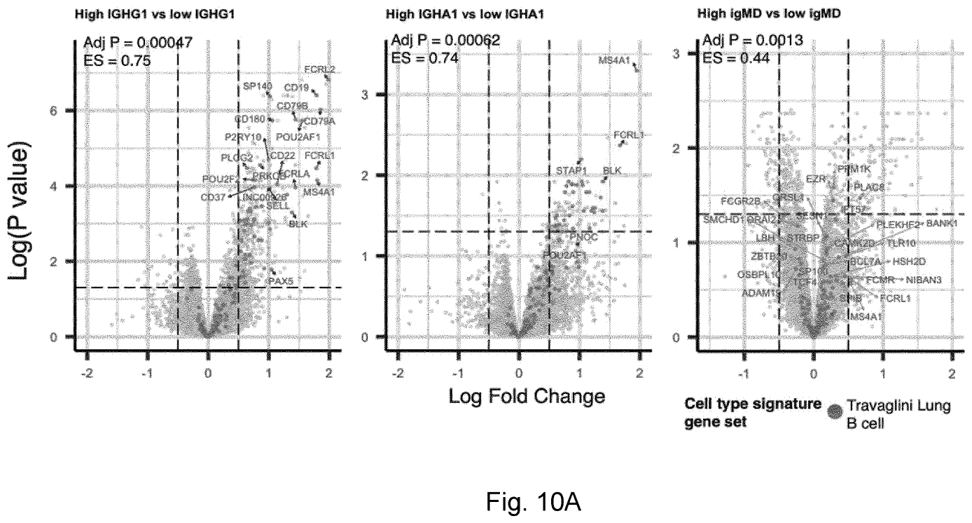

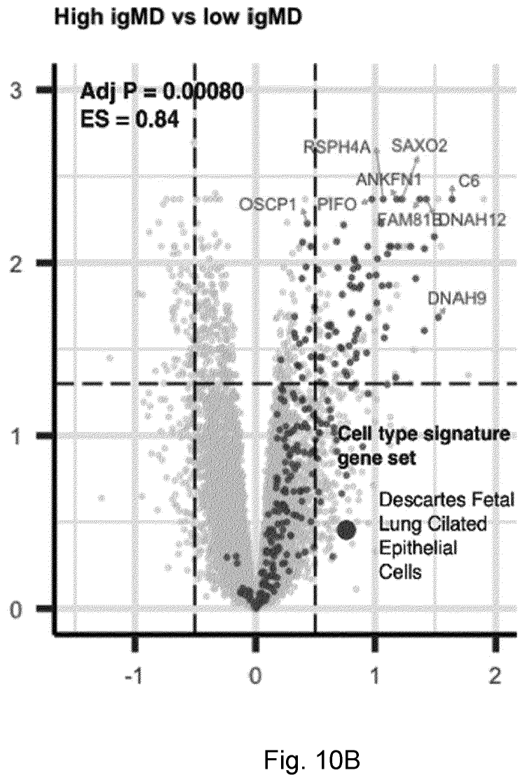

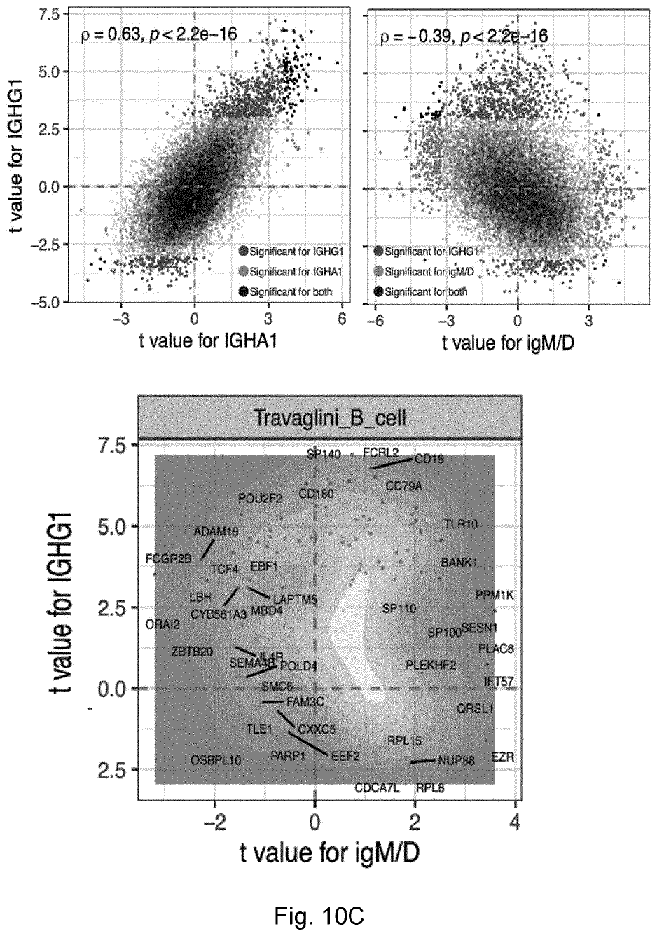

To gain insight into the differences between the other class switched fractions the inventors ran a differential expression analysis using limma voom (Law et al. 2014) for samples in the TRACERx100 data set with high versus low fractions (based on the higher of the median or 0.01) for the igM/D, IGHA1 and IGHG1 fractions (FIG. 10A). For all three they then ran a gene set enrichment analysis using the Molecular Signatures Database (MSigDB) C8 collection (Liberzon et al. 2011) of 704 cell type signature gene sets, derived from single cell RNA sequencing experiments. Highlighted in FIG. 10A are the genes from the Travaglini Lung B cell gene set (Travaglini et al. 2020) that was found to be significant in all three comparisons (IGHG1: adj P=0.00047, ES=0.75; IGHA1: adj P=0.00062, ES=0.74; igM/D: adj P=0.0013, ES=0.44). The effect size was however much higher in the IGHG1 and IGHA1 comparisons with top hits for both including MS4A1, the B cell surface molecule also known as CD20. Samples with high igM/D fractions were found to be strongly co-enriched with other stromal cell types with the top GSEA gene set in the igM/D comparison found to be lung ciliated epithelial cells (adj P=0.00080, ES=0.84, FIG. 10B).

Examining the t values for each individual gene from each limma voom analysis, there was a strong correlation between the IGHG1 and IGHA1 analysis (FIG. 10C, top left panel: ρ=0.63, P<2.2e-16) suggesting a similar regulation of up or down differentially expressed genes. There were some notable differences including FABP4, a gene expressed in adiopocytes and macrophages being highly upregulated in high IGHA1 and not high IGHG1 samples, suggesting different cell populations associated with high levels of IGHA1 B cells. Comparing IGHG1 with igM/D t values there was instead a significant negative correlation (FIG. 10C, top right panel: ρ=−0.39, P<2.2e-16). The majority of B cell related genes were however upregulated in both comparisons (FIG. 10C bottom panel), with some notable exceptions such as FCGR2B the inhibitory receptor for the Fc region of igG having a positive t value in IGHG1 but a negative one for igM/D. The opposite was seen in EZR, the gene encoding the cytoplasmic peripheral protein ezrin and involved in regulation of BCR signalling with a positive t value in igM/D but a negative one in IGHG1. This data suggests a possible transcriptionally quiet non-class switched B cell population may be associated with samples replete with lung ciliated cells. These igM/D B cells could represent B cells that are in a dormant state while being co-localised to the respiratory cilia.

Taken together these results demonstrate that methods described herein are able to be applied to WGS data to accurately quantify B cell fraction.

### Accurate Measurement of T and B Cell Fraction at Low Sample Coverage

To explore the minimum depth required for accurate measurement of B cell fraction using WGS data, the inventors performed nested downsampling to the TRACERx100 cohort, with each sample initially being downsampled to 60×, and then downsampled further to 50, 40, 30, 20, 10, 5, 2, 1, 0.5, and finally 0.1×.

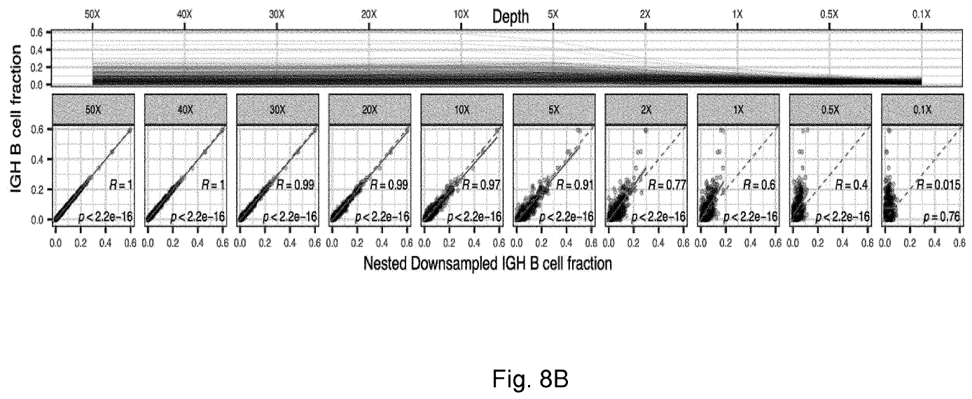

Strikingly, they found that the methods described herein still provided accurate measurements for B cell content at depths as low as 5× for the IGH locus (R=0.91, P<2.2e-16, see FIG. 8A). For the downsampled samples with coverage depths below 2×, however, the calculated B cell content begins to lose its range (FIG. 8B top panel), with it being less able to distinguish higher from lower values. This corresponds to a gradual loss of signal (60× vs 1×: R=0.6, P<2.2e-16; 60× vs 0.5×: R=0.4, P<2.2e-16; 60× vs 0.1×: R=0.015, P=0.76; FIG. 8B bottom panel).

These results show that the important factor for accurate B cell content calculation is not depth of coverage, but uniform coverage across the entire TCR of IGH locus. Uniformity of coverage, also referred to as “evenness” can be quantified as described in Oexle (2016). For example, evenness may be calculated as 1 minus the integral from 0 to 1 over the cumulative distribution function F(x) of the normalized coverage x (raw coverage divided by mean coverage), or 1 minus the integral from 0 to 1 over the probability density distribution f(x) times 1−x.

The T and B Cell Landscape in Blood and Tumours within the 100KGP Cohort

The inventors applied the full pipeline described on FIG. 7A including the methods described herein to the entire 100KGP cohort resulting in calculated immune fraction scores from 92,905 WGS bam files.

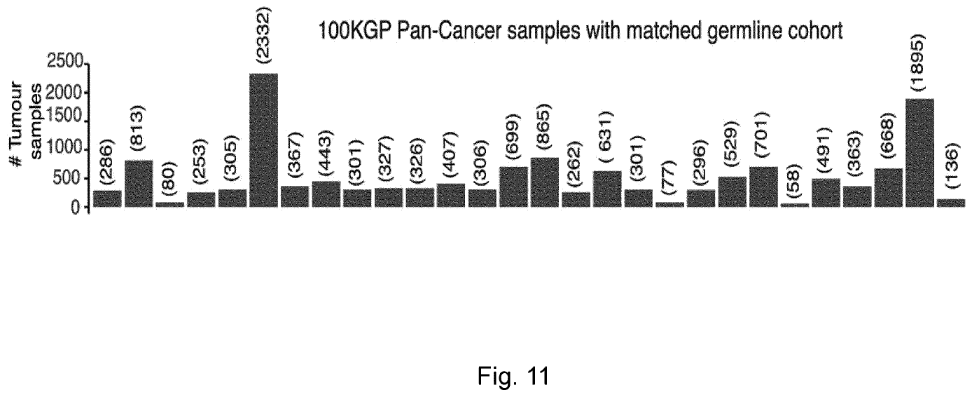

The 100KGP pan cancer cohort analysed contained data from 15,114 participants representing 28 distinct cancer histologies (see FIG. 11) that contained WGS samples from more than 50 participants. 14,576/15,113 of the analysed participants had a tumour WGS sample and 14,664/15,113 participants had a WGS blood sample. 13,941 participants had both a tumour and matched blood sample. With the discrepancy being due to a number of haematological cancer patients having matching germline samples from either saliva or normal tissue as well as the removal of tumour samples from participants that had multiple samples sequenced with unclear annotation.

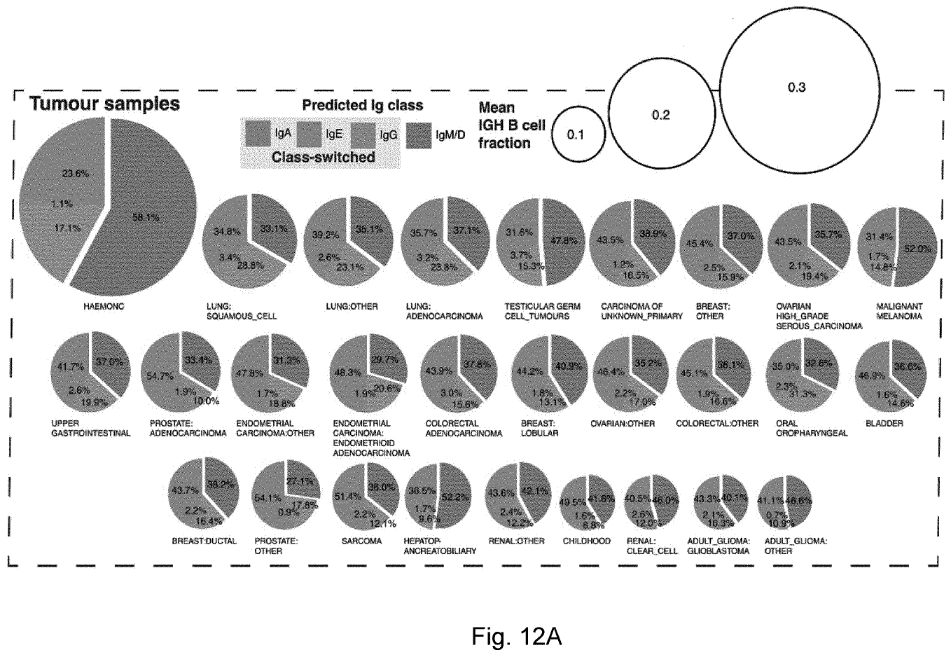

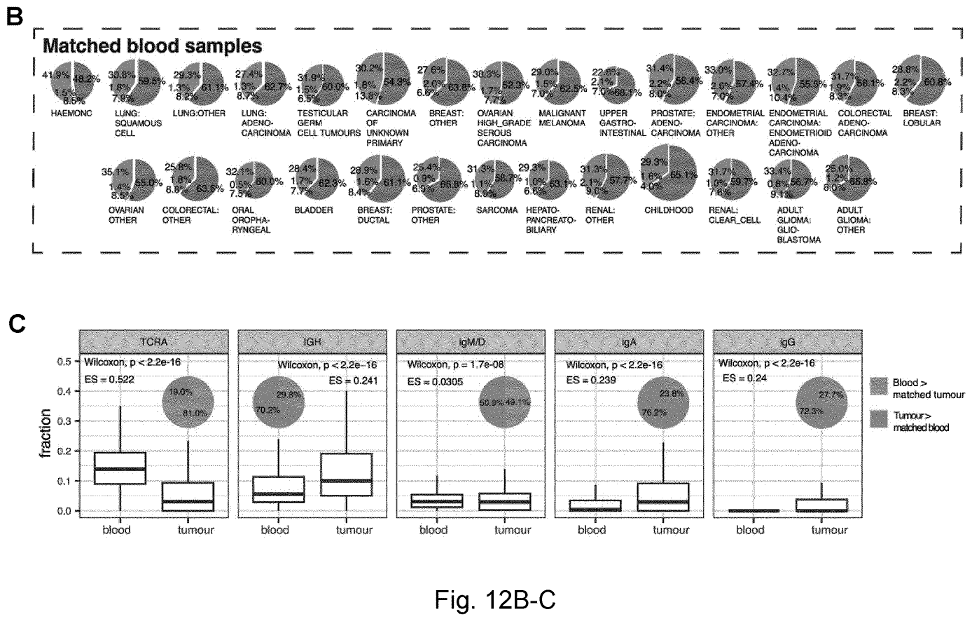

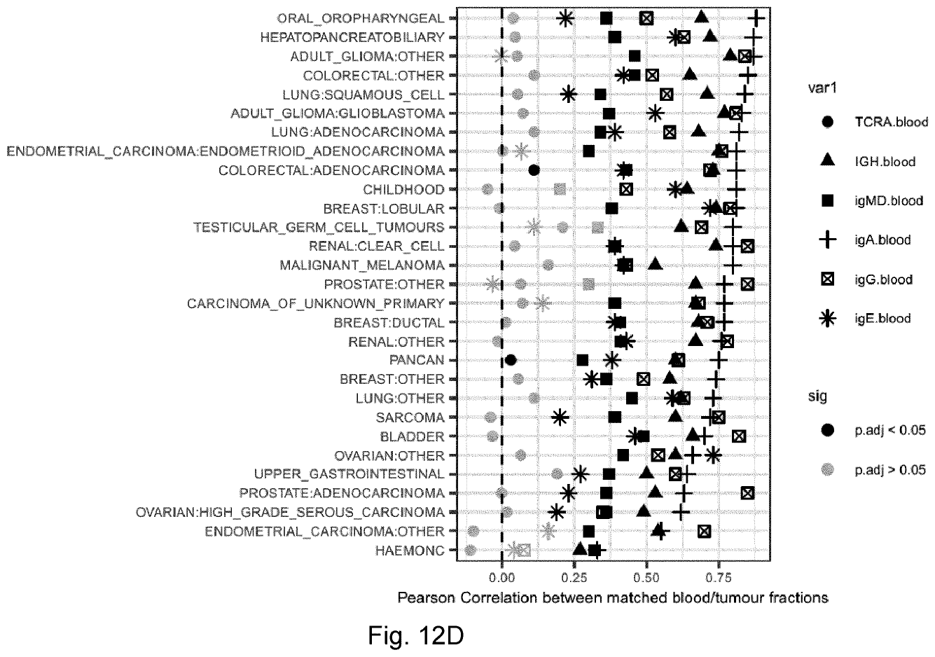

The inventors observed significant differences in the B cell landscape across both cancer and blood (FIGS. 12A and B), (Kruskal-Wallis, both P<2.2e-16), with the additional resolution of class switching predictions for every cohort. The methods described herein afforded them the opportunity to evaluate differences in B cell class switching between different tumours, and matched blood controls. They observed that B cells were significantly enriched in tumour samples compared to blood (ES=0.241, P<2.2e-16, 70.2% participants higher in tumour, FIG. 12C). Moreover, this effect was primarily driven by enrichment in igA and igG B cells within tumours compared to blood (igA: ES=0.239, P<2.2e-16, 76.2% participants higher in tumour; igG: ES=0.24, P<2.2e-16, 72.3% participants higher in tumour, FIG. 12C), with little alteration in non-classed switched B cells observed (igM/D: ES=0.0305, 50.9% participants higher in tumour, FIG. 12C). Thus highlighting key differences in the function and make-up of circulating and tumour infiltrating B cells.

To examine the extent blood and tumour immune fractions are related the inventors computed the Pearson correlation between them across the entire pan cancer cohort and each individual subtype (FIG. 12D). The correlation in IGH B cell fraction between blood and tumour was strongly significant in all histologies, mainly being driven by a very strong correlation in the igA B cell fraction (Pan cancer: R=0.75, adj P<2.2e-16) compared to a still significant but much lower fraction for non-class switched igM/D B cell fractions (Pan cancer: R=0.28, adj P<2.2e-16).

Circulating Immune Cells More Prognostic of Survival than Tumour Infiltrating Immune Cells

The presence and quantity of infiltrating tumour immune cells have been previously shown to be prognostic for survival in many cancer types. Similarly, circulating immune cell data from blood counts have been widely demonstrated to have prognostic importance. Using the methods described herein to measure both the circulating and tumour infiltrating B cell fraction from WGS simultaneously at the time of sample collection, the inventors were able to directly compare the prognostic value of both measurements in a pan cancer setting.

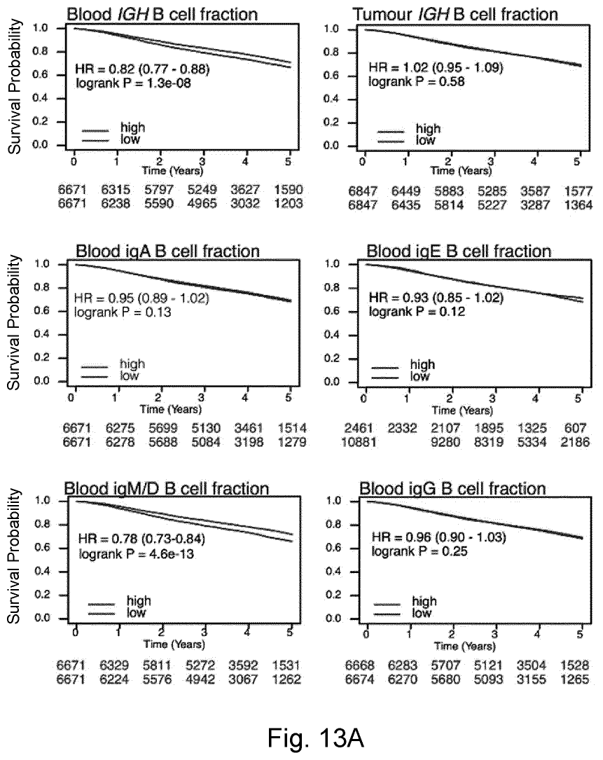

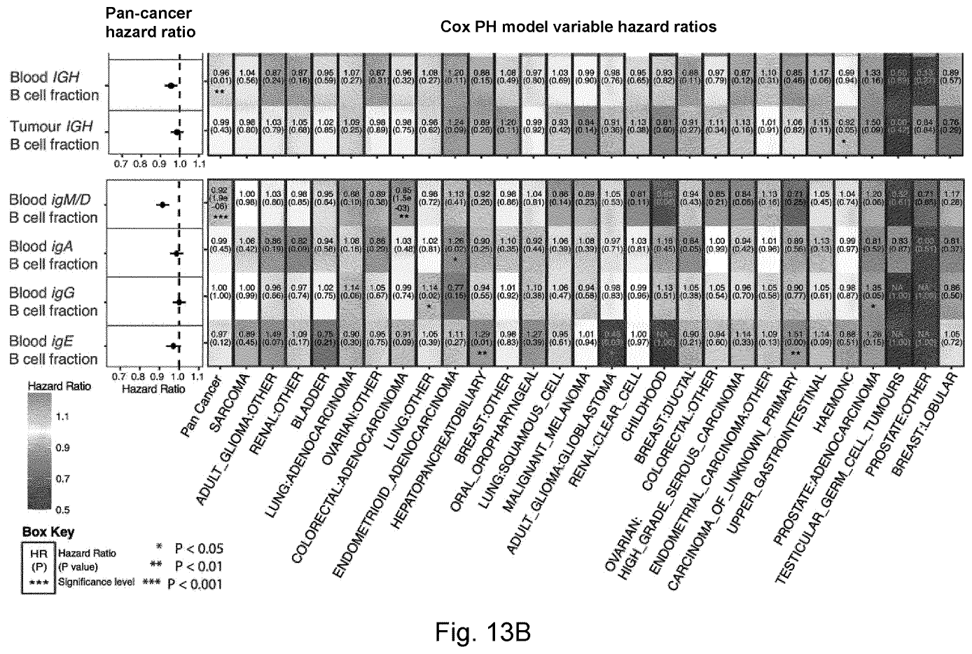

Across the pan cancer cohort, higher IGH B cell fraction was found to be significantly associated with improved overall survival outcomes, with the high fraction group being more significant in blood (FIG. 13A, HR=0.82, logrank P=1.3e-08) than in tumour tissue where it did not reach significance (FIG. 13A, HR=1.02, logrank P=0.6).

Splitting up IGH B cell fraction by the class switched fractions revealed that patients with high non-class switched blood igM/D B cell fraction were associated with improved prognosis (FIG. 13A: igM/D: HR=0.79, logrank P=4.6e-13), while patients with lower blood igG, igA or igE B cell fraction had no significant difference.

To explore the relationship of immune cell fraction with other clinical features and its heterogeneity across different cancer types, Cox proportional hazard (CoxPH) models were fitted, controlling for age, sex, and whether the patient received chemotherapy pre-surgery and sample collection. This received chemotherapy factor was included both as a surrogate for the incomplete cancer stage data within the 100KGP pan cancer cohort since neoadjuvant chemotherapy is mainly used on higher stage tumours and also due to the confounding effect chemotherapy has on the immune system and thereby the quantity of T and B cells.

Examining IGH B cell fraction in the CoxPH model showed that increased blood IGH B cell fraction in blood was significantly associated with improved outcome in the pan cancer cohort (FIG. 13B: HR=0.96, P=0.01), this effect was again shown to be almost entirely due to blood igM/D B cell fraction that was strongly significant in the pan cancer cohort (FIG. 13B: HR=0.92, P=1.9e-6) and independently significant in colorectal adenocarcinoma (FIG. 13B: HR=0.85, P=1.5e-3). In contrast, tumour IGH B cell fraction and the blood class-switched B cell fractions failed to reach significance in the pan cancer cohort or any individual histological type.

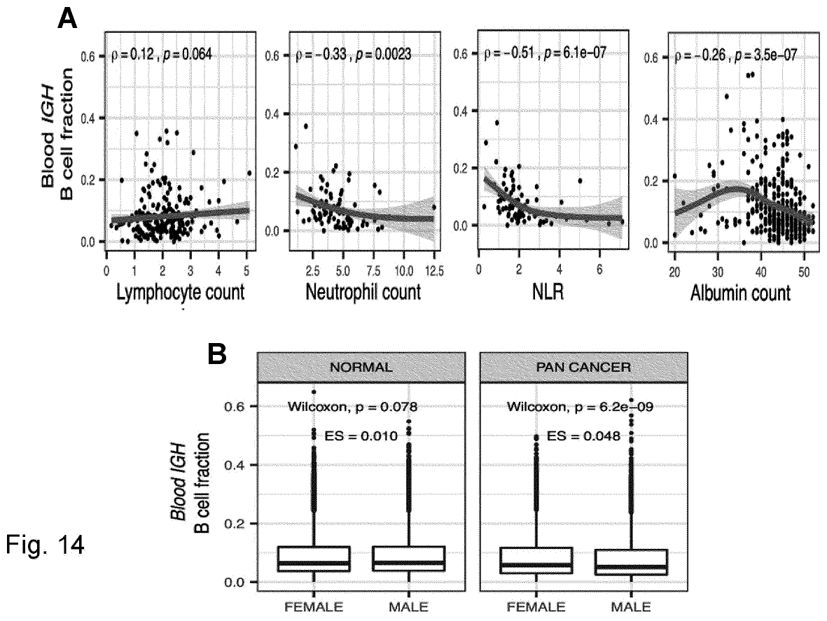

To better understand the biological basis for blood B cell fraction, the inventors correlated the scores from the present methods with date-matched blood count data within the 100KGP cohort (441 participants had blood count data from samples taken on the same date as their blood sample for WGS sequencing (see Methods). They identified a non-significant positive correlation of blood IGH B cell fraction with lymphocyte count (FIG. 14A: ρ=0.12, P=0.064), significant negative correlation with neutrophil count (FIG. 14A: rp=−0.33, P=0.0023) and strong negative association with NLR (FIG. 14A: ρ=−0.51, P=6.1e-07) as well as a negative association with albumin count (FIG. 14A: ρ=−0.26, P=3.5e-07). These data suggest that higher neutrophil counts result in a reduced fraction of B cells being measured in the WGS sample. As such, the blood B cell fraction can be viewed as a proxy for NLR and thus a signal of general systemic inflammation. Finally, the inventors examined the relationship between blood B cell fraction and biological sex. They found a significant but small sexual dimorphism compared to that observed in T cells (FIG. 14B: pan cancer ES=0.048, P=6.2e-9).

### Discussion

The advent of large-scale population sized WGS data sets such as in the 100KGP and UKbiobank has heralded a new era in genome biology. In the context of cancer this has enabled accurate measurement of the fine-details of cancer genomic abnormalities including structural variants and non-coding mutations as well as mutational signatures. In this work the inventors have fully described a new bioinformatic method, built on their previous work, to add even more value to WGS data and enable the accurate elucidation of the B cell immune compartment of a sample.

The tool uses a signal from the genomic locus undergoing V(D)J recombination in B cells to quantify B cell fraction from the IGH locus. Additionally, it predicts B cell class switching, V and J segment usage and BCR diversity. They have validated the method on the TRACERx dataset using matched data, and demonstrated that it can provide accurate B cell fraction estimates to a depth as low as 5×. The tool does not need a matched sample but can be run independently on cancer samples and matched germline blood samples.

To demonstrate the power of this tool the inventors have applied it to the 100KGP pan cancer data set where it not only provides a pan cancer landscape of immune infiltration, but the landscape of blood immune fraction.

One major advance in this updated method is the ability to accurately call B cell fraction and to deconvolve the separate class switched fractions. The inventors believe that this has much potential outside the context of cancer within autoimmunity. In particular WGS offers the ability to easily examine the B cell component from the DNA in any tissue sample and not just the circulating blood. Providing a sample can be taken this offers the ability to assess the tissue for enriched B cell content and the presence of auto-antibody producing B cells. In the context of lung cancer, the inventors found using TRACERx matched RNAseq data, that igG B cells were responsible for the majority of the B cell signal, with igM/D B cells having a much weaker signal which we hypothesis is due to the majority being in a more quiescent B cell state. This offers the possibility of distinguishing B cells that are cancer bystanders from those that have potential anti-tumour activity. In the 100KGP however the bulk of the prognostic value for the B cell fractions were not from any infiltrating B cell compartment but from the fraction of blood igM/D B cells. One hypothesis for this is that there is a benefit in having higher levels of naive B cells that are yet to encounter an antigen as all have the potential to lead to an anti-tumour response.

The primary purpose of our method is not to replace BCR sequencing but provide insight that was previously completely lacking on the BCR repertoire in samples with WGS data where any orthogonal immune-specific data is unavailable for either cost or practical reasons.

The inventors further show that the method can be adapted for use on low-pass WGS data, for example by calculating the coverage in larger bins rather than at single base resolution.

In summary, with the growing size of population-level WGS datasets, the inventors have provided a tool that can accurately quantify immune fraction without the need for additional data collection. This has allowed quantification of T and B cell content to be directly matched to genomic data that can then in turn be used to elucidate the genomic drivers of immune dysregulation.

### Example 3: WGS Based Measurement of B Cell Fraction and Class Switching Prediction with Copy Number Correction

### Introduction

In this example, the inventors developed and validated a modified version of the methods described in Example 1, in which a IGH loci specific germline and somatic copy number variant caller is used to adjust the coverage values before calculation of the B cell fraction. This model is demonstrated on a representative sample on FIG. 7D (corresponding to the sample on FIG. 7C, where FIG. 7C shows the results for the same sample without the copy number based correction) showing an example output from the method showing model fitting to the IGH locus, along with a visual representation of estimated clonotype diversity of the IGHV segment usage, as well as the percentage of predicted class-switched B cells. Comparing the data on FIGS. 7C and 7D shows that both methods lead to similar results, although in some cases with high somatic or germline copy number variations the modified version is expected to be advantageous. Extensive validation of the model was then performed by repeating some of the work in Example 2 and performing additional validations.

### Results

### Germline and Somatic IGH Focal Copy Number Correction

To quantify B cell fraction, the methods described herein utilises the IGH on chromosome 14. This locus can be subject to germline copy number variants. Therefore, to correct for both germline and somatic copy number alterations in the IGH loci before the calculation estimated IGH B cell fraction we developed both a germline and somatic IGH copy number caller.

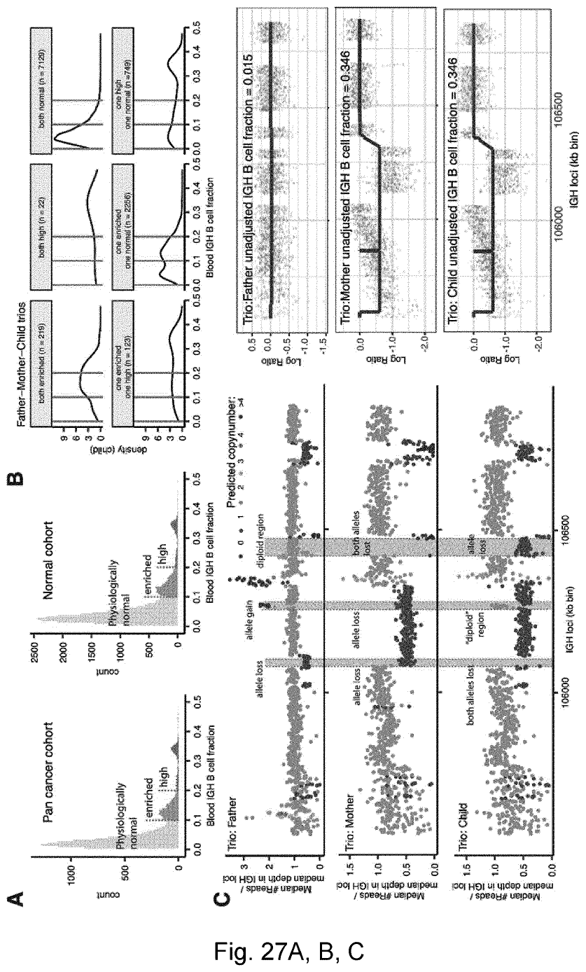

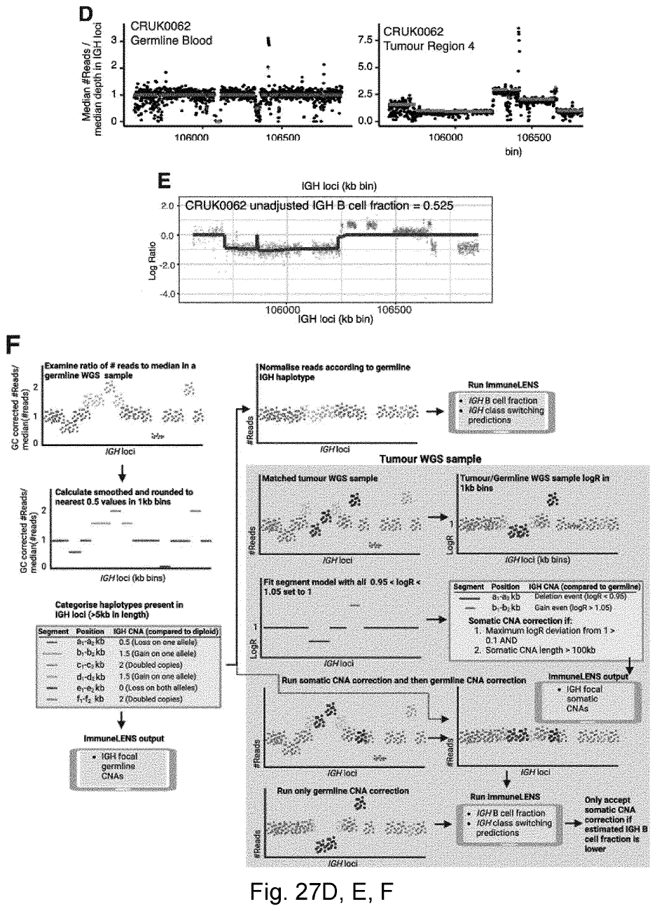

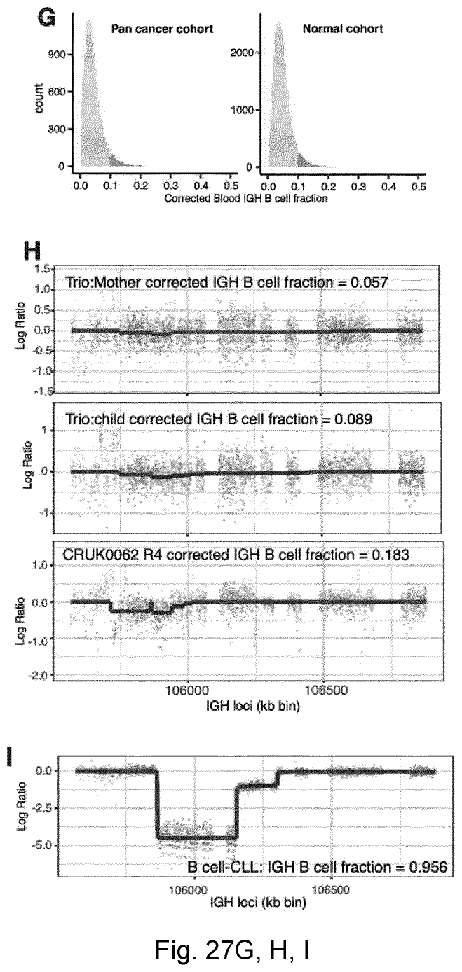

To identify the IGH loci copy number haplotype of a patient, germline blood samples were used that were assumed to have relatively low B cell content (<10%). The GC corrected coverage values were then divided by the median read depth, smoothed by taking a rolling average in 1000 bp bins and rounded to the nearest 0.5 value again in 1 kb bins. Genomic regions were then categorised into having loss, or gain events in different genomic regions. This process was then repeated 5 times, but with the GC corrected coverage values only divided by the median read depth in regions predicted to have no copy number changes at each iteration. Once the IGH germline CNVs were determined they were then used to normalise the raw IGH coverage values, with coverage values in regions of predicted loss events doubled (if only one allele lost) or removed (if deleted), and coverage values in regions with predicted gain events divided by a factor proportional to the predicted extra copies of DNA. This process is illustrated on FIG. 27F, left (in which the methods described in Example 1 are labelled as “ImmuneLENS”). Following this germline correction the method to calculate IGH B cell fraction is then used in germline samples as described in Example 1.

For somatic tumour samples the inventors first ran a simplified CNA caller within the IGH locus using paired germline and tumour coverage values with both the most focal regions of expected class switching (hg38: chr14:105712500-105860500) and V(D)J recombination (hg38: chr14:105865458-105939756) blacklisted to reduce the likelihood of class switching and V(D)J recombination events being called as tumour somatic events. For somatic CNA calling, first both the germline and tumour coverage values are adjusted for GC content and then each divided by the median value of the coverage within the IGH loci. The log R value at each base was then calculated as the log 2 ratio of the GC and total depth adjusted tumour reads to germline reads and then summarised with the median value in 1000 bp bins. The log R values are then grouped into line segments by fitting a recursive partitioning and regression tree using the rpart R package (version 4.1), for each segment the inventors calculate the median log R. The inventors then perform a breakpoint check to see if any of the somatic segment breakpoints potentially fall within the V(D)J or class switching black listed region, if they are found to, the breakpoint is adjusted to where it best fits the differences in log 2 ratio between the segments on either side of the blacklisted region. The inventors have created an automatic step to do this. For every segment that overlaps the blacklisted region, which has a log R value not equal to 1 (meaning that there is a change in copy-number that overlaps the blacklisted region) the inventors reinclude the blacklisted regions and rerun the recursive partitioning and regression tree step using the rpart package on only that overlapping segment, splitting it into subsegments. Each of these new subsegments is then checked to see if their breakpoints match the log R difference seen in the original overlapping segment with the adjacent one. If this difference is small (<20% difference between the log R change seen within the subsegments and the original adjacent segment), it is chosen as the new breakpoint. Following the calling of potential somatic CNA, it is then only used for normalisation if the segments with predicted somatic CNA are >100 kb and the maximum log R change of all the segments is >0.1. If these conditions are met, the tumour coverage regions are corrected for the somatic CNA by dividing the coverage of bases within this region by the ratio of the median coverage value within the segment to that of the baseline coverage of the median of the entire IGH locus. This process is illustrated FIG. 27F, right.

Following the somatic CNA correction, the inventors then correct for any called germline copy number alterations identified in the matched germline sample. This process is an adapted version of the germline version to take into account the effect of tumour purity and ploidy. Instead of using the theoretical normalisation values that would be valid if the tumour was diploid, for each segment representing called copy number alterations identified, the observed ratio of the coverage in that segment within the tumour sample and a baseline taken as the entire IGH locus is calculated. These normalisation values are then used directly to perform germline correction for the tumour samples. The IGH fractions are then calculated from the tumour sample using both the somatic and germline correction, depending on whether the called somatic CNA have passed the QC checks described above (somatic CNA>100 kb and maximum log R change >0.1). Even if the somatic CNA correction passed QC the germline correction is also run alone and as a final check, the somatic and germline correction version is only accepted if it is less than the germline correction alone version. This is due to the inventors' observation that samples with confirmed somatic CNAs in the IGH locus typically involve focal amplifications that lead to inflated IGH B cell fractions and the wish to be extremely conservative when calling somatic CNAs. This process is illustrated FIG. 27F, right.

For samples with predicted high B cell fraction, e.g. LCL samples, the inventors' method for calling germline CNVs was no longer valid so an adapted version was created for these cases. This followed a similar procedure as before but only used the bases at the very end (final 20 kb) of the IGH locus as a baseline for the coverage so as not to be affected by V(D)J recombination. Then dividing the locus into different regions representing the IGHV segments or class switched segments, after summarising the coverage into 1 kb bins, the inventors used the principle that coverage must be increasing along the IGHV region corresponding to a reduction in the number of V segments deleting in V(D)J recombination as you move towards the end of the IGH locus. This was done both forwards and backwards and identified potential breakpoints that are not consistent with V(D)J recombination, e.g a sudden drop and then rise in coverage as moving forward along the V segments region, which is not plausibly caused by V(D)J recombination but might represent germline CNVs such as copy number deletions. These were then categorised using the change in coverage immediately before and after the segment defined by the breakpoint in an identical manner to that used by the standard germline CNV caller.

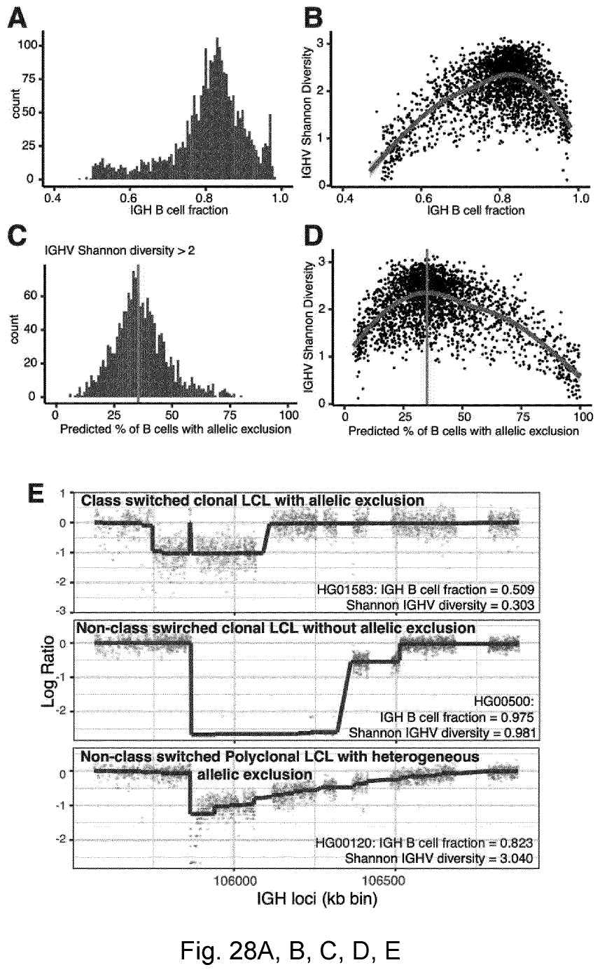

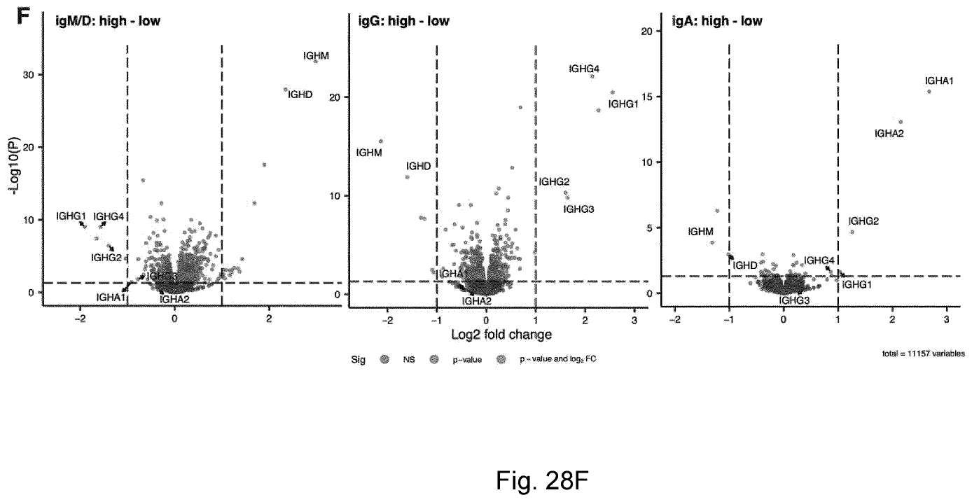

One of the complications of the IGH loci is the prevalence of germline copy number variants within the population involving deletions and duplication events (Collins et al. 2020). These variants have been recently studied in the context of disease susceptibility but in the context of estimating B cell fraction the presence of for example a large germline deletion with the IGHV loci has the potential to be misinterpreted by the present model as a V(D)J deletion event. To assess the scale of this issue with measuring B cell fraction across a large population, the inventors first applied the standard model described in Example 1 for estimating B cell fraction to the blood germline samples of the 100KGP pan cancer and rare disease cohort. In previous studies B cells have been found to make up approximately 10% of circulating lymphocytes in healthy adults (Morbach et al. 2010), however the inventors' calculations showed a substantial proportion with enriched (>10%) or high (>20%) B cell content as a percentage of the all nucleated cells (FIG. 27A). The inventors hypothesised that these high value B cell fraction samples were due to highly prevalent germline copy number variants within the human population. To test this, the inventors examined from within the 100KGP rare disease cohort all Father-Mother-Child trios. The father and mother samples were categorised into either normal physiological range, enriched (>10%) or high (>20%) and the distribution of the child's B cell fraction then examined (FIG. 27B). The inventors saw a clear signal of inheritance of high B cell fraction suggesting that these could be due to germline copy number variants. Examining individual family trios, the inventors could identify clear cases of large germline variants affecting the calculation of B cell fraction, FIG. 28C shows a case where both the mother and child have a large ~242 kb region with one allele deleted. This region is within the IGHV loci (~106082-106324 kb) and contains 19 IGHV genes used in the present model, and results in both the mother and child having a B cell fraction of 0.35 (FIG. 27C right panels).

Within the TRACERx100 cases, the inventors identified tumour samples with clear copy number changes that are inconsistent with V(D)J recombination such as in the case of tumour region 4 of CRUK0062 (FIG. 27D) resulting in less accurate IGH B cell fraction calculation.

To correct for both germline and somatic copy number alterations in the IGH loci before the calculation estimated IGH B cell fraction, the inventors developed both a germline and somatic IGH copy number caller (see FIG. 27F), as explained above. Applying this to the 100KGP germline blood sample cohort (FIG. 27G) eliminated the prevalence of large numbers of samples with either enriched or very high B cell content. The previously highlighted mother and child B cell fractions were also seen to be reduced to a normal physiological range as was the CRUK0062 R4 sample (FIG. 27H). The inventors therefore consider that germline haplotype variants within the IGH loci explain the vast majority of the calculated enriched and high IGH B cell fraction samples. An exception to this is that one 100KGP pan cancer participant was identified with extremely high predicted circulating B cell content before correction (circulating IGH B cell fraction=0.95, FIG. 27I) however this participant was identified from hospital episode statistics as having B-cell chronic lymphocytic leukemia (B-CLL) concurrently with their solid tumour that was taken for sequencing and such the germline blood taken from DNA sequencing likely comes almost entirely from these B cells.

Thus, subsequent analysis focussed on the methods using corrections for germline and somatic copy number alterations—although the methods without such corrections remain valid for all samples that do not have such IGH loci copy number alterations (as evidenced by the extensive validation in Example 2).

### Validation of WGS Based Measurement of B Cell Fraction and Class Switching Prediction

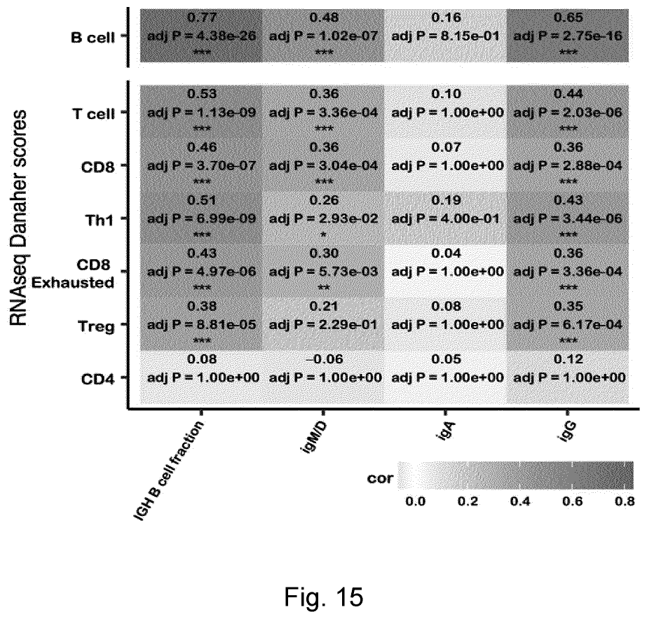

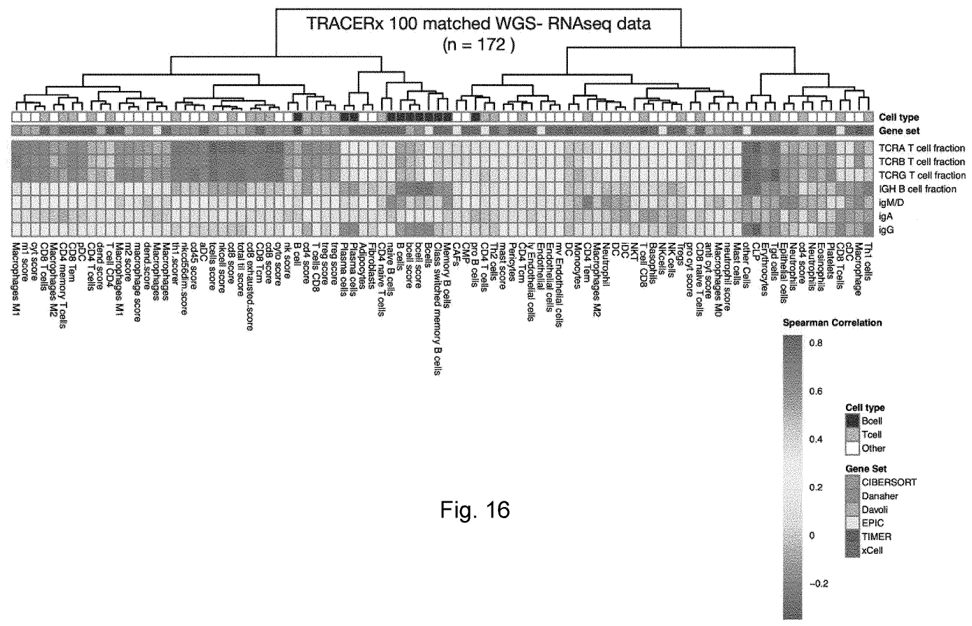

The inventors observed a strong correlation between IGH B cell fraction and the Danaher B cell score (FIG. 15) (ρ=0.77, adjusted P=4.38-26). Dividing the B cell signal by class switching status, the strongest correlations were observed with igG B cells (ρ=0.65, adjusted P=2.75-16), followed by igM/D B cells (ρ=0.48, adjusted P=1.02e-7), with no significant correlation identified with igA B cells (ρ=0.16, adjusted P=0.815). These results were also consistent with other RNA-seq based B cell related signatures produced by (Li et al., 2017), CIBERSORT (Newman et al. 2015), xCell (Newman et al. 2015) and Davoli (Davoli et al. 2017) (FIG. 16).

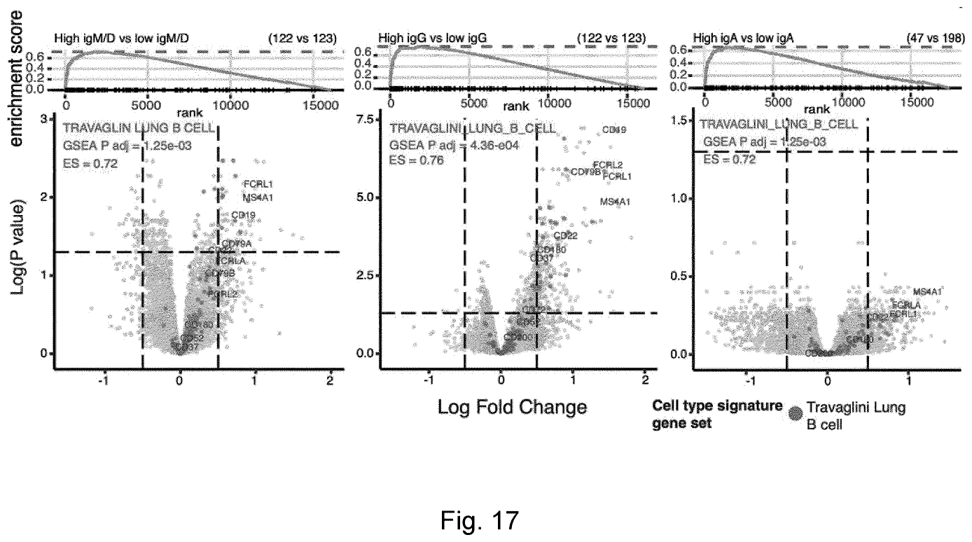

To test whether existing B cell RNAseq signatures are poorly designed to detect igA B cells, the inventors divided the TRACERx RNAseq cohort into high and low groups for igA, igG and igM/D B cells and performed a differential expression analysis (see Methods). For both igM/D and igG the inventors observed a large number of up-regulated genes in the high group samples including classical B cell markers such as CD19 and MS4A1 (FIG. 17). In contrast the igA comparison had no significantly up-regulated B cell related genes, with the caveat that there were fewer of the igA high group (n=47) due to the median fraction of igA B cells in the TRACERx cohort being 0. The inventors then performed gene set enrichment analysis on the log fold change values of these genes to identify enrichment of any cell type signature within the Molecular Signatures Database (MSigDB) C8 collection (Liberzon et al. 2011). A lung B cell gene set (Travaglini et al. 2020) was found to be significantly enriched in the high group in all three analyses (igG: adj P=4.5e-4, igM/D: adj P (=1.2e-3, igA: adj P=1.2e-3, FIG. 17).

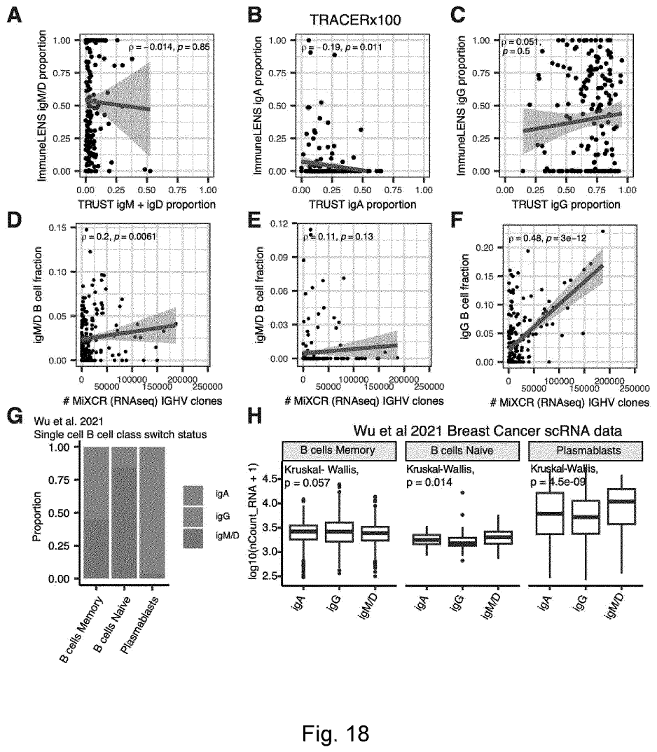

As an orthogonal method, the inventors correlated igA, igG and igM/D proportions to those calculated using the TRUST (Hu et al. 2019) method from the RNAseq data (FIG. 18A-C) as well as the number of unique IGHV clones identified from MiXCR run on the RNAseq data (FIG. 18D-F). In this the inventors only identified a significant correlation with the number of IGHV clones from MiXCR (FIG. 18F: ρ=0.48, P=3×10⁻¹²) but not the TRUST IgG calls (FIG. 18C: ρ=0.051, P=0.5) and no significant positive correlation with either TRUST or MiXCR and the igA calls.

Without wishing to be bound by theory, the inventors hypothesise that the lack of concordance with the igA B cell fraction and RNAseq data is in part due to biological differences in the level of gene expression between different class switched B cells. To test this, the inventors examined a scRNA data set (Wu et al. 2021) and categorised B cells into igA, igG or igM/D based on the expression of the IGH class switched genes. Removing the annotated B cells with unclear class switched status, the inventors identified that the majority plasmablasts were enriched for igG B cells (FIG. 18G) and that plasmablast cells had higher number of read counts than either B cell memory or naive cells (FIG. 18H), which taken together with the inventors' data suggest that igG plasmablasts may dominate any signal from RNAseq data.

In summary, the present method can produce accurate estimates of B cell content from WGS and the results show a discordance between B cell class switched predictions from DNA compared to RNA, presumably driven by a subset of the B cell compartment dominating the transcriptional signal.

### IGH B Cell Fraction Validation Using the LCL 1000 Genomes Cohort

To further validate the accuracy of the method's B cell fraction and class switching prediction, the inventors downloaded 2557 high-depth WGS (average depth=34×) files from the 1000 Genomes cohort (Byrska-Bishop et al. 2022). These samples are all derived from lymphoblastic cell lines (LCLs) which represent Epstein-Barr virus transformed B-lymphocytes typically sourced from blood samples. As expected, these samples all had an extremely high proportion of B cells (FIG. 28A). While LCLs kept in culture are expected to become clonal within 8 weeks (Ryan et al. 2006), the inventors detected many samples with high apparent diversity of B cells clonotypes with many different IGHV segments utilised in the fitted models (FIG. 28B). The observed B cell fraction was often less than 1, and a few samples had values close to 0.5. This is very likely due to allelic exclusion at the IGH locus where only one allele has undergone V(D)J recombination (Vettermann et al. 2010). The inventors' calculated B cell fractions are therefore underestimates of the true B cell fraction as they cannot account for the number of B cell clonotypes that have undergone allelic exclusion via an un-recombined and therefore non-functional IGH locus. Assuming all of the LCL samples are composed of 100% B cells, it is possible however to estimate the percentage of cells undergoing allelic exclusion. In the most polyclonal LCL samples (IGHV Shannon diversity >2) the inventors predict a median of 30% B cells have undergone allelic exclusion (FIG. 28C-D). Out of the entire 1000 genome cohort the inventors could identify samples which were either highly polyclonal or completely clonal and had differing proportions of class switched B cells (FIG. 28E).

To validate the class switching predictions the inventors downloaded the associated processed 1000 Genome transcriptomic data from the GEUVADIS study (Lappalainen et al. 2013) and ran a differential gene expression analysis between predicted B cell class switching groups. Despite the polyclonal nature of many of the samples and how DNA and RNA samples taken at different timepoints of LCL cultures may not have the same polyclonal structure, the inventors were able to identify a large number of significant genes up and down regulated after multiple hypothesis adjustment (igM/D: 808 up, 615 down; igG: 536 up, 587 down; igA: 32 up, 40 down). In particular, IGHM and IGHD had a significant enrichment in expression in samples containing high numbers of non-class switched B cells as measured from the DNA (IGHM: log FC=2.98, adj P=1.4×10⁻³², IGHD: log FC=2.35, adj P=1.1×10⁻²⁸), similarly the IGHG genes were all significantly up regulated in the igG comparison (IGHG1: log FC=2.55, adj P=5.9×10⁻²¹, IGHG2: log FC=1.61, adj P=5.1×10⁻¹¹, IGHG3: log FC=1.64, adj P=1.6×10⁻¹⁰, IGHG4: log FC=2.15, adj P=7.9×10⁻²³) as were the IGHA genes for the igA comparison (IGHA1: log FC=2.67, adj P=4.2×10⁻¹⁶, IGHA2: log FC=2.15, adj P=8.3×10⁻¹⁴) (FIG. 28F). The inventors note that the significant level of transcriptomic changes between class switched B cells (1423 genes for igM/D, 1123 genes for igG) has the potential to confound eQTL studies based on data derived from LCL cultures.

### Accurate Measurement of B Cell Fraction Persists at Low Sample Coverage

The experiments done in Example 2 to explore the minimum depth required for accurate measurement of B cell fraction using WGS data were repeated using the method after copy number correction. As above, the inventors performed nested downsampling to the TRACERx100 WGS cohort, and evaluated the ability of the present method at coverages of 60, 50, 40, 30, 20, 10, 5, 2, 1, 0.5, and finally 0.1×.

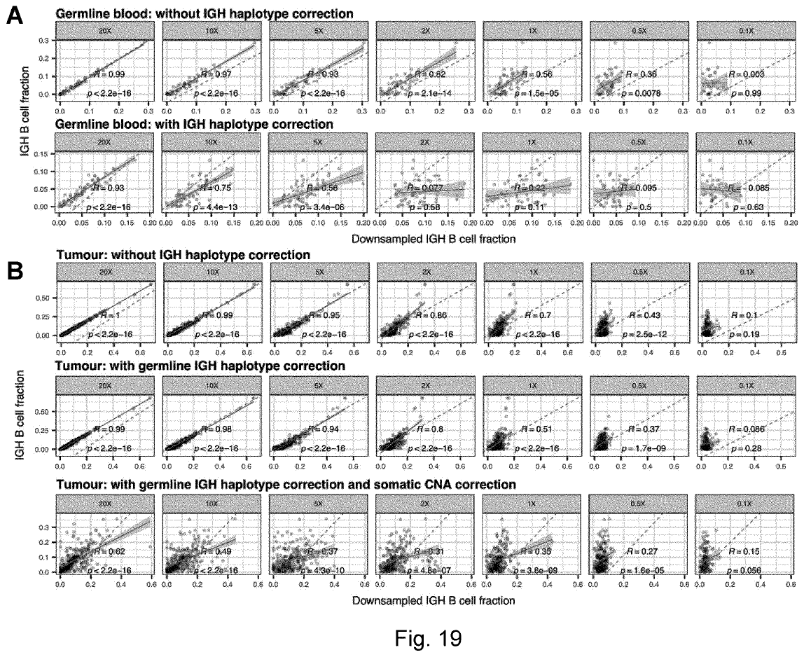

Examining specifically the germline blood samples that were downsampled from 30× to 20, 10, 5, 2, 1 and 0.5×, the inventors observed a good performance without germline haplotype correction, with accurate measurements for B cell content at depths as low as 5× at the IGH locus (FIG. 19A, 20×: R=0.99, P<2.2e-16, P<2.2e-16, 5×: R=0.93, P<2.2e-16, 1×: R=0.56, P=1.5e-5, 0.5×: R=0.36, P=0.0078) but worse performance when performing the germline copy number correction (FIG. 19A, 20×: R=0.93, P<2.2e-16, 5×: R=0.56, P=3.4e-6, 1×: R=0.22, P=0.11, 0.5×: R=0.095, P=0.5). This suggests that at least 10× is required for germline copy number inference. Similarly, somatic copy number correction was only beneficial at depths of >20×. (5×: no correction: R=0.95, P<2.2e-16; 5×: germline correction only: R=0.94, P<2.2e-16; 5× germline and somatic correction: R=0.37, P=4.3e-10, 20×R=0.62, P=2.2e-16).

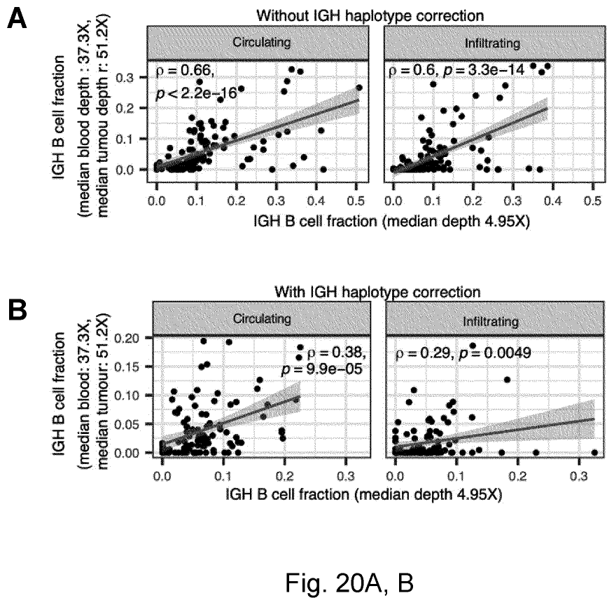

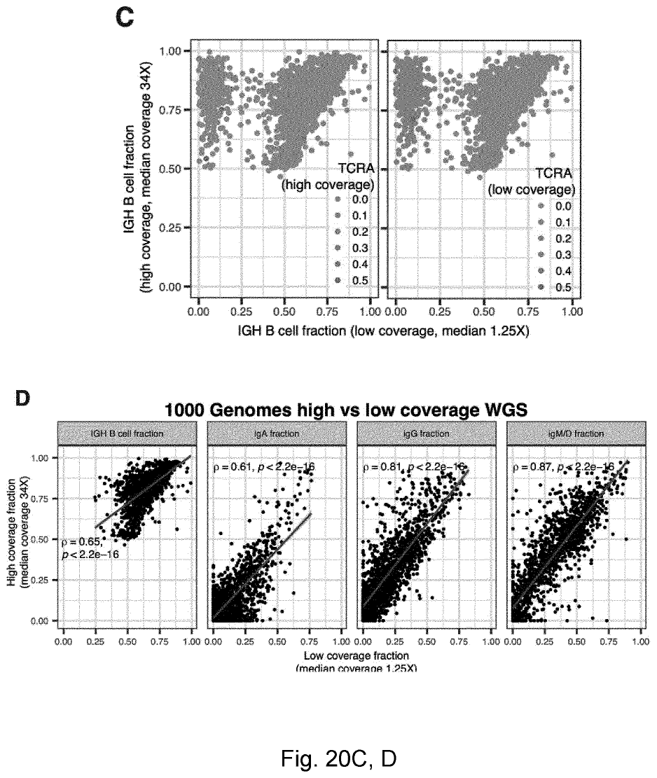

The inventors then validated these results in datasets with matched low and high depth WGS data (median depth=4.95×) samples that had also matched high depth samples that were included in the PCAWG study (blood samples median depth=37.3×, tumour samples median depth=51.2×). For IGH B cell fraction the inventors observed a much higher correlation when not using the copy number corrections (FIG. 20A) (Circulating: ρ=0.66, P<2.2e-16, Infiltrating: ρ=0.6, P=3.3e-14) than with the correction (FIG. 20B) (Circulating: ρ=0.38, P=9.9e-5, Infiltrating: ρ=0.29, P=0.0049).

The inventors also utilised the low-depth 1000 genomes samples consisting of lymphoblastoid cell lines (LCLs, median depth=1.25×) to assess the accuracy of class switching predictions. The inventors noticed that a significant number of the low-depth samples had a very low estimated B cell fraction with a high TCRA T cell fraction score (FIG. 20C), these were assumed to be newly established LCLs that had yet to be dominated by their B cell population and still contain a substantial fraction of other cell types and thus the inventors removed any sample with IGH B cell fraction <0.5 in the low-depth samples from the analysis. The inventors identified a significant correlation between high and low depth samples for IGH B cell fraction (ρ=0.65, P<2.2e-16), igA (ρ=0.61, P<2.2e-16), igG (ρ=0.81, P<2.2e-16) and igM/D B cell fraction (ρ=0.65, P<2.2e-16) (FIG. 20D).

These results show that an important factor for accurate B cell content calculation is not necessarily depth of coverage, but uniform coverage across the entire IGH locus, and this is one of the major advantages of the present method in that it is dependent on measuring a decrease in the number of aligned reads across a large genomic region rather that directly trying to align uncommon reads from very specific parts of the recombined BCR sequences.

### The Pan Cancer Immune Landscape of Circulating and Infiltrating B Cells in the 100KGP Cohort

The analysis performed in Example 2 on the 100KGP cohort were then repeated using copy number correction prior to applying the method described in Example 1. The 100KGP pan cancer cohort contains data from 14,504 participants representing 33 distinct cancer histologies with WGS samples from more than 100 participants per histology. 13,870/14,504 of these participants had a matched WGS blood sample. 634 participants including 529 with haematological cancers did not have a matched blood sample, but instead germline samples from either saliva or other normal tissue. Any tumour samples from participants that had multiple tumour samples sequenced were removed.

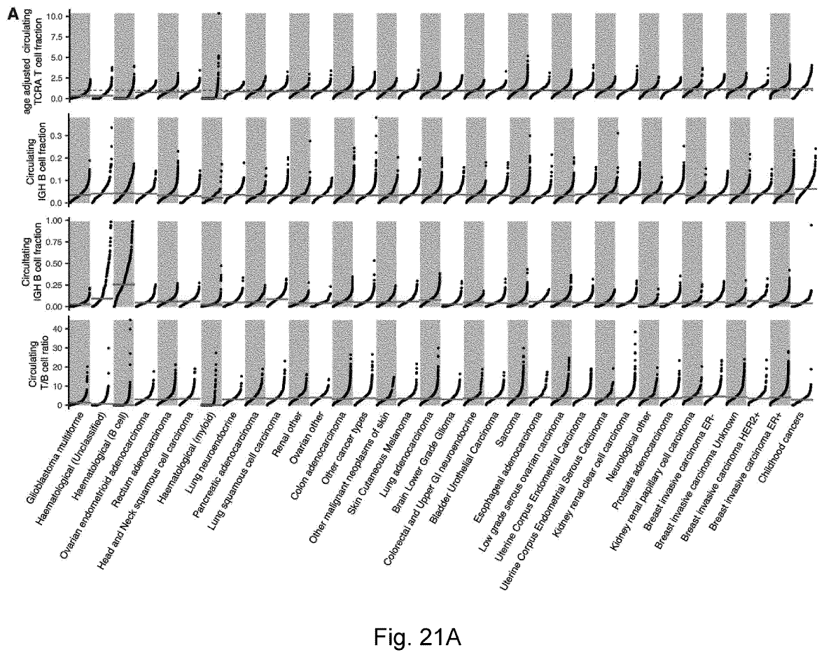

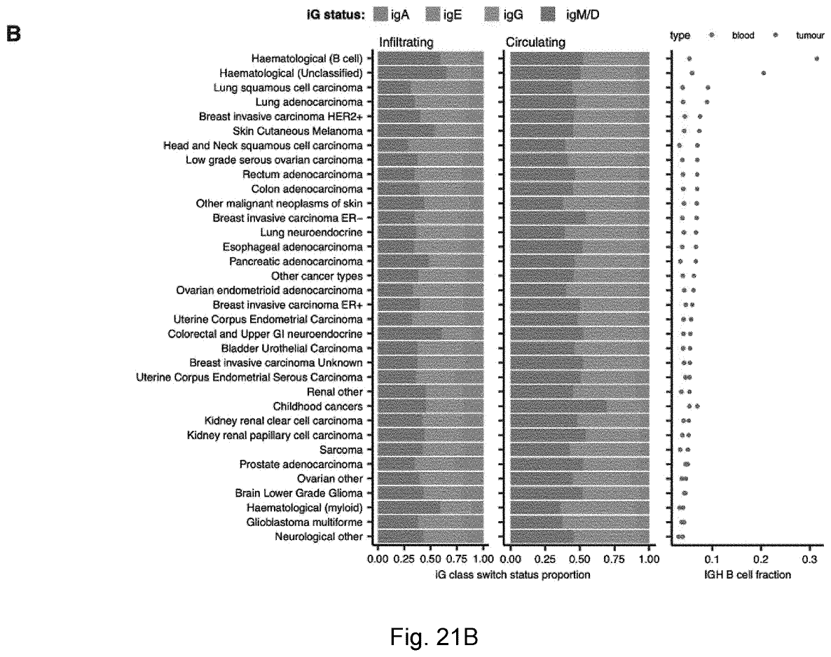

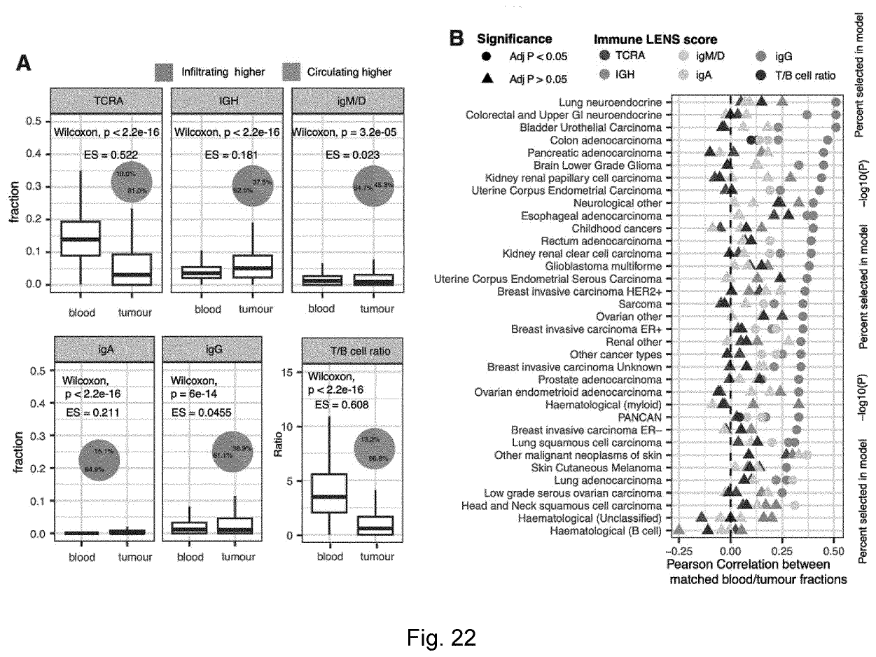

The inventors observed significant differences in the infiltrating and circulating B cell landscape across cancer types (FIG. 21A), (Kruskal-Wallis, both P<2.2×10⁻¹⁶). The present method facilitated the evaluation of differences in B cell class switching between infiltrating and circulating B cells in different cancer types. The inventors observed that infiltrating B cells were present at a higher fraction than circulating B cells (ES=0.214, P<2.2×10⁻¹⁶, 65% participants higher infiltrating, FIG. 22A), this is in contrast to infiltrating T cells which were present at higher fractions than circulating T cells (ES=0.522, P<2.2×10⁻¹⁶, 81% participants higher circulating, FIG. 22A). The elevated infiltrating B cell levels compared to circulating was primarily driven by enrichment in IgA and IgG B cells (IgA: ES=0.222, P<2.2×10⁻¹⁶, 85.2% participants higher infiltrating; IgG: ES=0.11, P<2.2×10⁻¹⁶, 68.1% participants higher infiltrating, FIG. 22A), a much smaller but significant difference was observed in non-class switched B cells (igM/D: ES=0.0542, 52.2%, P<2.2×10⁻¹⁶ participants higher in tumour, FIG. 22A). This highlights key differences in the function and make-up of circulating and tumour infiltrating B cells. Consistent with this, the inventors observed a significantly higher T/B cell ratio in blood compared to tumour samples (ES=0.623, P<2.2×10⁻¹⁶, FIG. 22A).

The correlation between circulating and infiltrating IGH B cell fraction was strongly significant in all histologies, driven predominantly by a very strong correlation in the IgA B cell fraction (Pan cancer: R=0.75, adj P<2.2×10⁻¹⁶, FIG. 22B).

Recent studies have focused on the immune landscape of the tumour microenvironment within a pan cancer setting (Thorsson et al. 2019), instead due to the availability of estimated B cell fractions in the 100KGP germline blood samples the inventors are able to perform an in depth investigation of the immune landscape in the peripheral blood of cancer patients.

### Circulating TCRA T Cell Fraction is a Proxy for Neutrophil to Lymphocyte Ratio (NLR)

The inventors applied the present method to the entire 100KGP cohort of both cancer and rare disease participants resulting in calculated immune fraction scores from 91,084 WGS bam files. Of these, 62,079 bam files are derived from normal tissue samples taken from participants within the 100KGP rare disease cohort containing samples from both probands and non-affected relatives. The remaining bam files consist of matched tumour and germline samples taken from participants in the 100KGP pan cancer cohort. The vast majority of the germline samples (13870/14504 of cancer germline and 60758/62079 of rare disease cohort) are derived from blood samples, and hence the calculated lymphocyte fractions represent circulating T or B cells at the time of sampling.

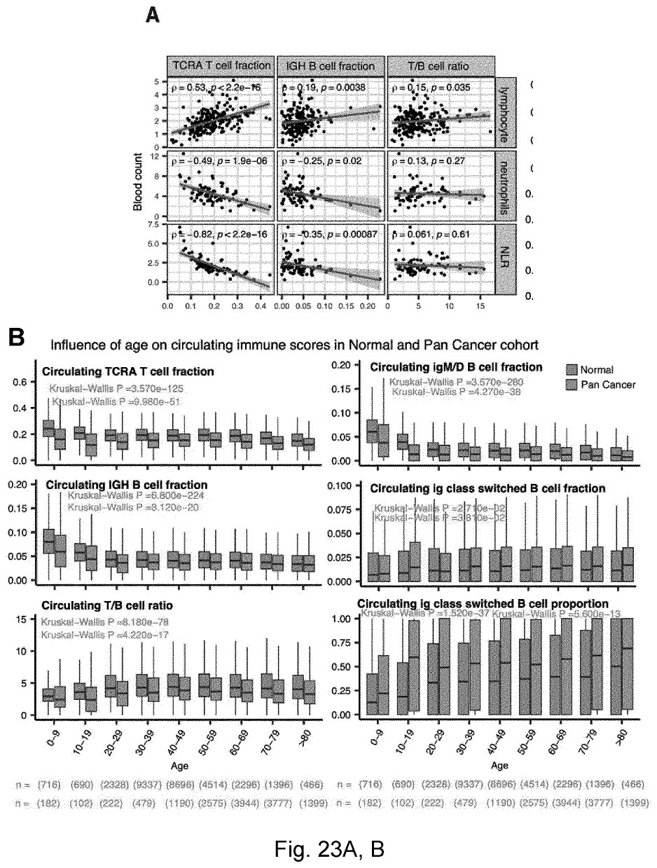

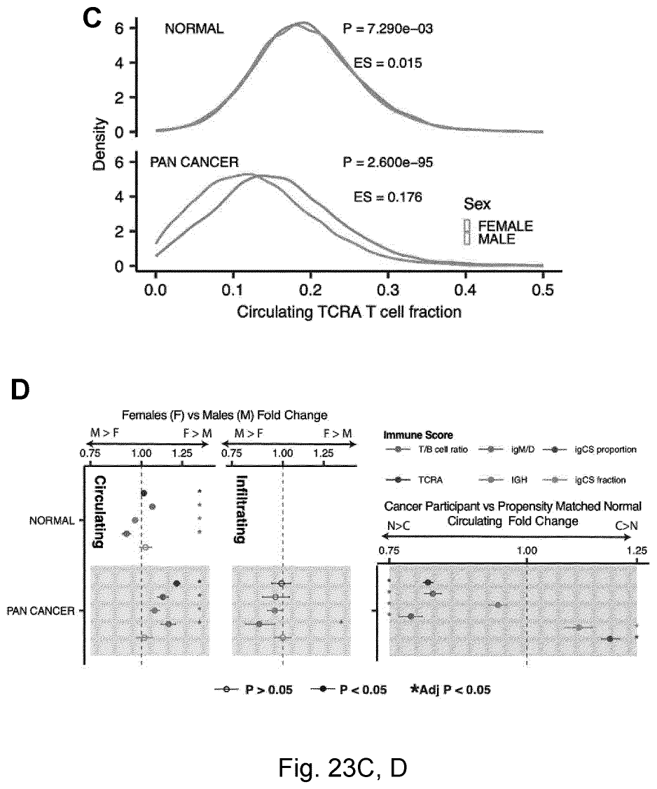

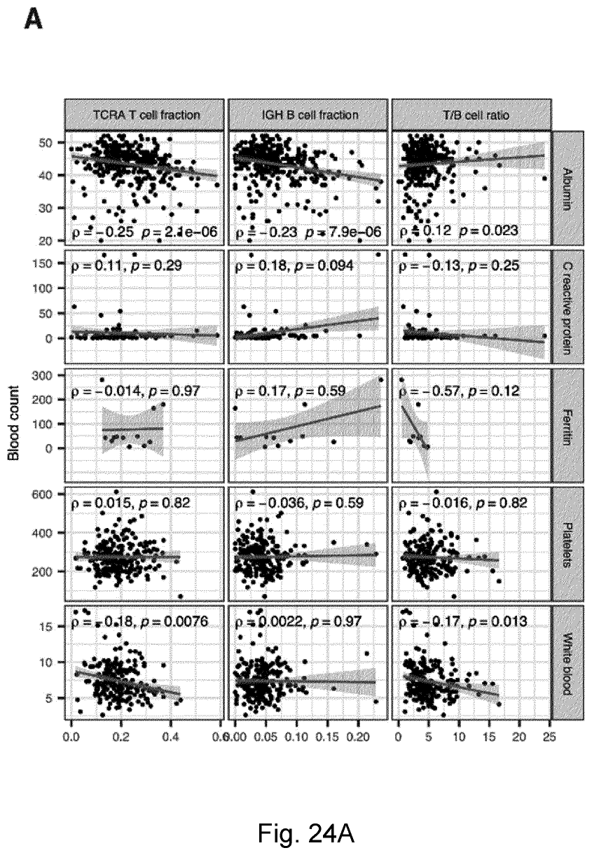

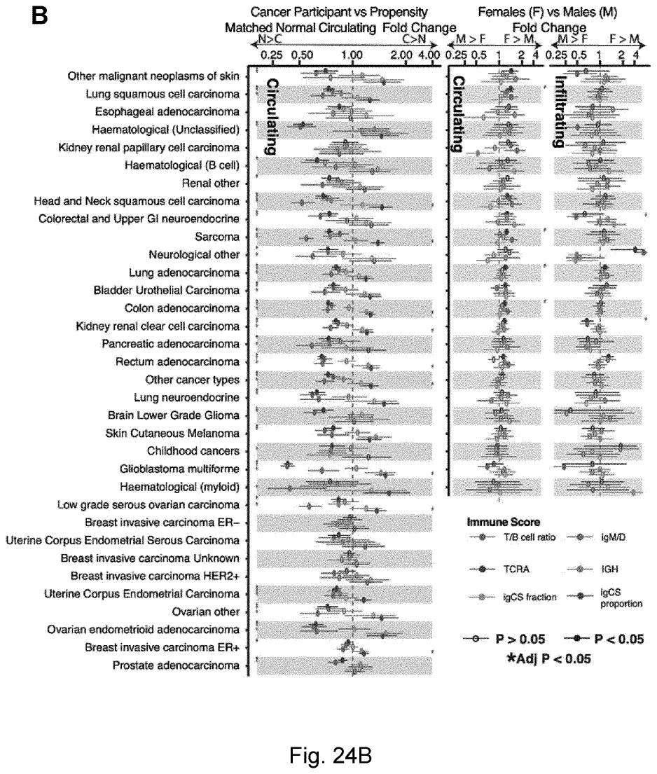

To better understand the biological basis for circulating TCRA T cell fraction, the inventors correlated calculated scores with date-matched blood count data within the 100KGP cohort (441 participants had blood count data from samples taken on the same date as their blood sample for WGS sequencing, see Methods). The inventors found a significant positive correlation between circulating T cell fraction and lymphocyte count as expected (FIG. 23A: ρ=0.53, P<2.2×10⁻¹⁶) but also a significant negative correlation with neutrophil count (FIG. 23A: ρ=−0.49, P=1.9×10⁻⁶) and a strong association with the neutrophil to lymphocyte ratio (NLR) (FIG. 23A: ρ=−0.82, P<2.2×10⁻¹⁶). These data suggest that higher neutrophil counts result in a reduced fraction of T cells being measured in the WGS sample. As such, the circulating TCRA T cell fraction can be viewed as a proxy for NLR and thus a signal of general systemic inflammation. In addition, the inventors found a significant but weak negative correlation between blood T cell fraction and albumin concentration (FIG. 24A: ρ=−0.25, P=2.1×10⁻⁶), a separate signal for general inflammation. We identified similar trends for circulating IGH B cell fraction with a non-significant positive correlation with lymphocyte count (FIG. 24A: ρ=0.12, P=0.064), significant negative correlation with neutrophil count (FIG. 24A: ρ=−0.33, P=0.0023) and strong negative association with NLR (FIG. 24A: ρ=−0.51, P=6.1×10⁻⁷) as well as a negative association with albumin count (FIG. 24A: ρ=−0.26, P=3.5×10⁻⁷). No significant associations were found with other blood count measures such as C reactive protein and ferritin (FIG. 24A), apart from a weakly negative association between white blood cell count and TCRA T cell fraction (FIG. 24A: ρ=−0.18, P=0.0076) and T/B cell ratio (FIG. 24A: ρ=−0.17, P=0.013).

To obtain a measure of circulating lymphocyte cell content within the blood independent of neutrophil levels the inventors also developed a metric based on the ratio of T cell to B cell fraction, or the T/B cell ratio. Since both of the circulating T and B cell fractions calculated are dependent on the neutrophil proportion of the blood, their ratio is independent of neutrophil content (see Methods). In the matched blood count data the inventors found that the T/B cell ratio was significantly associated with lymphocyte count (FIG. 23A, ρ=0.15, P=0.032) but not neutrophil count (FIG. 23A, ρ=0.029, P=0.81) or NLR (FIG. 23A, ρ=0.043, P=0.73).

### Determinants of Circulating Leukocyte Fractions in Cancer Patients

The importance of circulating leukocyte and other measured blood counts has been widely acknowledged in cancer (Templeton et al. 2014). The 100KGP data set allows a systematic investigation of the dysregulation of blood counts across multiple cancer types and how it compares to a cohort of normal participants taken from the relatives within the rare disease arm of the 100KGP data.

The inventors first sought to examine the influence of age on immune infiltrate. Dividing the 100KGP participants in age deciles, consistent trends were observed in both the normal and pan cancer cohort of the levels of both T and B cell fraction declining with age. For B cells this effect was strongest for the non-class switched B cells, with the proportion of class switched B cells increasing with age (FIG. 23A). It is notable that across every age decile, circulating TCRA T cell fraction and IGH B cell fraction was decreased in cancer participants, while the percentage of class switched B cells was increased.

Due to the well-established sexual dimorphism of the immune system (Klein et al. 2016) the inventors next examined the relationship between circulating lymphocyte count and biological sex. The inventors first noted a significant and large decrease in blood T cell fraction in males compared to females in the pan cancer cohort (FIG. 23B: ES=0.173, P<2.2×10⁻¹⁶) but only a small, albeit significant, change across normal 100KGP participants (FIG. 23B: ES=0.017, P=0.0031). The inventors quantified the fold change (FC) of all measured lymphocyte counts between females and males using a bootstrap method to obtain a 95% confidence interval. For the entire pan cancer the circulating TCRA T cell fraction was 21% (log P=−161) higher in females than males, with the IGH B cell fraction being 6.9% (log P=−16) higher, driven entirely by the igM/D subpopulation (15% higher, log P=−22) with the class switched B cells being non-significant. This was in contrast to the normal cohort where total IGH and igM/D B cells were decreased in females compared to males (IGH: 3% decrease, log P=−12, igM/D: 7.3% decrease, log P=−30) and only a small increase in TCRA T cell fraction (1.2% increase, log P=−6) (FIG. 23C). While differing neutrophil levels may explain some of the effect seen in the cancer cohort, the inventors also saw the T/B cell ratio higher in females (Pan cancer: 11% higher, log P=−31).

Due to the widespread sexual dimorphism in circulating TCRA T cell fraction observed specifically in patients with cancer as well as the differences across age deciles, the inventors looked more generally at the differences between circulating lymphocyte fraction in the healthy versus cancer context. Using propensity matching to select matching cohorts with the same age and sex distribution for each disease type, the inventors found that T cell fraction along with IGH B cell fraction and igM/D B cells was significantly decreased in the pan cancer setting (TCRA: 18% decrease, log P=−786, IGH: 5.3% decrease, log P=−22, igM/D: 21% decrease, log P=−195) while class switched B cells in fraction and proportion of total B cells were increased (10.5% increase) (FIG. 23C, right panels). It is notable that for both the observed sexually dimorphism and general differences between cancer and normal participants that the T/B cell ratio was also significant (17% decrease in cancer compared to normal, log P=−242) and thus these differences can not be solely explained by altered neutrophil levels.

Looking at these effects across the different cancer histologies (FIG. 24B), the inventors observed higher circulating TCRA T cell fractions in females (FC>1) with P<0.05 in 12 different cancer histologies, though this was only significant after multiple hypothesis testing in lung squamous carcinoma (FC=1.34, adjusted P=2.0×10⁻⁵), sarcoma (FC=1.19, adjusted P=2.0×10⁻⁴), lung adenocarcinoma (FC=1.16, adjusted P=4.5×10⁻²) and colon adenocarcinoma (FC=1.14, adjusted P=2.0×10⁻⁵). In contrast, looking at sexual dimorphism in infiltrating lymphocytes, the inventors observe little effect with overall igM/D levels in pan cancer being reduced in females (FC=0.88, log P=−7.3, adj P=0.15), and a few cancer type specific effects such as kidney renal cell carcinoma having higher TCRA T cell fraction in males than females (28.7% higher, adjusted P=0.0016), which is consistent with the immune sexual dimorphism observed from transcriptional analysis of renal cancer (Laskar et al. 2021). These data suggest that while biological sex has a significant impact on the levels of circulating lymphocyte cells in blood of cancer patients, associations of sex and lymphocyte infiltrate in tumours is restricted to lung adenocarcinomas and renal cancers, and only weak associations are observed with the circulating T cell content of healthy individuals.

Looking at the propensity matched analysis split by cancer type, the greatest difference in circulating T cell fraction between cancer patients compared to healthy controls was seen in patients with glioblastoma multiforma (FIG. 24B, FC=0.38, adjusted P=4.4×10⁻¹⁶⁹), and the least in patients with breast invasive carcinoma (FIG. 24B, FC 0.97, adjusted P=1), the reduction was pervasive across cancer histology with T cell fraction being significantly lower in 26/34 different histologies after accounting for multiple hypothesis testing. Given that T cell fraction reflects the proportion of T cells in the sequenced sample, this reduction could be due to a reduced T cell count in the blood of cancer patients or a relative increase of other cell types, such as neutrophils. Increased peripheral blood neutrophil count, or neutrophilia, is common across many cancers (Grecian et al. 2018) and some cancers such a glioblastoma are frequently treated with steroids that are known to increase neutrophil count (Ronchetti et al. 2018). However, the T/B cell ratio which is independent of neutrophil count is still significantly lower in 19 different cancer histologies including glioblastoma multiforma (FIG. 24B, FC=0.38, P=4.4×10⁻⁷²) after accounting for multiple hypothesis testing.

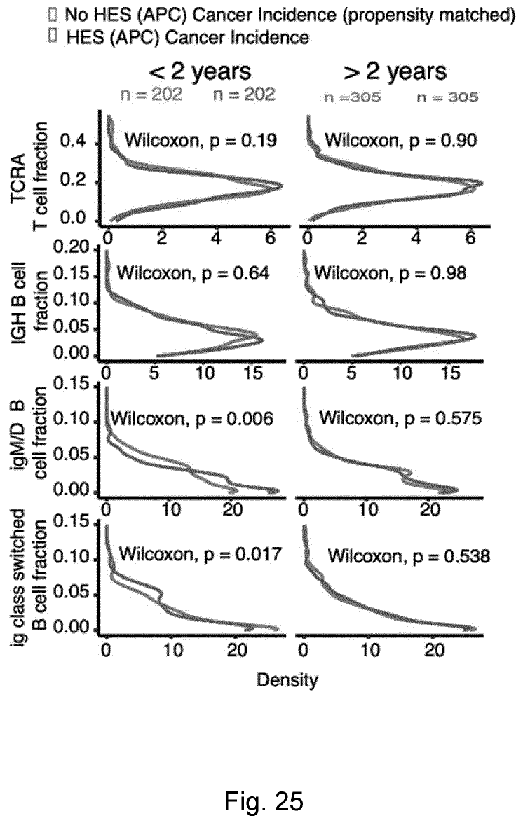

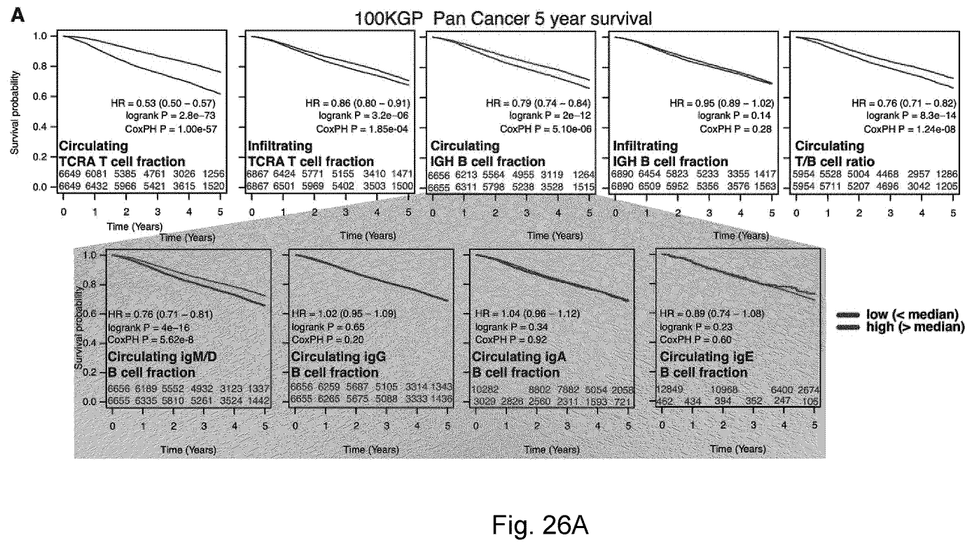

To evaluate whether the low levels of immune infiltrate observed in patients with cancer can be detected prior to cancer diagnosis, the inventors identified within the 100KGP normal cohort 507 participants with no previous history of cancer prior to DNA sequencing of their germline blood who later were treated for cancer according to NHS hospital episode statistics. The cohort was then divided into those who received cancer treatment <2 years after DNA sequencing and those who received in >2 years, and tested for differences in our estimated lymphocyte fractions compared to a propensity matched for age and sex who did not receive any treatment for cancer of the same size. The inventors saw no significant difference for the cohort that received cancer treatment >2 years after treatment, but within the cohort who received treatment <2 years there was a significant reduction for igMD B cells (FIG. 25, P=0.0022) and an increase in class switched B cells (FIG. 25, P=0.012) in those participants that went on to receive a cancer diagnosis. No significant difference was observed for T cell fraction. This result could be interpreted as either the effect of early stage non-diagnosed cancer on the circulating B cell fractions similar to that seen in diagnosed cancer or participants who go on to get cancer have a greater ‘immunological age’ and therefore more predisposed to develop cancer.

Circulating Lymphocyte Cells are More Prognostic of Survival than Tumour Infiltrating Lymphocyte Cells

The presence and quantity of infiltrating tumour lymphocyte cells have been previously shown to be prognostic for survival in many cancer types (Bentham et al. 2021, Nosho et al. 2010, Hwang et al. 2012). Similarly, circulating immune cell data from blood counts have been demonstrated to have prognostic importance (Ying et al. 2014, Kumarasamy et al. 2019). Using the present method to measure both the circulating and tumour infiltrating T and B cell fraction from WGS simultaneously at the time of sample collection, the inventors were able to directly compare the prognostic value of both measurements in a pan cancer setting.

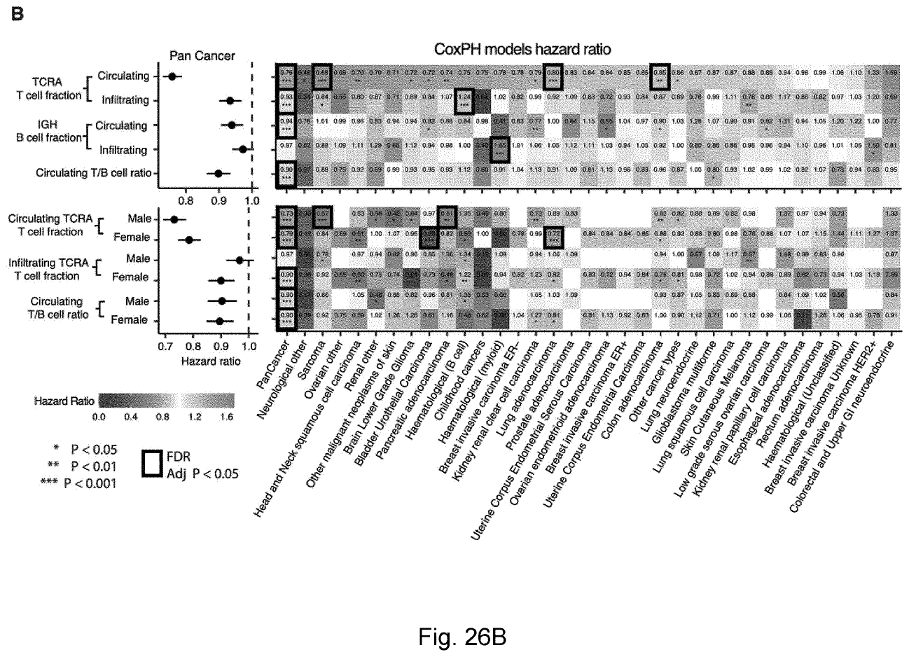

Across the pan cancer cohort, elevated circulating TCRA T cell fractions were significantly associated with improved overall survival outcomes (FIG. 26A, HR=0.54, logrank P=4.1×10⁻⁷³, high and low groups split by the median). Higher infiltrating TCRA T cell fraction was also associated with improved survival, however the association was less pronounced (FIG. 26A, HR=0.86, logrank P=3.1×10⁻⁶); the hazard ratio associated with elevated blood TCRA T cell fraction was significantly smaller (P value of 2.59×10⁻²³, Student t test on the beta coefficients from the two Cox models).

The IGH B cell fraction also followed the same trend, with the high fraction group being more significant for circulating B cells (FIG. 26A, HR=0.82, logrank P=1.3×10⁻⁸) than infiltrating where it did not reach significance (FIG. 26A, HR=1.02, logrank P=0.6). While the effect of circulating T cell fraction may be partly driven by neutrophils, the inventors also found the circulating T/B cell ratio significantly associated with prognosis (FIG. 26A, HR=0.76, logrank P=8.3e-14). Splitting up IGH B cell fraction by the class switched fractions revealed that patients with high non-class switched circulating igM/D B cell fraction were associated with improved prognosis (FIG. 26A: igM/D: HR=0.79, logrank P=4.6×10⁻¹³), while blood IgG, IgA or IgE B cell fraction was not associated with a significant survival difference.

To explore the relationship of lymphocyte cell fraction with other clinical features and their heterogeneity across different cancer types, Cox proportional hazard (CoxPH) models were fitted, controlling for age, sex, stage and whether the patient received chemotherapy pre-surgery and sample collection. Circulating TCRA T cell fraction in blood was highly significant in the pan cancer cohort (FIG. 26B: HR=0.76, P=2.6×10⁻⁴⁶), as well as three individual cancer types after multiple hypothesis correction (FDR method): colon adenocarcinoma (HR=0.84, adj P=2.5×10⁻²), lung adenocarcinoma (HR=0.80, adj P=1.1×10⁻²) and sarcoma (HR=0.68, adj P=5.2×10⁻⁴). In contrast, increased tumour infiltrating TCRA T cell fraction, was only significant after multiple hypothesis adjustment (FDR) in the pan cancer cohort (HR=0.93, adj P=8.7×10⁻³), and B cell haematological cancers (HR=1.24, adj P=8.7×10⁻³) where it was associated with worse prognosis. Circulating T/B cell ratio was only significant at the pan cancer level after multiple hypothesis adjustment (HR=0.90, adj P=8.6×10⁻⁶), suggesting that both circulating T cells and neutrophils contribute to the prognostic value of circulating TCRA T cell fraction.

We next investigated factors that may influence the prognostic associations identified above. Considering the sexual dimorphism observed in circulating T cell fraction within cancer patients, we first ran CoxPH models stratified by sex (FIG. 26B). In the pan cancer context, males and females exhibited a similar association between high blood TCRA T cell fraction and improved prognosis (Males: HR=0.73, P=1.5×10⁻²⁷; Females: HR=0.79, P=2.6×10⁻²⁰). However, stratifying by sex also revealed cancer type specific differences. After multiple hypothesis adjustment, higher circulating T cell fraction was significantly associated with improved prognosis for females in bladder urothelial carcinoma (HR=0.28, adj P=5.4×10⁻³) and lung adenocarcinoma (HR=0.72 adj P=5.4×10⁻³). For males however it was significantly associated with improved prognosis in sarcoma (HR=0.56, adj P=7.7×10⁻⁵) and pancreatic adenocarcinoma (HR=0.61, adj P=4.0×10⁻²). To quantify the significance of sexual dimorphism in terms of prognostic value, the inventors added an interaction term between sex and circulating TCRA T cell fraction to the CoxPH model. This was significant in bladder cancer (HR=3.16, P=0.00088) but no other solid cancer types.

The prognostic value of circulating TCRA T cell fraction likely reflects a combination of two factors: 1) a signal related to systemic inflammation which is correlated to the circulating T cell fraction but not due to the function of the circulating T cells themselves, 2) a signal reflecting the ability of circulating T cells to recognise neoantigens and therefore prevent metastasis, cancer spread and improve patient outcome. Considered together, these results suggest that circulating lymphocytes sampled at the time of surgery represent a more predictive biomarker for cancer patient prognosis than infiltrating lymphocyte cells.

### Discussion

Building on the method provided in Example 1, and validated in Example 2, the inventors further improved the method of IGH B cell fraction quantification, by incorporating an IGH loci specific germline copy number variant caller. The variant caller is used to adjust the coverage values before calculation of B cell fraction, as described above. The improved method was again extensively validated using matched WES, WGS and RNA-seq samples from the TRACERx100, 100KGP pan cancer, and 1000 Genome cohort dataset.

Improved accuracy in IGH B cell fraction quantification is dependent on accurate quantification of potential germline copy number alterations. These have not been extensively characterised in large populations. The method presented in Example 3 corrects the predicted B cell fraction based on the calculated germline diversity of the IGH loci.

### Methods

### Statistics

All statistical tests were performed in R 3.6.1 (Example 2) or R 4.0.2. (Example 3). No statistical methods were used to predetermine sample size. Tests involving correlations were done using stat_cor from the R package ggpubr (v0.4.0 in Example 2, v0.6.0 in Example 3) with the Spearman's method. Tests involving comparisons of distributions were done using stat_compare_means using wilcox.test using either the unpaired option, performing a Wilcoxon rank-sum (Mann-Whitney U) test, or a paired Wilcoxon signed-rank test. Effect sizes for the corresponding Wilcoxon tests were measured using the wilcox_effsize function from the rstatix package (v0.6.0 in Example 2, v0.7.2 in Example 3). Hazard ratios and P values were calculated with the survival package (v3.2-3) for both Kaplan-Meier curves and the Cox proportional hazard model. Hazard ratios between different models were compared with the hr.comp2 function from the survcomp package (v1.40.0). For all statistical tests, the number of data points included are plotted or annotated in the corresponding figure. Plotting and analysis in R also made use of the ggplot2 (v3.3.3 in Example 2, v3.4.1 in Example 3), dplyr (v1.0.4 in Example 2, v. v1.1.0 in Example 3), tidyr (v1.1.1 in Example 2, b1.3.0 in Example 3), gridExtra (v2.3) and gtable (v0.3.0) packages. P value adjustments were made using the Holm-Bonferroni method unless stated otherwise.

### TRACERx100

The first 100 patients prospectively analysed by the NSCLC TRACERx study (clinicaltrials.gov/ct2/show/NCT01888601, approved by an independent research ethics committee, 13/LO/1546) were used in this study. This is identical to the 100-patient cohort originally described in Jamal-Hanjani et al. (2017). In brief, informed consent was a mandatory requirement for entry into the TRACERx study. This NSCLC cohort consisted of 68 males and 32 females with a median age of 68. Finally, the cohort was predominantly made up of early-stage tumours (Ia (26), Ib (36), IIa (13), IIb (11), IIIa (13) and IIIb (1)) and 28 patients also had adjuvant therapy.

Both WES (aligned to the hg19 sequence) and RNA-seq samples were obtained from the TRACERx study for the first 100 patients; the method for processing these samples is as previously described13. Notably, for the WES samples, exome capture was performed using a custom version of Agilent Human All Exome V5 kit according to the manufacturer's instructions.

TRACERx100 samples were sequenced and aligned to GRCh38 by Genomics England using the same Illumina sequencing pipeline as used in the 100KGP to produce WGS samples. WGS coverage values from the IGH loci for these samples were then extracted using samtools depth. No other WGS derived data besides these coverage values was utilised from the TRACERx cohort for this analysis.

Calculation of TRACERx100 BCR Metrics from RNAseq Data

MiXCR (Bolotin et al., 2015) was used to call BCR clonotypes from TRACERx100 RNAseq data directly, from these calls the Shannon entropy was then calculated using the proportions of the BCR clonotypes found in each sample.

TRUST4 (Song et al. 2021) was also used to call BCR sequences and therefore infer class switching proportions.

### Differential Gene Expression Analysis and Gene Set Enrichment

Differential gene-expression analysis was performed on the TRACERx100 patients with RNA-seq data separating the cohort into high or low groups based on either IGH B cell fraction or class switching B cell fraction scores for IGHG1, IGHA1 B cell fraction or non-class switched igM/D B cell fraction. First, using R 4.0.0, the edgeR package (version 3.32.1) was used for sample-specific trimmed mean of the M-values (TMM) normalization; any genes with low expression were then filtered out using the standard edgeR filtering method before using the Limma-Voom method from the limma R package (version 3.46.0) to calculate the Voom fit and obtain P-values for the gene-expression differences. The comparison controlled for patient and histology as blocking factors and P-values were FDR-corrected for multiple testing. Results were then visualised with the R EnhancedVolcano package (version 1.8.0). Gene set enrichment analysis was then performed using using the fgsea R package (version 1.24.0) using the MSigDB C8 gene sets of cell type signatures (www.gsea-msigdb.org/gsea/msigdb/human/genesets.jsp?collection=C8).

### 100KGP WGS Cohort

T and B cells were calculated for the entire 100KGP cohort (lung: data release v8 (2019 Nov. 28), remaining pan-cancer data release v12 (2021 May 6), and rare disease v12 (2021 May 7). In total, scores were calculated for 92,905 WGS bam files.

Of these 31,675 bam files were part of the 100KGP cancer cohort, representing 16,294 cancer bam files and 15,381 germline bam files. Some participants had multiple tumour samples collected for WGS, due to lack of annotation of the reason for multiple samples (e.g. technical resequencing, representative of metastasis, multiple region sequenced or occurrence of a second primary tumour at a later time point) these were removed from the pan cancer cohort leading to a final cohort of 14,576 tumour WGS samples. The germline samples were restricted to those taken from matched germline blood samples representing 14,664 WGS samples. Of the 14,576 tumour WGS samples, 13,941 have a matched blood WGS sample.

For the rare disease cohort scores for 61,230 bams in total were calculated. Limiting to samples taken from blood samples leads to 59,912 bams representing 29,238 samples taken from probands with a rare disease and 30,665 relatives of these probands. This cohort of 30,665 blood samples from relatives was taken as our normal cohort to compare with the 14,664 blood samples from cancer patients. Additionally, from this normal cohort propensity matched cohorts for each cancer histology were created using the R package matchit controlling for both age and sex.

B cell fractions were calculated with the WGS version of the method described above, adjustments for tumour purity were made using estimates from Genomics England and local copy number using CANVAS (version 1.3.1) (Roller et al. 2016) calls produced by Genomics England for nearby genes to the V(D)J loci (IGH: TMEM121).

Cancer histology in terms of disease and disease subtype was curated by Genomics England and is as described in the cancer_analysis_table available to researchers within the Genomics England research environment, with disease_subtype being set to ‘OTHER’ in cases with occurrence less than N cases in the total cohort, for both ease of analysis and to avoid using any identifying features in the analysis.

### Nested Down-Sampling of WGS Files

Nested downsampling was performed on WGS bam files using samtools view (version 1.14) recursively with the following options: samtools view -b -h --subsample FRAC - -subsample-seed SEED BAM CHR_LOC>OUT_BAM

Depth of original bam files were calculated with mosdepth (version 0.3.2) and then downsampled to 60×. After each downsampling each output bam was indexed using Picard (version 2.20.3) BuildBamlndex before being downsampled again with samtools view to create a set of nested downsampled bams with depths of 60, 50, 40, 30, 20, 10, 5, 2, 1, 0.5 and 0.1×. With the FRAC values being calculated to obtain these depths based on the depth of the original bam file as calculated with mosdepth. SEED values were changed in each nested down sample to avoid using the same seed repeatedly.

The above procedure was done to generate downsampled bams for all of the TRACERx WGS samples for the IGH (chr14:105566277-106879844) locus. These downsampled bams were then used as input for the WGS version of the methods described herein to calculate the B cell fraction.

### Analysis of WGS Data Using a Segment-Based Model

All results in this example were obtained using WGS data and the segment-based model described in Example 1. As explained above, this models the read depth ratio as a series of constant piecewise segments. Here the segments are pre-chosen and align with the locations of the V and J segments. Additionally, we know from V(D)J recombination that the read depth ratio should start from 0 and from there must be monotonically decreasing until the point of maximum V(D)J recombination. For example in TCRA V(D)J recombination only some TCR chains will have selected the first TRAV-1 segment and all other V segments will be deleted, however all TCR chains will have a deletion following the final V segment. Likewise for J segments these must be monotonically increasing until reaching 0. To fit this model each possible segment with known break points corresponding to the V and J genes is transformed into vectors of 1s and 0s, which are equal to 1 within their region and 0 outside. These are fitted to the normalised read ratio data using a constrained linear model (using functions from the R package restriktor v0.3), with inequality constraints chosen as follows for each of the n V segments and m J segments:

$$\begin{matrix}{V}_{1} < 0 \\ {V}_{2} < {V}_{1} \\ … \\ {V}_{n} < {V}_{n - 1} \\ {J}_{1} > {V}_{n} \\ {J}_{2} > {J}_{1} \\ … \\ {J}_{m} > {J}_{m - 1} \\ {J}_{m} < 0\end{matrix}$$

Simultaneously to fitting this model for V(D)J recombination, values are also fitted to account for GC biases within the data. Using these fitted values the total B cell fraction can be calculated from the maximum deviation of the model e.g. the value from the last V segment as well as individual fractions from individual segments. To extend the model to the IGH locus for quantification of B cell fractions the model was modified to also include segments representing class switching deletion events (FIG. 7B) representing loss of gene segments resulting in different anti-body productions. The breakpoints of these deletion events were again restricted to match their genomic locations and there was an additional restriction that the total fraction of class switched B cells must be less or equal to that of the total B cell fraction as measured from the V(D)J region. The fraction for non-class switched igM/D b cells was calculated as the total B cell fraction minus the class switched B cell fraction.

Calculating of Shannon Diversity and Jensen-Shannon Divergence from B Cell Fractions Output

B cell diversity metrics from V or J segment usage or class-recombination as predicted by the model can be obtained as follows. For Shannon diversity we used the formula:

$$Shannon diversity = - {\sum }_{i} {p}_{i} ln ( {p}_{i} )$$

Where pᵢ represents the proportion of an individual V or J segment predicted from the model. To compare between two samples with different predicted segment usage we used the Jensen-Shannon divergence (JSD) metric defined as follows:

$$P = probability distribution of V or J segment usage in sample A$$

$$Q = probability distribution of V or J segment usage in sample B$$

$$M = \frac{P + Q}{2}$$

### D(P|Q)=Σᵢ

$${P}_{i} log ( \frac{{P}_{i}}{{Q}_{i}} )$$

where Pᵢ and Qᵢ represent the proportion of segment i used in sample A or B respectively.

The JSD is then defined as:

$$J S D ( P \vert Q ) = \frac{1}{2} D ( P \vert M ) + \frac{1}{2} D ( Q \vert M )$$

### 100KGP Date-Matched Blood Count Data

Blood count data was only available for the rare disease cohort within the 100KGP. We selected blood count data that was time matched for date of genomic sample collection resulting in data from 441 participants for which we had date-matched blood count and calculated T cell or B cell fractions. From this data we further subsetted for participants with matched albumin count (N=361), lymphocyte count (N=222), neutrophil count (N=84) and both neutrophil and lymphocyte count data (N=84).

### 100KGP Treatment Data

Treatment data was extracted from 100KGP using the clinical data available in the Genomics England research environment within from the ‘cancer_systemic_anti_cancer_therapy’ table.

### Survival Analysis

Survival data was collated on the GEL platform using available data for date of cancer diagnosis, death records from the office of national statistics (ONS) and latest follow up times from the most recent records in the hospital episode statistics (release v16). The data then underwent additional quality control to exclude any patients with any conflicting data such as follow-up times greater than 10 years resulting from multiple date of diagnosis values from previous incidences of cancer. In total, 15,179 participants with both survival data and B cell fractions in blood and 15,891 participants with both survival data and B cell fractions in tumour tissue were available for analysis.

Survival data was collated on the GEL platform using available data for date of cancer diagnosis, death records from the office of national statistics (ONS) and latest follow up times from the most recent records in the hospital episode statistics (release v16). The data then underwent additional quality control to exclude any patients with any conflicting data such as follow-up times greater than 10 years resulting from multiple date of diagnosis values from previous incidences of cancer. In total, 15,179 participants with both survival data and T cell ExTRECT fractions in blood and 15,891 participants with both survival data and T cell ExTRECT fractions in tumour tissue were available for analysis.

### 1000 Genome Cohort

2544 samples with matched high and low coverage cram files along with their indexed crai files were downloaded directly from the 1000 Genome cohort server (ftp://ftp.1000genomes.ebi.ac.uk) using wget. The present method with samtools was then used on these cram files to extract the coverage and then calculate T and B cell fractions. Processed RNAseq data from the Geuvadis project for 465 lymphoblastoid cell lines from the 1000 Genomes was downloaded from www.ebi.ac.uk/gxa/experiments/E-GEUV-1/Downloads with RNAseq analysis performed using R packages limma and edgeR.

### PCAWG

The analysis was restricted to the TCGA portion of the PCAWG that contained 533 WGS tumour normal pairs from bam files that had been realigned to hg38 at the MD Anderson. The present tool/Samtools was used to extract coverage files for the IGH locus which were then used in the calculation of all B cell fractions using the present tool's R package.

scRNA Cohort and Analysis

Processed scRNA data with associated metadata from a breast cancer dataset described in Wu et al. was downloaded from GSE176078. Annotation of B cell subtypes was used and class switching was determined from expression of IGH class switch segments with cells with unclear annotation removed from the analysis.

### 100KGP Ancestry Inference

Ancestry inference was done internally by Genomics England for the entire 100KGP cohort using ethnicities from the 1000 genome project phase 3 (1000GP3) as truth by first generating principal components and then projecting the 100KGP project onto them to identify the broad ancestry supercategory of each participant. Full details can be found at research-help.genomicsengland.co.uk/display/GERE/Ancestry+inference

### 100KGP Date-Matched Blood Count Data

Blood count data was only available for the rare disease cohort within the 100KGP. We selected blood count data that was time matched for date of genomic sample collection resulting in data from 441 participants for which we had date-matched blood count and calculated T cell or B cell fractions. From this data the inventors further subsetted for participants with matched albumin count (N=361), lymphocyte count (N=222), neutrophil count (N=84) and both neutrophil and lymphocyte count data (N=84).

### 100KGP Treatment Data

Treatment data was extracted from 100KGP using the clinical data available in the Genomics England research environment from the ‘cancer_systemic_anti_cancer_therapy’ version 13 table.

### REFERENCES

Bentham, R. et al. Using DNA sequencing data to quantify T cell fraction and therapy response. Nature 597, 555-560 (2021).
Danaher, P. et al. Pan-cancer adaptive immune resistance as defined by the Tumor Inflammation Signature (TIS): results from The Cancer Genome Atlas (TCGA). J Immunother Cancer 6, 63 (2018).
Davoli, T., Uno, H., Wooten, E. C. & Elledge, S. J. Tumor aneuploidy correlates with markers of immune evasion and with reduced response to immunotherapy. Science 355, (2017).
Jamal-Hanjani M, et al.; TRACERx Consortium. Tracking the Evolution of Non-Small-Cell Lung Cancer. N Engl J Med. 2017 Jun. 1; 376(22):2109-2121.
Hu, X. et al. Landscape of B cell immunity and related immune evasion in human cancers. Nat. Genet. 51, 560-567 (2019).
Law, C. W., Chen, Y., Shi, W. & Smyth, G. K. voom: Precision weights unlock linear model analysis tools for RNA-seq read counts. Genome Biol. 15, R29 (2014).
Li, T. et al. TIMER: A Web Server for Comprehensive Analysis of Tumor-Infiltrating Immune Cells. Cancer Res. 77, e108-e110 (2017).
Liberzon, A. et al. Molecular signatures database (MSigDB) 3.0. Bioinformatics vol. 27 1739-1740 Preprint at https://doi.org/10.1093/bioinformatics/btr260 (2011).
Newman, A. M. et al. Robust enumeration of cell subsets from tissue expression profiles. Nat. Methods 12, 453-457 (2015).
Oexle, K. Evaluation of the evenness score in next-generation sequencing. J Hum Genet 61, 627-632 (2016).
Renteria, M. E., Cortes, A., Medland, S. E. (2013). Using PLINK for Genome-Wide Association Studies (GWAS) and Data Analysis. In: Gondro, C., van der Werf, J., Hayes, B. (eds) Genome-Wide Association Studies and Genomic Prediction. Methods in Molecular Biology, vol 1019. Humana Press, Totowa, NJ.
Eric Roller, Sergii Ivakhno, Steve Lee, Thomas Royce, Stephen Tanner, Canvas: versatile and scalable detection of copy number variants, Bioinformatics, Volume 32, Issue 15, August 2016, Pages 2375-2377
Travaglini, K. J. et al. A molecular cell atlas of the human lung from single-cell RNA sequencing. Nature 587, 619-625 (2020).
Van Loo et al., 2010. Allele-specific copy number analysis of tumors. Proceedings of the National Academy of Sciences of the United States of America, 107(39), 1690-16915.
Song L, Cohen D, Ouyang Z, Cao Y, Hu X, Liu X S. TRUST4: immune repertoire reconstruction from bulk and single-cell RNA-seq data. Nat Methods. 2021 June; 18(6):627-630.
Bolotin D A, Poslavsky S, Mitrophanov I, Shugay M, Mamedov I Z, Putintseva E V, Chudakov D M. MiXCR: software for comprehensive adaptive immunity profiling. Nat Methods. 2015 May; 12(5):380-1.
Wu, S. Z. et al. A single-cell and spatially resolved atlas of human breast cancers. Nat. Genet. 53, 1334-1347 (2021).
Thorsson, V. et al. The Immune Landscape of Cancer. Immunity 51, 411-412 (2019).
Templeton, A. J. et al. Prognostic role of neutrophil-to-lymphocyte ratio in solid tumors: a systematic review and meta-analysis. J. Natl. Cancer Inst. 106, dju124 (2014).
Klein, S. L. & Flanagan, K. L. Sex differences in immune responses. Nat. Rev. Immunol. 16, 626-638 (2016).
Laskar, R. S. et al. Sexual dimorphism in cancer: insights from transcriptional signatures in kidney tissue and renal cell carcinoma. Hum. Mol. Genet. 30, 343-355 (2021).
Grecian, R., Whyte, M. K. B. & Walmsley, S. R. The role of neutrophils in cancer. British Medical Bulletin vol. 128 5-14 Preprint at https://doi.org/10.1093/bmb/Idy029 (2018).
Ronchetti, S., Ricci, E., Migliorati, G., Gentili, M. & Riccardi, C. How Glucocorticoids Affect the Neutrophil Life. Int. J. Mol. Sci. 19, (2018).
Nédélec, Y. et al. Genetic Ancestry and Natural Selection Drive Population Differences in Immune Responses to Pathogens. Cell 167, 657-669.e21 (2016).
Mikhaylova, A. V. et al. Whole-genome sequencing in diverse subjects identifies genetic correlates of leukocyte traits: The NHLBI TOPMed program. Am. J. Hum. Genet. 108, 1836-1851 (2021).
Chao, B. N., Carrick, D. M., Filipski, K. K. & Nelson, S. A. Overview of Research on Germline Genetic Variation in Immune Genes and Cancer Outcomes. Cancer Epidemiol. Biomarkers Prev. 31, 495-506 (2022).
1000 Genomes Project Consortium et al. A global reference for human genetic variation. Nature 526, 68-74 (2015).
Nosho, K. et al. Tumour-infiltrating T-cell subsets, molecular changes in colorectal cancer, and prognosis: cohort study and literature review. J. Pathol. 222, 350-366 (2010).
Hwang, W.-T., Adams, S. F., Tahirovic, E., Hagemann, I. S. & Coukos, G. Prognostic significance of tumor-infiltrating T cells in ovarian cancer: a meta-analysis. Gynecol. Oncol. 124, 192-198 (2012).
Kumarasamy, C. et al. Prognostic significance of blood inflammatory biomarkers NLR, PLR, and LMR in cancer-A protocol for systematic review and meta-analysis. Medicine vol. 98 e14834 Preprint at https://doi.org/10.1097/md.0000000000014834 (2019).
Carrot-Zhang, J. et al. Comprehensive Analysis of Genetic Ancestry and Its Molecular Correlates in Cancer. Cancer Cell 37, (2020).
Collins, A. M., Yaari, G., Shepherd, A. J., Lees, W. & Watson, C. T. Germline immunoglobulin genes: disease susceptibility genes hidden in plain sight? Curr Opin Syst Biol 24, 100-108 (2020).
Morbach, H., Eichhorn, E. M., Liese, J. G. & Girschick, H. J. Reference values for B cell subpopulations from infancy to adulthood. Clin. Exp. Immunol. 162, 271-279 (2010).
Byrska-Bishop, M. et al. High-coverage whole-genome sequencing of the expanded 1000 Genomes Project cohort including 602 trios. Cell 185, 3426-3440.e19 (2022).
Ryan, J. L. et al. Clonal evolution of lymphoblastoid cell lines. Lab. Invest. 86, 1193-1200 (2006).
Vettermann, C. & Schlissel, M. S. Allelic exclusion of immunoglobulin genes: models and mechanisms. Immunol. Rev. 237, 22 (2010).
Lappalainen, T. et al. Transcriptome and genome sequencing uncovers functional variation in humans. Nature 501, 506-511 (2013).

All references cited herein are incorporated herein by reference in their entirety and for all purposes to the same extent as if each individual publication or patent or patent application was specifically and individually indicated to be incorporated by reference in its entirety.

1. A computer-implemented method for determining the fraction of B lymphocytes of a particular class in a sample comprising genomic material from multiple cell types, the method comprising:
obtaining sequence data for the sample, the sequence data comprising a plurality of sequencing reads;
obtaining a read depth profile for the sample comprising read depths derived from the sequence data in a predetermined genomic region, wherein the predetermined genomic region includes at least a region of the IGH locus that undergoes class switch recombination and a region of the IGH locus that does not undergo class switch recombination;
obtaining a plurality of read depth ratios (rᵢ) by normalising the read depths in the predetermined genomic region by reference to a baseline read depth derived from a subset of the predetermined genomic region that does not undergo class switch recombination;
obtaining one or more summarised read depth ratio values (r_{B}) for respective portions of the region of the IGH locus that are likely to be deleted through class switch recombination; and
determining the fraction of B lymphocytes of a particular class in the sample as a function of the one or more summarised read depth ratio values (r_{B});
wherein the particular class of B lymphocytes is selected from: igG B cells, igA B cells, igE B cells, igM/D B cells and combinations thereof.

2. The method of claim 1, wherein the region of the IGH locus that undergoes class switch recombination comprises one or more segments selected from: IGHA2, IGHE, IGHG4, IGHG2, IGHA1, IGHG1, IGHG3, IGHD, and IGHM.

3. The method of claim 1 or claim 2, obtaining a summarised read depth ratio value (r_{B}) for a portion of the region of the IGH locus that is likely to be deleted through class switch recombination comprises obtaining a summarised read depth ratio value (r_{B}) for a portion of the IGH locus corresponding to a segment selected from: IGHA2, IGHE, IGHG4, IGHG2, IGHA1, IGHG1, IGHG3, IGHD, IGHM, and combinations thereof.

4. The method of any preceding claim, wherein the a baseline read depth is derived from a subset of the predetermined genomic region that is not expected to be lost in B cells through class switch recombination and/or VDJ recombination, optionally wherein the subsets of the predetermined region comprises one or both of: a region of the IGH locus that is located before segments that can be deleted through class switching, and a region of the IGH locus that is located after segments that can be deleted through VDJ recombination.

5. The method of claim 4, wherein a region of the IGH locus that is located before segments that can be deleted through class switching comprises a region before the IGHA2 segment of the IGH locus, and/or a region of the IGH locus that is located after segments that can be deleted through VDJ recombination comprises a region including the last n V segments of the IGH locus, optionally wherein the subsets of the predetermined region comprises one or both of: a region with coordinates Hg38: chr14:105566277-105588395 or corresponding coordinates in another human or non-human reference genome; and a region with coordinates Hg38: chr14: 106779812-106879844.

6. The method of any preceding claim, wherein obtaining a read depth profile for the sample comprises obtaining read depths along one or more segments from the constant region of the IGH locus, wherein the one or more segments have genomic coordinates selected from:
a) IGHA2, hg38 co-ordinates: chr14:105583731-105588395, or corresponding coordinates in another human or non-human reference genome;
b) IGHE, hg 38 coordinates: chr14:105597691-105601728, or corresponding coordinates in another human or non-human reference genome;
c) IGHG4, hg38 coordinates: chr14:105620506-105626066, or corresponding coordinates in another human or non-human reference genome;
d) IGHG2, hg38 coordinates: chr14:105639559-105644790, or corresponding coordinates in another human or non-human reference genome;
e) IGHA1, hg38 coordinates: chr14:105703995-105708665, or corresponding coordinates in another human or non-human reference genome;
f) IGHG1, hg38 coordinates: chr14:105736343-105743071, or corresponding coordinates in another human or non-human reference genome;
g) IGHG3, hg38 coordinates: chr14:105764503-105771405, or corresponding coordinates in another human or non-human reference genome;
h) IGHD, hg38 coordinates: chr14:105836765-105845677, or corresponding coordinates in another human or non-human reference genome; and
i) IGHM, hg38 coordinates: chr14:105851705-105856218, or corresponding coordinates in another human or non-human reference genome.

7. The method of any preceding claim, wherein the predetermined genomic region further includes a region of the IGH locus that undergoes VDJ recombination, and the method further comprises determining the total B lymphocyte fraction by:
obtaining a further summarised read depth ratio value (r_{B}) for a subset of the region of the IGH locus that is likely to be deleted through VDJ recombination; and
determining the total fraction of B lymphocytes (f^{tot}) in the sample as a function of the further summarised read depth ratio value (r_{B}).

8. The method of claim 7, wherein the subset of the region of the IGH locus that is likely to be deleted through VDJ recombination comprises a region located between the end of the last J segment of the genomic locus that undergoes VDJ recombination, and the start of the first V segment of the genomic locus that undergoes VDJ recombination, optionally wherein the subset of the IGH locus that is likely to be deleted through VDJ recombination comprises one or more D segments (such as e.g. all D segments) of the genomic locus that undergoes VDJ recombination and/or wherein the subset of the region of the IGH locus that is likely to be deleted through V(D)J recombination is Hg38: chr14: 105865679-105939755 or corresponding coordinates in another human or non-human reference genome.

9. The method of any preceding claim, wherein:
the portions of the region of the IGH locus that are likely to be deleted through class switch recombination are selected from portions of the IGH locus corresponding to a segment selected from: IGHA2, IGHE, IGHG4, IGHG2, IGHA1, IGHG1, IGHG3, IGHD, and IGHM; and
determining the fraction of B lymphocytes of a particular class in the sample as a function of the one or more summarised read depth ratio values (r_{B}) comprises determining, for each of one or more of said segments: the fraction of B lymphocytes (f^{class}) that include the segment as a function of the summarised read depth ratio value (r_{B}) associated with the segment.

10. The method of claim 9, wherein the summarised read depth ratio value (r_{B}) associated with a segment is indicative of the fraction of cells that include a deletion breakpoint before the segment, and wherein the particular class of B lymphocytes is:
the igG B cell class, the one or more summarised read depth ratio values (r_{B}) comprise summarised read depth ratio values (r_{B}) for respective portions of the region of the IGH locus each comprising a segment selected from IGHG1, IGHG2, IGHG3, and IGHG4, and the fraction of B lymphocytes in the igG B class is calculated as the sum of: the fraction of B lymphocytes that include a deletion breakpoint before the IGHG1 segment, the fraction of B lymphocytes that include a deletion breakpoint before the IGHG2 segment, the fraction of B lymphocytes that include a deletion breakpoint before the IGHG3 segment, and the fraction of B lymphocytes that include a deletion breakpoint before the IGHG4 segment; or
the igA B cell class, the one or more summarised read depth ratio values (r_{B}) comprise summarised read depth ratio values (r_{B}) for respective portions of the region of the IGH locus each comprising a segment selected from IGHA1 and IGHA2, and the fraction of B lymphocytes in the igA B class is calculated as the sum of: the fraction of B lymphocytes that include a deletion breakpoint before the IGHA1 segment and the fraction of B lymphocytes that include a deletion breakpoint before the IGHA2 segment; or
the igE B cell class, the one or more summarised read depth ratio values (r_{B}) comprise a summarised read depth ratio values (r_{B}) for a portion of the region of the IGH locus that comprises the IGHE segment, and the fraction of B lymphocytes in the IgE class is equal to the fraction of B lymphocytes that include a deletion breakpoint before the IGHE segment; or
the igM/D class, the one or more summarised read depth ratio values (r_{B}) comprise summarised read depth ratio values (r_{B}) for respective portions of the region of the IGH locus each comprising a segment selected from IGHG1, IGHG2, IGHG3, IGHG4, IGHA1, IGHA2 and IGHE, the method comprises determining the total B lymphocyte fraction by obtaining a further summarised read depth ratio value (r_{B}) for a subset of the region of the IGH locus that is likely to be deleted through VDJ recombination, and determining the total fraction of B lymphocytes (f^{tot}) in the sample as a function of the further summarised read depth ratio value (r_{VDJ}), and the fraction of B lymphocytes in the igM/D class is calculated as the total B lymphocyte fraction minus the sum of: the fraction of B lymphocytes that include a deletion breakpoint before the IGHG1 segment, the fraction of B lymphocytes that include a deletion breakpoint before the IGHG2 segment, the fraction of B lymphocytes that include a deletion breakpoint before the IGHG3 segment, the fraction of B lymphocytes that include a deletion breakpoint before the IGHG4 segment, the fraction of B lymphocytes that include a deletion breakpoint before the IGHA1 segment, the fraction of B lymphocytes that include a deletion breakpoint before the IGHA2 segment, and the fraction of B lymphocytes that include a deletion breakpoint before the IGHE segment.

11. The method of any preceding claim, wherein the method comprises determining the fraction of B lymphocytes of a plurality of classes, wherein the classes comprise: igG B cells, igA B cells, igE B cells and igM/D B cells, optionally wherein the method comprises determining the fraction of class switched B lymphocytes as the sum of the igG, igA, and igE B lymphocyte fractions.

12. The method of any preceding claim, wherein:
determining the fraction of B lymphocytes of a particular class in the sample as a function of the one or more summarised read depth ratio values (r_{B}) comprises determining, for each of one or more segments of the IGH gene for which a summarised read depth ratio values (r_{B}) has been obtained, the fraction of B lymphocytes (f^{class}) that include a deletion breakpoint before the segment as a function of the summarised read depth ratio value (r_{B}) associated with the segment, and/or wherein the method comprises determining the total fraction of B lymphocytes by obtaining a further summarised read depth ratio value (r_{B}) for a subset of the region of the IGH locus that is likely to be deleted through VDJ recombination and determining the total fraction of B lymphocytes (f^{tot}) in the sample as a function of the further summarised read depth ratio value (r_{B}).

13. The method of claim 12, wherein the fraction of B lymphocytes (f^{class}) that include a deletion breakpoint before the segment and/or the total fraction of B lymphocytes (f^{tot}) and/or the total fraction of class switched B lymphocytes (F^{class}) is determined as a function of the respective summarised read depth ratio value, the fraction of abnormal cells (p) in the mixed sample, where abnormal cells are those that are aneuploid in the region of the IGH locus, and the copy number of the abnormal cells in the region of the IGH locus (ψ^{T}).

14. The method of claim 12 or claim 13, wherein determining the fraction of B lymphocytes that include a deletion breakpoint before the segment (f=f^{class}) in the sample and/or determining the total fraction of B lymphocytes (f=f^{tot}) and/or the total fraction of class switched B lymphocytes (F^{class}) comprises determining the value of:
$$f = ( 1 - p + \frac{p}{2} {Ψ}_{T} ) - ( 1 - p + \frac{p}{2} {Ψ}_{T} ) {2}^{\frac{{r}_{B}}{γ}} \qquad ( 7 )$$
where γ is a constant; or
$$f = 1 - {2}^{\frac{{r}_{B}}{γ}} \qquad ( 3 )$$
where γ is a constant.

15. The method of any preceding claim, further comprising fitting a model to the plurality of read depth ratios (rᵢ), and wherein obtaining one or more summarised read depth ratio values for respective portions of the region of the IGH locus that are likely to be deleted through class switch recombination comprises obtaining the one or more summarised read depth ratio values based on the value of the model in the respective portion of the region of the IGH locus that is likely to be deleted through class switch recombination, optionally wherein the model is a general linear model, a piecewise constant linear model with a plurality of breakpoints selected to correspond to locations of a plurality of segments in the constant region of the IGH locus, or a Bayesian model modelling segment usage from the read depth ratios and a prior distribution of usage of each of a plurality of segments in the constant region of the IGH locus.

16. The method of claim 15, wherein the model is a piecewise constant linear model fitted to the plurality of read depth ratios using one or more of the following constraints:
breakpoints between constant sections are restricted to locations of the ends of a plurality of segments in the constant region of the IGH locus,
the read depth ratio value at the end of the IGHM segment in the IGH locus must be 0, and
the read depth ratio after the IGHG3 segment in the IGH locus must be less or equal to the read depth ratio for a subset of the region of the IGH locus that is likely to be deleted through VDJ recombination.

17. The method of any one of claims 7-16, wherein the method comprises fitting a model to the plurality of read depth ratios (rᵢ), and wherein obtaining a further summarised read depth ratio (r_{B}) for a subset of the region of the IGH locus that is likely to be deleted through VDJ recombination comprises obtaining the further summarised read depth ratio value based on the value of the model in the subset of the region of the IGH locus that is likely to be deleted through VDJ recombination, optionally wherein the model is a general linear model, a piecewise constant linear model with a plurality of breakpoints selected to correspond to locations of a plurality of V and J genes in the IGH locus, or a Bayesian model modelling segment usage from the read depth and a prior distribution of usage of each of a plurality of V and J genes in the IGH locus.

18. The method of claim 17, wherein the model is a piecewise constant linear model fitted to the plurality of read depth ratios using one or more of the following constraints: breakpoints between constant sections are restricted to locations of the ends of a plurality of V and J segments in the IGH locus, the read depth ratio value before the first V segment must be 0, the read depth ratio value must decrease monotonically between the first V segment and the last V segment, the read depth ratio value must increase monotonically between the first J segment and the last J segment, and the read depth ratio value after the last J segment must be 0.

19. The method of any preceding claim, wherein a summarised read depth ratio value (r_{B}) for a portion of the region of the IGH locus that is likely to be deleted through class switch recombination is obtained as the difference or absolute value of a difference between a read depth ratio value associated with the portion of the region of the IGH locus and a read depth ratio value associated with the preceding portion of the region of the IGH locus.

20. The method of any preceding claim, wherein the sequence data is from whole genome sequencing, optionally wherein the sequence data is from whole genome sequencing with a depth of at least 2×, preferably at least 5×.

21. The method of any preceding claim, obtaining a read depth profile for the sample comprises obtaining raw read depth values from the sequence data in the predetermined genomic region and correcting said raw read depth values for GC content and/or for copy number alterations in the predetermined region.

22. The method of claim 21, wherein the sample is a germline sample and obtaining a read depth profile for the sample comprises obtaining read depth values from the sequence data in the predetermined genomic region corrected for germline copy number alterations in the predetermined region by:
dividing the raw read depth values in the predetermined region, optionally the IGH locus, by the median read depth in the predetermined region,
smoothing the resulting read depth values using a rolling average over windows of a predetermined size along the predetermined genomic region,
rounding the resulting read depth values to the nearest 0.5 value over windows of a predetermined size along the predetermined genomic region, and
setting the raw read depths to zero in regions where the rounded read depth values indicate a loss of the region on both alleles, and dividing the raw read depth values by 0.5 in regions where the rounded read depth values indicate the loss of the region in one allele, by 1.5 in regions where the rounded read depth values indicate the gain of the region in one allele, and by 2 in regions where the rounded read depth values indicate a duplication of the region.

23. The method of claim 21, wherein the sample is a tumour sample and obtaining a read depth profile for the sample comprises obtaining read depth values from the sequence data in the predetermined genomic region corrected for somatic copy number alterations in the predetermined region by:
identifying one or more regions with a somatic copy number alteration in the IGH locus using the read depth profile for the sample and a read depth profile from a matched germline sample, and
dividing the read depths within the one or more regions identified as having a somatic copy number alteration by the ratio of the median read depth value within the region to the median read depth value across the IGH locus,
optionally wherein identifying somatic copy number alterations in the IGH locus using the read depth profile for the sample and a read depth profile from a matched germline sample comprises:
obtaining a GC corrected read depth profile for the sample and a GC corrected read depth profile from the matched germline sample,
normalising each of said read depth profiles by dividing read depth values by the respective median value across the IGH locus,
obtaining a log R profile as the log 2 ratio of the normalised read depth profiles for the tumour and germline samples, and
identifying segments in said log R profile, optionally by obtaining median values for each of a plurality of windows of predetermined size and performing recursive partitioning to identify segments, wherein a region identified as having a somatic copy number alteration is a region associated with a segment of a length above a predetermined threshold that has a log R different from 1.

24. The method of claim 23, wherein obtaining a read depth profile for the sample comprises:
obtaining read depth values from the sequence data in the predetermined genomic region corrected for somatic copy number alterations in the predetermined region, and
obtaining read depth values from the sequence data in the predetermined genomic region further corrected for germline copy number alterations in the predetermined region by:
identifying regions associated with a germline copy number alteration using a read depth profile from a matched germline sample; and
dividing the read depths values corrected for somatic copy number alterations on regions associated with a germline copy number alteration by the ratio of the median read depth within the region in the tumour sample and the median read depth across the IGH locus in the tumour sample.

25. The method of any preceding claim, wherein the sample is a sample from a subject who has or is suspected of having an autoimmune disease, optionally wherein the sample is a blood sample or a tissue sample, or wherein the sample is a sample from a subject who has been diagnosed as having cancer, optionally wherein the sample is a blood sample or a tumour sample, and/or wherein the method further comprises obtaining a sample comprising genomic material from multiple cell types, from a subject, and/or obtaining sequence data from a sample comprising genomic material from multiple cell types, from a subject through one or more in vitro steps.

26. The method of any preceding claim, further comprising providing to a user, for example through a user interface, the determined one or more B lymphocyte fractions and/or any value derived therefrom.

27. A method of providing a prognosis for a subject that has been diagnosed as having cancer, the method comprising determining the fraction of B lymphocytes of a particular class in one or more tumour or blood samples from the subject using the method of any of claims 1 to 26.

28. The method of claim 27, wherein the method comprises determining the fraction of igM/D B lymphocytes in a blood sample from the subject, and determining whether the subject belongs to a first group associated with a first range of values of the fraction of igM/D B lymphocytes or a second group associated with a second, lower range of values of the fraction of igM/D B lymphocytes, wherein the first group is associated with a better prognosis than the second group.

29. A method of diagnosing a subject as having an auto-immune disease characterised by an increase in the fractions of B lymphocytes of one or more particular classes, the method comprising determining the fraction of B lymphocytes of the one or more particular classes in a sample from the subject using the method of any one of claims 1 to 26.

30. A system comprising:
a processor; and
a computer readable medium comprising instructions that, when executed by the processor, cause the processor to perform the steps of the method of any of claims 1 to 29.
